(19) United States
(12) Patent Application Publication
(10) Pub. No.: US 2026/0062495 A1
(43) Pub. Date: Mar. 5, 2026

(54) COMMERCIAL-SCALE RECOMBINANT PROTEIN PRODUCTION IN RAT HYBRIDOMA CELLS

(71) Applicants: TG THERAPEUTICS, INC., New York, NY (US); LABORATOIRE FRANCAIS DU FRACTIONNEMENT ET DES BIOTECHNOLOGIES, Les Ulis (FR)

(72) Inventors: George Costas AVGERINOS, Sudbury, MA (US); Patrick Michael HOSSLER, Westborough, MA (US)

(21) Appl. No.: 19/099,752

(22) PCT Filed: May 31, 2023
(86) PCT No.: PCT/US2023/067707

§ 371 Date: Jan. 29, 2025

Related U.S. Application Data
(60) Provisional application No. 63/347,793, filed on Jun. 1, 2022.

(51) Int. Cl. C07K 16/28; C12P 21/02

(52) U.S. Cl. CPC C07K 16/2887; C12P 21/02; C07K 2317/41; C07K 2317/732; C07K 2317/734; C07K 2317/92

(57) ABSTRACT

The present disclosure relates to methods of making recombinant proteins (e.g., monoclonal antibodies) in rat hybridoma cells (e.g., YB2/0) with high productivity, product quality, and robust functional activity by modifying cell culture process parameters and/or basal media and feed combinations. The present disclosure also relates to commercial-scale production (e.g., 10,000 L-25,000 L) of recombinant proteins (e.g., monoclonal antibodies) in rat hybridoma cells. The present disclosure also relates to recombinant proteins (e.g., monoclonal antibodies) and compositions (e.g., pharmaceutical) that are made using the methods disclosed herein, and which have a unique glycosylation profile.

### CLAIM OF PRIORITY

This application claims the benefit of U.S. Provisional Application Ser. No. 63/347,793, filed on Jun. 1, 2022. The entire contents of the foregoing are hereby incorporated by reference.

### REFERENCE TO SEQUENCE LISTING SUBMITTED ELECTRONICALLY

This application contains a Sequence Listing that has been submitted electronically as an XML file named “50581-0003WO1.XML.” The XML file, created on May 22, 2023, is 19,157 bytes in size. The material in the XML file is hereby incorporated by reference in its entirety.

### FIELD OF THE DISCLOSURE

The present disclosure is in the field of mammalian cell culture for producing recombinant proteins (e.g., monoclonal antibodies). More specifically, the present disclosure is in the field of commercial-scale production (e.g., 10,000 L-25,000 L) of recombinant proteins (e.g., monoclonal antibodies) in rat hybridoma cells.

### BACKGROUND OF THE DISCLOSURE

Recombinant proteins (e.g., antibodies) have become increasingly important as therapeutic agents in a wide range of diseases, such as cancer, autoimmune, and infectious diseases. See, Kaplon, H. et al., MAbs 11:219-238 (2019); Lu, R-M. et al., Journal of Biomedical Science 27:1 (2020). Around the world, at least 570 therapeutic mAbs have been studied in clinical trials, and as of December 2019, 79 therapeutic mAbs have been approved by the U.S. Food and Drug Administration (FDA) and are currently on the market, including 30 mAbs for the treatment of cancer. Id.

In most cases, therapeutic proteins (e.g., antibodies) are produced in cell culture, from mammalian cells that have been engineered and/or selected to produce high levels of the polypeptide of interest. Indeed, among all approved recombinant protein-based biopharmaceuticals, mammalian cells dominate other recombinant protein-expression systems (Owczarek, B. et al., BioMed Research International 2019: Article ID 4216060, 1-13 (2019). Chinese hamster ovary (CHO) cells, murine hybridoma cells (NS0), and mouse hybridoma (Sp2/0) cells are the main mammalian cell lines used to express recombinant biopharmaceuticals, with CHO-based systems contributing the largest percentage (about 84%) (Tripathi, N. K. et al., Front Bioeng Biotechnol. 7: 420 (2019)).

Rat hybridoma cell lines have also been used, albeit much less frequently than other cell lines for producing therapeutic antibodies. For example, a rat hybridoma cell line, YB2/0 has been used for the production of anti-Rh(D) monoclonal antibodies with enhanced antibody-dependent cellular cytotoxicity (ADCC) function, which can be used to prevent Rhesus isoimmunization in Rh negative individuals. See, U.S. Pat. Nos. 7,931,895 and 8,409,572, assigned to LFB Biotechnologies. U.S. Pat. No. 9,234,045, assigned to Laboratoire Francais du Fractionnement et des Biotechnologies, is directed to a monoclonal antibody directed against the CD20 antigen, which is produced by a rat hybridoma YB2/0 cell line, amongst other cell lines. These patents are incorporated by reference herein in their entireties. There is still a need, however, to produce monoclonal antibodies in rat hybridoma cells at a commercial-scale.

In mammalian cell culture, media formulations, cell lines, cell culture, and process control parameters can potentially impact the resulting recombinant protein yields and product quality profiles. See, Li, F. et al., Mabs 2:466-477 (2010). Characterization and understanding of culture process parameters, e.g., culture pH, dissolved CO₂ (pCO₂), temperature, culture media, on process performance and product quality attributes is a principal reason why these relationships are interrogated during process characterization studies, and tracked during good manufacturing process (GMP) manufacturing. Also, a relationship that exists with one cell line does not necessarily hold true for another. Jiang, R. et al., Bioprocess Biosyst Eng 41:1731-1741 (2018). Process parameter acceptance limits are defined by manufacturers of therapeutic proteins to ensure that products with the desired specifications are achieved consistently.

Protein glycosylation is a post-translational modification (PTM) that can impact the product quality of recombinant proteins. Asparagine-linked (N-linked) glycosylation is very common on recombinant therapeutic glycoproteins, especially antibodies. N-linked protein glycosylation is typically comprised of 5 principal sugars (FIG. 1) and has been shown to have a determining role towards the physiochemical, pharmacokinetic, immunogenic, Fc effector function of the proteins to which they are attached. Accordingly, protein glycosylation (e.g., fucosylation) is usually categorized as a critical quality attribute (CQA) during bioprocessing and is closely monitored during manufacturing to ensure adherence to sponsor-defined acceptance limits.

Optimizing mammalian cell culture process parameters is important for successful, cost-effective, and reproducible commercial production of recombinant proteins with desired specifications, increased titer, high product quality profiles (e.g., low fucosylation), and robust biological activity. Accordingly, there is a need for improving both recombinant protein productivity and product quality in mammalian cell expression systems, especially for therapeutic monoclonal antibodies produced on a commercial-scale.

### SUMMARY OF THE DISCLOSURE

Provided herein are methods for making recombinant proteins (e.g., monoclonal antibodies) in an uncommonly-used mammalian expression system—the rat hybridoma cell line, e.g., YB2/0. Also provided are methods for increasing product titer and improving product quality and functional activity of those recombinant proteins by modifying cell culture process parameters and/or cell culture media. Also provided are methods for commercial-scale production (e.g., 10,000 L-25,000 L) of recombinant proteins (e.g., monoclonal antibodies) in rat hybridoma cells that retain high protein quality and functional activity. In a particular aspect, the methods of the present disclosure are used to produce anti-CD20 antibodies on a commercial-scale. Also provided herein are compositions made in accordance with the presently disclosed methods, including pharmaceutical compositions. In some aspects, the compositions made in accordance with the presently disclosed methods exhibit uniqe glycosylation patterns, which contribute to consistent product quality, clinical safety, and efficacy.

Accordingly, provided herein is a method of producing at least 10,000 L of an antibody protein in rat hybridoma cells by culturing the rat hybridoma cells in a cell culture having a culture pH of about 6.5 to about 7.55, wherein the rat hybridoma cells comprise an expression vector comprising a polynucleotide encoding the antibody protein.

In some embodiments, the culture pH is about 6.5 to about 7.0. In some embodiments, the culture pH of about 6.5 to about 7.0 is set on culture day 2 of the cell culture. In some embodiments, the culture pH of about 6.5 to about 7.0 is set on culture day 3 of the cell culture.

In some embodiments, the culture pH is about 7.0 to about 7.55. In some embodiments, the culture pH of about 7.0 to about 7.55 is set on culture days 0 to 3 of the cell culture.

In some embodiments, the culture pH is lowered on day 2 or day 3 of the cell culture to about 6.5 to about 7.0. In some embodiments, the culture pH is lowered on day 3 of the cell culture. In some embodiments, the culture pH of about 6.5 to about 7.0 is maintained from culture day 3 until harvest of the cell culture.

In some embodiments, the cumulative culture time that pH is allowed to drop below a fixed pH setpoint and the cumulative magnitude of the drop, after the pH is lowered on day 3 (integrated pH2 difference), is less, relative to a cell culture with an integrated pH2 difference that is more. In some embodiments, the fixed pH setpoint is pH 6.91.

In some embodiments, lower integrated pH2 difference results in higher integrated viable cell density (IVCD) and higher titer at harvest. In some embodiments, lower integrated pH2 difference further results in lower percent fucosylation.

In some embodiments, the rat hybridoma cells expressing the antibody protein are cultured in a chemically defined and animal-derived component free (ADCF) culture medium.

In some embodiments, harvest titer of the antibody protein is increased and/or fucosylation of the antibody protein is decreased when the culture pH is 6.6 to 6.96 relative to a cell culture under the same culture conditions except that the culture pH is 6.60 to 6.8.

In some embodiments, the method further comprises controlling culture pCO₂ levels to less than about 300 mmHg. In some embodiments, pCO₂ levels less than about 300 mmHg are facilitated by supplementing the cell culture with additional buffers, increasing the air sparge rate, increasing the dissolved oxygen (DO) setpoint, and/or decreasing the agitation rate.

In some embodiments, the method further comprises an initial temperature set point of about 37° C., wherein said initial temperature set point is set on culture day 0 to culture day 1. In some embodiments, the method further comprises a second temperature set point of about 35° C., wherein said second temperature set point is set at the end of culture day 1 to culture day 3.

In some embodiments, the end of culture day one is 17 to 33 hours after the start of the cell culture.

In some embodiments, the method further comprises a third temperature set point of about 32° C. to about 33° C., wherein said third temperature set point is set on culture day 3 and is maintained though harvest. In some embodiments, the third temperature set point is 32.5° C.

In some embodiments, the cell culture comprises the following culture conditions: i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set on culture day 3 and is maintained though harvest; ii) a culture pH between about 6.5 to about 7.55; and iii) a culture pCO₂ less than about 300 mmHg.

In some embodiments, yield of the antibody protein is increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to an antibody protein produced by a culturing process that does not employ the culture conditions recited here.

In some embodiments, the method further comprises harvesting the antibody protein produced by the rat hybridoma cell.

In some embodiments, the method further comprises purifying the antibody protein by affinity chromatography and/or ion exchange chromatography. In some embodiments, the affinity chromatography comprises protein A purification. In some embodiments, the purified antibody protein produced by the rat hybridoma cell is formulated into a pharmaceutically acceptable formulation. In some embodiments, quality of the purified antibody protein is measured by SEC-HPLC, imaged capillary electrophoresis (ICIEF), and/or N-linked glycan analysis.

In some embodiments, the antibody protein is a monoclonal antibody. In some embodiments, the antibody protein (e.g., the monoclonal antibody) is an anti-CD20 antibody.

In some embodiments, the monoclonal antibody is subject to CD20, FcγRIIIa-158V, and/or C1q binding assays.

In some embodiments, the monoclonal antibody has a relative potency of 82% to 138% in a cell-based CD20 binding activity bioassay compared to that of a commercial reference standard. In some embodiments, CD20 binding is determined by the binding of an anti-CD20 antibody to the CD20 expressing human mantle cell lymphoma cell line, Jeko-1.

In some embodiments, the percentage of FcγRIIIa-158V binding is about 82% to about 130% with respect to a commercial reference standard for the binding assay. In some embodiments, the percentage of FcγRIIIa-158V binding is determined by surface plasmon resonance (SPR).

In some embodiments, the monoclonal antibody has a relative potency of 86% to 117% in a C1q binding assay as measured by ELISA compared to a commercial reference standard. In some embodiments, the monoclonal antibody has a relative potency of 88% to 113% in a C1q binding assay as measured by ELISA compared to a commercial reference standard.

In some embodiments, the monoclonal antibody has a relative potency of 74% to 127% in a cell-based complement dependent cytotoxicity (CDC) assay compared to that of a commercial reference standard.

In some embodiments, the monoclonal antibody produced has higher percent antibody-dependent cellular cytotoxicity (ADCC) activity relative to a monoclonal antibody produced by a culturing process that does not employ the culture conditions recited here. In some embodiments, the monoclonal antibody has a relative potency of 90% to 163% in a cell-based ADCC assay compared to a commercial reference standard. In some embodiments, the monoclonal antibody has a relative potency of about 117% in a cell-based ADCC assay compared to a commercial reference standard.

In some embodiments, the monoclonal antibody comprises: a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some embodiments, the monoclonal antibody comprises a heavy chain having at least 95% identity to an amino acid sequence set forth in SEQ ID NO:7 and a light chain having at least 95% identity to an amino acid sequence set forth in SEQ ID NO:8. In some embodiments, the monoclonal antibody comprises a heavy chain having an amino acid sequence set forth in SEQ ID NO:7 and a light chain having an amino acid sequence set forth in SEQ ID NO:8. In some embodiments, the monoclonal antibody comprises a heavy chain having an amino acid sequence set forth in SEQ ID NO:7 and a light chain having an amino acid sequence set forth in SEQ ID NO:9.

In some embodiments, the monoclonal antibody comprises a deletion of up to 5 N-terminal residues. In some embodiments, the monoclonal antibody comprises a deletion of up to 10 N-terminal sequences.

In some embodiments, the cell culture is conducted in a bioreactor. In some embodiments, the bioreactor is a commercial-scale bioreactor. In some embodiments, the commercial-scale bioreactor is a 10,000 L, 15,000 L, 20,000 L, or 25,000 L bioreactor. In some embodiments, the commercial-scale bioreactor is a 15,000 L bioreactor.

In some embodiments, the rat hybridoma cell is a YB2/0 rat hybridoma cell.

Also provided here is a method of making an antibody protein in a culture of rat hybridoma cells at a commercial-scale comprising the steps of: a) preparing and thawing a working rat hybridoma cell bank of the antibody protein of interest; b) expanding a culture of the rat hybridoma cells from the cell bank by size and volume though a series of shake flasks (125 mL, 500 mL, 3 L, 3×3 L shake flasks, and 50 L cellbag) with a targeted seeding density of at least 0.30×10⁶ viable cells/mL; c) processing the cell culture through a series of seed bioreactors (120 L, 600 L, and 3,000 L) to further increase the volume and cell culture mass; d) inoculating the cell culture from the 3,000 L seed bioreactor into a commercial-scale production bioreactor; e) harvesting the cell culture supernatant from the commercial scale production bioreactor; f) clarifying the recovered cells by continuous centrifugation followed by depth filtration; g) purifying the antibody protein by Protein A capture column chromatography; and h) inactivating viral agents by solvent detergent viral inactivation (SDVI).

In some embodiments, the commercial-scale production bioreactor is operated in fed-batch mode.

In some embodiments, a drilled hole (10) gas sparger with 4.0 mm orifice diameter is used in the commercial-scale production bioreactor.

In some embodiments, the method further comprises purification by cation exchange chromatography (CEX) and anion exchange chromatography (AEX).

In some embodiments, the method further comprises viral filtration (VF) to remove potential viruses.

In some embodiments, the method further comprises ultrafiltration/diafiltration (UFDF).

In some embodiments, the method further comprises preparing a bulk drug substance formulation comprising the antibody protein by adding polysorbate 80 in formulation buffer to prepare a bulk drug substance formulation. In some embodiments, the method further comprises subjecting the bulk drug substance formulation to 0.2 μm filtration. In some embodiments, the method further comprises filling a 6 L bag to a target fill volume of 5.50 L of the bulk drug substance formulation and storing the bulk drug substance formulation at ≤−35° C. In some embodiments, the antibody protein in the bulk drug substance formulation is formulated into a pharmaceutically acceptable formulation. In some embodiments, the method further comprises testing unprocessed bulk harvest from the commercial-scale production bioreactor for microbial and viral adventitious agents. In some embodiments, the method further comprises removing and/or inactivating the microbial and viral adventitious agents from the commercial-scale production bioreactor.

In some embodiments, the rat hybridoma cells are YB2/0 cells.

In some embodiments, the antibody protein is a monoclonal antibody. In some embodiments, the antibody protein (e.g., the monoclonal antibody) is an anti-CD20 antibody.

In some embodiments, the monoclonal antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some embodiments, the monoclonal antibody comprises a heavy chain having at least 95% identity to an amino acid sequence set forth in SEQ ID NO:7; and a light chain having at least 95% identity to an amino acid sequence set forth in SEQ ID NO: 8.

In some embodiments, the monoclonal antibody comprises a heavy chain having an amino acid sequence set forth in SEQ ID NO:7; and a light chain having an amino acid sequence set forth in SEQ ID NO:8. In some embodiments, the monoclonal antibody comprises a heavy chain having an amino acid sequence set forth in SEQ ID NO:7; and a light chain having an amino acid sequence set forth in SEQ ID NO:9.

In some embodiments, the antibody protein comprises an N-glycan profile comprising one or both of the following: i) about 10 to 20% galactosylated glycans; and/or ii) about 20 to 40% fucosylated glycans.

In some embodiments, the N-glycan profile comprises about 10 to 20% galactosylated glycans and about 23% to 36% fucosylated glycans. In some embodiments, the N-glycan profile comprises about 23% to about 36% fucosylated glycans. In some embodiments, the N-glycan profile comprises about 16% to about 18% galactosylated glycans. In some embodiments, the N-glycan profile comprises about 17% galactosylated glycans.

In some embodiments, the antibody protein comprises an N-glycan profile comprising at least about 10% bisecting N-glycans. In some embodiments, the N-glycan profile comprises about 12% to about 30% bisecting N-glycans. In some embodiments, the N-glycan profile comprises about 18% bisecting N-glycans.

In some embodiments, the antibody protein comprises an N-glycan profile comprising less than 5% sialylated glycans. In some embodiments, the N-glycan profile comprises less than 4%, 3%, 2.5%, 2%, 1%, or 0.5% sialylated glycans. In some embodiments, the N-glycan profile comprises no detectable amount of sialylated glycan.

In some embodiments, the antibody protein comprises an N-glycan profile comprising 0.1% to 1.5% Man5 N-glycan. In some embodiments, the N-glycan profile comprises 0.4% to 0.7% Man5 N-glycan. In some embodiments, the N-glycan profile comprises about 0.6% Man5 N-glycan. In some embodiments, Man5 N-glycan is the only high mannose species in the N-glycan profile.

In some embodiments, the antibody protein is produced at a commercial-scale of about 10,000 L-about 25,000 L. In some embodiments, the commercial-scale is 15,000 L.

In some embodiments, the method yields an antibody protein harvest titer of about 0.5 g/L to about 1.5 g/L. In some embodiments, the harvest titer is about 1.0 g/L to about 1.5 g/L.

Also provided here is an antibody protein made according to the method described herein. In some embodiments, the antibody protein is a monoclonal antibody. In some embodiments, the antibody protein (e.g., the monoclonal antibody) is an anti-CD20 antibody.

Also provided here is a rat hybridoma master cell bank (MCB) composition comprising an antibody protein having at least two of the following parameters: i) peak viable cell density of about 11 to about 13×10⁶ cells/mL; ii) harvest titer of about 650 to about 720 mg/L; iii) percent fucosylation of about 30% to about 38%; iv) about 97% to about 99% monomers as detected by size exclusion chromatography (SEC); v) about 1.5% to about 2% dimers as detected by SEC; vi) undetectable to about 3% level of aggregates as detected by SEC; vii) undetectable to about 1% level of fragments as detected by SEC; viii) about 25% to about 30% acidic isoforms as detected by imaged capillary isoelectric focusing (iCIEF); ix) about 38% to about 49% main isoforms as detected by iCIEF; and/or x) about 20% to about 36% basic isoforms as detected by iCIEF.

Also provided here is a rat hybridoma working cell bank (WCB) composition comprising an antibody protein having at least two of the following parameters: i) peak viable cell density that is about 11 to about 28×10⁶ cells/mL; ii) harvest titer that is about 420 to about 1280 mg/L; iii) percent fucosylation of about 18% to about 40%; iv) about 97% to about 99% monomers as detected by size exclusion chromatography (SEC); v) about 1% to about 2% dimers as detected by SEC; vi) undetectable to about 2% level of aggregates as detected by SEC; vii) undetectable to about 1% level of fragments as detected by SEC; viii) about 19% to about 31% acidic isoforms as detected by imaged capillary isoelectric focusing (iCIEF); ix) about 34% to about 62% main isoforms as detected by iCIEF; and/or x) about 14% to about 38% basic isoforms as detected by iCIEF.

In some embodiments, the rat hybridoma cells in the cell bank are YB2/0 cells. In some embodiments, the antibody protein is a monoclonal antibody. In some embodiments, the antibody protein (e.g., the monoclonal antibody) is an anti-CD20 antibody.

In some embodiments, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some embodiments, the anti-CD20 antibody comprises a heavy chain having at least 95% identity to an amino acid sequence set forth in SEQ ID NO:7; and a light chain having at least 95% identity to an amino acid sequence set forth in SEQ ID NO: 8. In some embodiments, the anti-CD20 antibody comprises a heavy chain having an amino acid sequence set forth in SEQ ID NO:7; and a light chain having an amino acid sequence set forth in SEQ ID NO:8. In some embodiments, the anti-CD20 antibody comprises a heavy chain having an amino acid sequence set forth in SEQ ID NO:7; and a light chain having an amino acid sequence set forth in SEQ ID NO:9.

Also provided here are methods of making an antibody protein by using the MCB or WCB composition described here.

In some embodiments, the antibody protein is a monoclonal antibody. In some embodiments, the antibody protein (e.g., the monoclonal antibody) is an anti-CD20 antibody.

In some embodiments, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some embodiments, the anti-CD20 antibody comprises a heavy chain having at least 95% identity to an amino acid sequence set forth in SEQ ID NO:7; and a light chain having at least 95% identity to an amino acid sequence set forth in SEQ ID NO: 8. In some embodiments, the anti-CD20 antibody comprises a heavy chain having an amino acid sequence set forth in SEQ ID NO:7; and a light chain having an amino acid sequence set forth in SEQ ID NO:8. In some embodiments, the anti-CD20 antibody comprises a heavy chain having an amino acid sequence set forth in SEQ ID NO:7; and a light chain having an amino acid sequence set forth in SEQ ID NO:9.

In some embodiments, the method of making a recombinant protein in rat hybridoma cells comprises culturing the rat hybridoma cells in a cell culture having a culture pH of about 6.5 to about 7.55, wherein the rat hybridoma cells comprise an expression vector comprising a polynucleotide encoding the recombinant protein. In some aspects, the culture pH is about 6.5 to about 7.0. In some aspects, the culture pH of about 6.5 to about 7.0 is set on culture day 2 of the cell culture. In some aspects, the culture pH of about 6.5 to about 7.0 is set on culture day 3 of the cell culture. In some aspects, the culture pH is about 7.0 to about 7.55. In some aspects, the culture pH of about 7.0 to about 7.55 is set on culture days 0 to 3 of the cell culture.

In some embodiments, the culture pH is lowered on day 2 or day 3 of the cell culture to about 6.5 to about 7.0. In some embodiments, the culture pH is lowered on day 3 of the cell culture. In some embodiments, the culture pH of about 6.5 to about 7.0 is maintained from culture day 3 until harvest of the cell culture.

In some embodiments, the cumulative culture time that pH is allowed to drop below a fixed pH setpoint and the cumulative magnitude of the drop, after the pH is lowered on day 3 (integrated pH2 difference), is less, relative to a cell culture with an integrated pH2 difference that is more. In some aspects, the fixed pH setpoint is pH 6.91. In some aspects, lower integrated pH2 difference results in higher integrated viable cell density (IVCD) and higher titer at harvest. In some aspects, lower integrated pH2 difference further results in lower percent fucosylation.

In some embodiments, the rat hybridoma cells expressing the recombinant protein are cultured in a chemically defined and animal-derived component free (ADCF) culture medium.

In some embodiments, the recombinant protein's harvest titer is increased and/or fucosylation of the recombinant protein is decreased when the culture pH is 6.6 to 6.96 relative to a cell culture under the same culture conditions except that the culture pH is 6.60 to 6.8

In some embodiments, the method of making a recombinant protein in rat hybridoma cells further comprises controlling culture pCO₂ levels to less than about 300 mmHg. In some aspects, pCO₂ levels less than about 300 mmHg are facilitated by supplementing the cell culture with additional buffers, increasing the air sparge rate, increasing the dissolved oxygen (DO) setpoint, and/or decreasing the agitation rate.

In some embodiments, the method of making a recombinant protein in rat hybridoma cells further comprises an initial temperature set point of about 37° C., wherein said initial temperature set point is set on culture day 0 to culture day 1. In some aspects, the method of making a recombinant protein in rat hybridoma cells further comprises a second temperature set point of about 35° C., wherein said second temperature set point is set at the end of culture day 1 to culture day 3. In some aspects, the end of culture day one is 17 to 33 hours after the start of the cell culture.

In some embodiments, the method of making a recombinant protein in rat hybridoma cells further comprises a third temperature set point of about 32° C. to about 33° C., wherein said third temperature set point is set on culture day 3 and is maintained though harvest. In some aspects, the third temperature set point is 32.5° C.

In some embodiments, the cell culture in the methods disclosed herein comprises the following culture conditions: i). an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set on culture day 3 and is maintained though harvest; ii). a culture pH between about 6.5 to about 7.55; and iii). a culture pCO₂ less than about 300 mmHg. In some aspects, the yield of the recombinant protein made by the method disclosed herein is increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to a recombinant protein produced by a culturing process that does not employ the culture conditions in i)., ii)., and iii).

In some embodiments, the method of making a recombinant protein in rat hybridoma cells further comprises harvesting the recombinant protein produced by the rat hybridoma cells. In some aspects, the method further comprises purifying the recombinant protein by affinity chromatography and/or ion exchange chromatography. In some aspects, the affinity chromatography comprises protein A purification.

In some embodiments, the purified recombinant protein produced by the rat hybridoma cells is formulated into a pharmaceutically acceptable formulation.

In some embodiments, the quality of the purified recombinant protein produced by the methods disclosed herein is measured by SEC-HPLC, imaged capillary electrophoresis (ICIEF), and/or N-linked glycan analysis. In some aspects, the recombinant protein is a monoclonal antibody. In some aspects, the monoclonal antibody specifically binds an epitope of CD20.

In some embodiments, the monoclonal antibody is subject to CD20, FcγRIIIa-158V, and/or C1q binding assays. In some aspects, the monoclonal antibody has a relative potency of 82% to 138% in a cell-based CD20 binding activity bioassay compared to that of a commercial reference standard. In some aspects, CD20 binding is determined by the binding of an anti-CD20 antibody to the CD20 expressing human mantle cell lymphoma cell line, Jeko-1. In some aspects, the percentage of FcγRIIIa-158V binding of the monoclonal antibody is about 82% to about 130% with respect to a commercial reference standard for the binding assay. In some aspects, the percentage of FcγRIIIa-158V binding is determined by surface plasmon resonance (SPR). In some aspects, the monoclonal antibody has a relative potency of 86% to 117% in a C1q binding assay as measured by ELISA compared to a commercial reference standard. In some aspects, the monoclonal antibody has a relative potency of 88% to 113% in a C1q binding assay as measured by ELISA compared to a commercial reference standard. In some aspects, the monoclonal antibody has a relative potency of 74% to 127% in a cell-based complement dependent cytotoxicity (CDC) assay compared to that of a commercial reference standard. In some aspects, the monoclonal antibody produced has higher percent antibody-dependent cellular cytotoxicity (ADCC) activity relative to a monoclonal antibody produced by a culturing process that does not employ the culture conditions of i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set on culture day 3 and is maintained though harvest; ii) a culture pH between about 6.5 to about 7.55; and iii) a culture pCO₂ less than about 300 mmHg. In some aspects, the monoclonal antibody has a relative potency of 90% to 163% in a cell-based ADCC assay compared to a commercial reference standard. In some aspects, the monoclonal antibody has a relative potency of about 117% in a cell-based ADCC assay compared to a commercial reference standard.

In some embodiments, the monoclonal antibody produced by the methods disclosed herein comprises: a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some aspects, the monoclonal antibody comprises a heavy chain having at least 95% identity to an amino acid sequence set forth in SEQ ID NO:7 and a light chain having at least 95% identity to an amino acid sequence set forth in SEQ ID NO:8. In some aspects, the monoclonal antibody comprises a heavy chain having an amino acid sequence set forth in SEQ ID NO:7 and a light chain having an amino acid sequence set forth in SEQ ID NO:8. In some aspects, the monoclonal antibody comprises a heavy chain having an amino acid sequence set forth in SEQ ID NO:7 and a light chain having an amino acid sequence set forth in SEQ ID NO:9. In some aspects, the monoclonal antibody comprises a deletion of up to 5 N-terminal residues. In some aspects, the monoclonal antibody comprises a deletion of up to 10 N-terminal sequences.

In some embodiments, the cell culture is conducted in a bioreactor. In some aspects, the bioreactor is a commercial-scale bioreactor. In some aspects, the commercial-scale bioreactor is a 10,000 L, 15,000 L, 20,000 L, or 25,000 L bioreactor. In some aspects, the commercial-scale bioreactor is a 15,000 L bioreactor.

In some embodiments, the rat hybridoma cell that expresses the recombinant protein is a YB2/0 rat hybridoma cell.

Also provided herein is a recombinant protein made according to any of the method disclosed herein. In some aspects, the recombinant protein is a monoclonal antibody.

Also provided herein is a method of making a recombinant protein in a culture of rat hybridoma cells at a commercial-scale comprising the steps of: a) preparing and thawing a working rat hybridoma cell bank of the recombinant protein of interest; b) expanding a culture of the rat hybridoma cells from the cell bank by size and volume though a series of shake flasks (125 mL, 500 mL, 3 L, 3×3 L shake flasks, and 50 L cellbag) with a targeted seeding density of at least 0.30×10⁶ viable cells/mL; c) processing the cell culture through a series of seed bioreactors (120 L, 600 L, and 3,000 L) to further increase the volume and cell culture mass; d) inoculating the cell culture from the 3,000 L seed bioreactor into a commercial-scale production bioreactor; e) harvesting the cell culture supernatant from the commercial scale production bioreactor; f) clarifying the recovered cells by continuous centrifugation followed by depth filtration; g) purifying the recombinant protein by Protein A capture column chromatography; and h) inactivating viral agents by solvent detergent viral inactivation (SDVI). In some aspects, the commercial-scale production bioreactor is operated in fed-batch mode. In some aspects, a drilled hole (10) gas sparger with 4.0 mm orifice diameter is used in the commercial-scale production bioreactor.

In some embodiments, the method of commercial-scale production of a recombinant protein further comprises purification by cation exchange chromatography (CEX) and anion exchange chromatography (AEX). In some aspects, the method further comprises viral filtration (VF) to remove potential viruses. In some aspects, the method further comprises ultrafiltration/diafiltration (UFDF). In some aspects, the method further comprises preparing a bulk drug substance formulation comprising the recombinant protein, comprising adding polysorbate 80 in formulation buffer to prepare a bulk drug substance formulation. In some aspects, the method further comprises subjecting the bulk drug substance formulation to 0.2 μm filtration. In some aspects, the method further comprises filling a 6 L bag to a target fill volume of 5.50 L of the bulk drug substance formulation and storing the bulk drug substance formulation at ≤−35° C. In some aspects, the recombinant protein in the bulk drug substance formulation is formulated into a pharmaceutically acceptable formulation. In some aspects, the method further comprises testing unprocessed bulk harvest from the commercial-scale production bioreactor for microbial and viral adventitious agents. In some aspects, the method further comprises removing and/or inactivating the microbial and viral adventitious agents from the commercial-scale production bioreactor.

In some embodiments, in the method of commercial-scale production of a recombinant protein, the rat hybridoma cells are YB2/0 cells.

In some embodiments, the recombinant protein is a monoclonal antibody. In some aspects, the monoclonal antibody binds an epitope of CD20. In some aspects, the monoclonal antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some embodiments, the monoclonal antibody comprises a heavy chain having at least 95% identity to an amino acid sequence set forth in SEQ ID NO:7; and a light chain having at least 95% identity to an amino acid sequence set forth in SEQ ID NO: 8.

In some embodiments, the monoclonal antibody comprises a heavy chain having an amino acid sequence set forth in SEQ ID NO:7; and a light chain having an amino acid sequence set forth in SEQ ID NO:8.

In some embodiments, the monoclonal antibody comprises a heavy chain having an amino acid sequence set forth in SEQ ID NO:7; and a light chain having an amino acid sequence set forth in SEQ ID NO:9.

In some embodiments, the recombinant protein or monoclonal antibody produced by the methods disclosed herein comprises an N-glycan profile comprising one or both of the following: i) about 10 to 20% galactosylated glycans; and/or ii) about 20 to 40% fucosylated glycans. In some embodiments, the N-glycan profile comprises about 10 to 20% galactosylated glycans and about 20 to 40% (e.g., about 23 to 36%) fucosylated glycans. In some embodiments, the N-glycan profile comprises about 23% to about 36% fucosylated glycans. In some embodiments, the N-glycan profile comprises about 16% to about 18% galactosylated glycans. In some embodiments, the N-glycan profile comprises about 17% galactosylated glycans.

In some embodiments, the recombinant protein or monoclonal antibody produced by the methods disclosed herein comprises an N-glycan profile comprising at least about 10% bisecting N-glycans. In some embodiments, the N-glycan profile comprises about 12% to about 30% bisecting N-glycans. In some embodiments, the N-glycan profile comprises about 18% bisecting N-glycans.

In some embodiments, the recombinant protein or monoclonal antibody produced by the methods disclosed herein comprises an N-glycan profile comprising less than 5% sialylated glycans. In some embodiments, the N-glycan profile comprises less than 4%, 3%, 2.5%, 2%, 1%, or 0.5% sialylated glycans. In some embodiments, the N-glycan profile comprises no detectable amount of sialylated glycan.

In some embodiments, the recombinant protein or monoclonal antibody produced by the methods disclosed herein comprises an N-glycan profile comprising 0.1% to 1.5% Man5 N-glycan. In some embodiments, the N-glycan profile comprises 0.4% to 0.7% Man5 N-glycan. In some aspects, the N-glycan profile comprises about 0.6% Man5 N-glycan. In some embodiments, Man5 N-glycan is the only high mannose species in the N-glycan profile.

In some embodiments, the recombinant protein or monoclonal antibody is produced at a commercial-scale of about 10,000 L-about 25,000 L. In some embodiments, the commercial-scale is 15,000 L.

In some embodiments, the disclosed method yields a recombinant protein or monoclonal antibody harvest titer of about 0.5 g/L to about 1.5 g/L. In some embodiments, the harvest titer is about 1.0 g/L to about 1.5 g/L.

Also provided herein are rat hybridoma master cell bank (MCB) compositions and rat hybridoma working cell bank (WCB) compositions that can be used to produce the recombinant proteins (e.g., monoclonal antibodies) disclosed herein.

In some embodiments, the rat hybridoma MCB provided herein comprises a recombinant protein having at least two, at least three, at least four, at least five, at least six, at least seven, at least eight, at least nine, or all of the following parameters: i) peak viable cell density of about 11 to about 13×10⁶ cells/mL; ii) harvest titer of about 650 to about 720 mg/L; iii) percent fucosylation of about 30% to about 38%; iv) about 97% to about 99% monomers as detected by size exclusion chromatography (SEC); v) about 1.5% to about 2% dimers as detected by SEC; vi) undetectable to about 3% level of aggregates as detected by SEC; vii) undetectable to about 1% level of fragments as detected by SEC; viii) about 25% to about 30% acidic isoforms as detected by imaged capillary isoelectric focusing (iCIEF); ix) about 38% to about 49% main isoforms as detected by iCIEF; and/or x) about 20% to about 36% basic isoforms as detected by iCIEF.

In some embodiments, the rat hybridoma MCB provided herein comprises a recombinant protein having at least two, at least three, at least four, at least five, at least six, at least seven, at least eight, at least nine, or all of the following parameters: i) peak viable cell density of about 11 to about 13×10⁶ cells/mL; ii) harvest titer of about 650 to about 720 mg/L; iii) percent fucosylation of about 30% to about 38%; iv) about 97% to about 99% monomers as detected by size exclusion chromatography (SEC); v) about 1.5% to about 2% dimers as detected by SEC; vi) undetectable to about 3% level of aggregates as detected by SEC; vii) undetectable to about 1% level of fragments as detected by SEC; viii) about 25% to about 30% acidic isoforms as detected by imaged capillary isoelectric focusing (iCIEF); ix) about 38% to about 49% main isoforms as detected by iCIEF; and/or x) about 20% to about 36% basic isoforms as detected by iCIEF.

In some embodiments, the rat hybridoma WCB provided herein comprises a recombinant protein having at least two of the following parameters: i) peak viable cell density that is about 11 to about 28×10⁶ cells/mL; ii) harvest titer that is about 420 to about 1280 mg/L; iii) percent fucosylation of about 18% to about 40%; iv) about 97% to about 99% monomers as detected by size exclusion chromatography (SEC); v) about 1% to about 2% dimers as detected by SEC; vi) undetectable to about 2% level of aggregates as detected by SEC; vii) undetectable to about 1% level of fragments as detected by SEC; viii) about 19% to about 31% acidic isoforms as detected by imaged capillary isoelectric focusing (iCIEF); ix) about 34% to about 62% main isoforms as detected by iCIEF; and/or x) about 14% to about 38% basic isoforms as detected by iCIEF.

In some embodiments, the rat hybridoma WCB provided herein comprises a recombinant protein having at least two of the following parameters: i) peak viable cell density that is about 11 to about 28×10⁶ cells/mL; ii) harvest titer that is about 420 to about 1280 mg/L; iii) percent fucosylation of about 18% to about 40%; iv) about 97% to about 99% monomers as detected by size exclusion chromatography (SEC); v) about 1% to about 2% dimers as detected by SEC; vi) undetectable to about 2% level of aggregates as detected by SEC; vii) undetectable to about 1% level of fragments as detected by SEC; viii) about 19% to about 31% acidic isoforms as detected by imaged capillary isoelectric focusing (iCIEF); ix) about 34% to about 62% main isoforms as detected by iCIEF; and/or x) about 14% to about 38% basic isoforms as detected by iCIEF.

In some embodiments, the rat hybridoma cells in the MCB composition or the WCB composition are YB2/0 cells. In some embodiments, the recombinant protein to be produced from the MCB or WCB composition is a monoclonal antibody. In some embodiments, the monoclonal antibody is an anti-CD20 antibody.

In some embodiments, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some aspects, the anti-CD20 antibody comprises a heavy chain having at least 95% identity to an amino acid sequence set forth in SEQ ID NO:7; and a light chain having at least 95% identity to an amino acid sequence set forth in SEQ ID NO: 8. In some embodiments, the anti-CD20 antibody comprises a heavy chain having an amino acid sequence set forth in SEQ ID NO:7; and a light chain having an amino acid sequence set forth in SEQ ID NO:8. In some embodiments, the anti-CD20 antibody comprises a heavy chain having an amino acid sequence set forth in SEQ ID NO:7; and a light chain having an amino acid sequence set forth in SEQ ID NO:9.

Also provided is a method of making a recombinant protein by using the MCB or WCB composition, as disclosed herein. In some aspects, the recombinant protein is a monoclonal antibody. In some embodiments, the monoclonal antibody is an anti-CD20 antibody. In some embodiments, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some embodiments, the anti-CD20 antibody comprises a heavy chain having at least 95% identity to an amino acid sequence set forth in SEQ ID NO:7; and a light chain having at least 95% identity to an amino acid sequence set forth in SEQ ID NO: 8. In some embodiments, the anti-CD20 antibody comprises a heavy chain having an amino acid sequence set forth in SEQ ID NO:7; and a light chain having an amino acid sequence set forth in SEQ ID NO:8. In some aspects, the anti-CD20 antibody comprises a heavy chain having an amino acid sequence set forth in SEQ ID NO:7; and a light chain having an amino acid sequence set forth in SEQ ID NO:9.

### BRIEF DESCRIPTION OF THE DRAWINGS

FIG. 1 depicts nitrogen-linked (N-linked) protein glycosylation species (“N-glycans”) that may be covalently attached to the recombinant proteins described herein and that can be released by enzymatic deglycosylation and quantified. N-glycans typically comprise five principal sugars: N-acetyl-D-glucosamine (GlcNAc); mannose (Man); galactose (Gal); fucose (Fuc); and N-Acetylneuraminic acid (NANA), as shown.

FIG. 2 is a bar graph showing the impact of different cell culture media on process performance—Cell Growth (as measured by IVCD), as described in Example 1. The numbers 1105-1152 along the X-axis reflect the different cell culture media. Table 5 provides the name of the cell culture media associated with a given number.

FIG. 3 is a bar graph showing the impact of different cell culture media on process performance—Cell Viability, as described in Example 1. The numbers 1105-1152 along the X-axis reflect the different cell culture media. Table 5 provides the name of the cell culture media associated with a given number.

FIG. 4 is a bar graph showing the impact of different cell culture media on process performance—Harvest Titer, as described in Example 1.

FIG. 5 is a bar graph showing the impact of different cell culture media on process performance: % Fucosylation, as described in Example 1.

FIG. 6 is a scatter plot showing the impact of cell culture media change on % fucosylation, as described in Example 1.

FIG. 7 is a scatter plot showing the impact of cell culture media change on FcγRIIIa-158V binding, as described in Example 1.

FIG. 8 is a scatter plot showing the impact of cell culture media change on CD20 binding, as described in Example 1.

FIG. 9 is a scatter plot showing the impact of cell culture media change on ADCC activity, as described in Example 1.

FIG. 10 is a scatter plot showing the impact of cell culture media change on C1q binding, as described in Example 1.

FIG. 11 is a scatter plot showing the impact of cell culture media change on CDC activity, as described in Example 1.

FIG. 12 is a scatter plot showing cumulative cell growth (IVCD) versus cumulative pCO₂ exposure, as described in Example 2.

FIG. 13 is a scatter plot showing harvest titer versus cumulative pCO₂ exposure.

FIG. 14 is a graph showing viable cell density (VCD) results from cell cultures evaluating different pH control ranges (post-shift), as described in Example 2.

FIG. 15 is a graph showing cell viability (%) results from cell cultures evaluating different pH control ranges (post-shift), as described in Example 2.

FIG. 16 is a graph showing in-process glucose results from cell cultures evaluating different pH control ranges (post-shift), as described in Example 2.

FIG. 17 is a graph showing in-process lactate results from cell cultures evaluating different pH control ranges (post-shift), as described in Example 2

FIG. 18 is a graph showing in-process pCO₂ results from cell cultures evaluating different pH control ranges (post-shift), as described in Example 2.

FIG. 19 is a graph showing in-process titer results from cell cultures evaluating different pH control ranges (post-shift), as described in Example 2.

FIG. 20 depicts the map of expression vector, HK463-25, containing the immunoglobulin heavy and light chain cDNA sequences of source anti-CD20 antibody, TG-1101, described herein.

FIG. 21 is a graph depicting schematically the process parameter, “integrated pH2 difference,” as described in Example 5.

FIG. 22 is a graph depicting the variability in post-shift pH (“pH2”) in typical Process C2 cultures, as described in Example 5.

FIG. 23 is a graph depicting the impact of integrated pH2 difference on IVCD, as described in Example 5.

FIG. 24 is a graph depicting the impact of integrated pH2 difference on harvest titer, as described in Example 5.

FIG. 25 is a graph depicting the impact of integrated pH2 difference on percent fucosylation, as described in Example 5.

FIG. 26 illustrates the glycosylation profile of a sample of anti-CD20 antibodies, as described in Example 6.

### DETAILED DESCRIPTION OF THE DISCLOSURE

Provided herein are methods for making recombinant proteins (e.g., monoclonal antibodies) in an uncommonly-used mammalian expression system—rat hybridoma cells, e.g., YB2/0. Also provided are methods for increasing product titer and improving reproducibility, homogeneity, product quality (e.g, low percent fucosylation), and functional activity of recombinant proteins by modifying cell culture process parameters and/or cell culture media. Also provided are methods for commercial-scale production (e.g., 10,000 L, 15,000 L, 20,000 L, 25,000 L) of recombinant proteins (e.g., monoclonal antibodies) in rat hybridoma cells that retain high protein quality and functional activity. Also provided herein are compositions made in accordance with the presently disclosed methods, including pharmaceutical compositions.

In some embodiments, the methods of the present disclosure are used to produce an anti-CD20 antibody at a commercial-scale (e.g., 10,000 L-25,000 L). In some aspects, the anti-CD20 antibody made by the manufacturing process described herein has a unique glycosylation profile, which will be described further below. Without being bound by theory, the relative distribution of the various N-glycans, or individual sugar residues present in those N-glycans, can determine the biological and clinical properties of the anti-CD20 antibody. See, co-pending and commonly owned U.S. Provisional Appl. No. 63/347,852, entitled “Anti-CD20 Antibody Compositions,” filed Jun. 1, 2022, which is incorporated by reference herein in its entirety.

### ABBREVIATIONS

Table 1 provides a list of abbreviations used in the present disclosure.

TABLE 1
List of abbreviations

| | |
|---|---|
| ADCC | Antibody-dependent cellular cytotoxicity |
| CDC | Complement dependent cytotoxicity |
| CHO | Chinese hamster ovary |
| CQA | Critical quality attributes |
| DO | Dissolved oxygen |
| FcγRIIIa-158V | Fc gamma receptor 3a, higher affinity allele |
| Fuc | Fucose |
| Gal | Galactose |
| GlcNAc | N-acetylglucosamine |
| GMP | Good manufacturing practices |
| HPLC | High performance liquid chromatography |
| iCIEF | Imaging capillary isoelectric focusing |
| IgG | Immunoglobulin G |
| IVCD | Integrated viable cell density (cell density |
| | integrated over the culture duration) |
| Man | Mannose |
| NANA | N-acetylneuraminic acid |
| pCO₂ | Dissolved carbon dioxide |
| PK | Pharmacokinetics |
| PTM | Post-translational modifications |
| SEC | Size exclusion chromatography |
| VCD | Viable cell density |
| YB2/0 | Rat hybridoma cell line |
| AR | Acceptable Range |
| NOR | Normal Operating Range |

### Definitions

In order that the present disclosure may be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Tus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “inoculating,” “inoculation,” or “seeding” refer to the process of providing a cell culture to a bioreactor (e.g., a production bioreactor) or another vessel. In some aspects, the cells have been propagated previously in another bioreactor or vessel. In some aspects, the cells have been frozen and thawed immediately prior to providing them to the bioreactor or vessel.

As used herein, the term “cell culture” refers to the suspension of growing mammalian cells (e.g., rat hybridoma cells) in culture medium in accordance with the present disclosure. As will be apparent from context, the term “cell culture” or “in a cell culture” can also refer to the closed container, vessel, or bioreactor that is used for growing mammalian cells. See also “bioreactor.”

As used herein, the term “bioreactor” refers to a commercial-scale, large-scale, small-scale, or micro-scale vessel that is used to grow mammalian cells (e.g., rat hybridoma cells) in accordance with the methods of the present disclosure. Bioreactors permit various cell culture parameters to be “controlled” during the cell culture process including, but not limited to, the circulation loop flow, pH, the temperature, the overpressure, and/or the medium perfusion rate. Bioreactors include, for example, commercially available bioreactors, stirred tank bioreactors, airlift bioreactors, bubble column bioreactors, hollow fiber bioreactors, fluidized bed bioreactors, membrane bioreactors, classical fermenters, bench-top bioreactors, 10-15 ml and 250 mL microscale bioreactors (e.g., (ambr) (Sartorius)), and cell culture perfusion systems, as well as disposable or single-use bioreactors.

The bioreactor can be any size that is useful for culturing cells at a desirable scale in accordance with the methods of the present disclosure. For example, in some aspects, a bioreactor employed in the methods of the present disclosure may range from about 250 ml to about 25,000 liters. In some aspects, the bioreactor has a capacity of about 0.1, 0.5, 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 340, 350, 360, 370, 380, 390, 400, 410, 420, 430, 440, 450, 460, 470, 480, 490, 500, 550, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,0000, 13,000, 14,000, 15,000, 20,000 liters, 25,000 liters or more, or any intermediate volume.

In some aspects, recombinant protein production is at a commercial-scale. In certain aspects, the commercial-scale bioreactor can be 10,000 L to 25,000 L. Thus, as used herein, the term “commercial-scale production” of a recombinant protein (e.g., an antibody) refers to production in an at least 10,000 L, at least 15,000 L, at least 20,000 L, or at least 25,000 L bioreactor. In some aspects, the commercial-scale bioreactor is 15,000 L.

A suitable bioreactor may be constructed of any material that is suitable for holding cell cultures under the culture conditions of the present disclosure and is conducive to cell growth and viability. For example, a bioreactor employed in the methods of the present disclosure can be made of glass, plastic, or metal. Suitable bioreactors are known in the art and commercially available.

As used herein, the term “production bioreactor” refers to any vessel made of glass, plastic, or metal between 15 mL to 25,000 L that supports a culture environment conducive towards cell growth. For example, in Example 3, the production bioreactor is a 15,000 L stainless steel bioreactor. In some aspects, such as described in Example 3, the term “production bioreactor” (or N culture vessel) refers to the final bioreactor in a series of vessels of increasing scale (such as N-4, N-3, N-2, and N-1 vessels or “seed bioreactors”), which is inoculated with the cell culture (also called “the seed culture”) from the immediately preceding N-1 vessel having high viable cell density, and where the cells will continue to grow until harvest. In some aspects, the cells in the cell culture are harvested from the production bioreactor. (See, e.g., Example 3).

As used herein, the term “seed bioreactor” refers to any cell culture vessel used prior to transfer of the cell culture to the production bioreactor. (See, e.g., Example 3).

As used herein, the term “control,” “controlled,” or “controlling” refers to the ability of a protein structure/function parameter to be purposely increased, decreased, or maintained the same. In the methods of the present disclosure, culture pH and temperature are examples of cell culture parameters that can be controlled.

As used herein, the term “cell,” refers to mammalian cells, cultured cells, host cells, recombinant cells, and recombinant host cells. Such cells are generally cell lines obtained or derived from mammalian tissues which are able to grow and survive when placed in culture media containing appropriate nutrients and/or growth factors. The cells utilized in the methods of the present disclosure are mammalian rat hybridoma cells (e.g., YB2/0) that can express and secrete, or that can be molecularly engineered to express and secrete, large quantities of a recombinant protein (e.g., antibody) into the culture medium.

Generally, the “cells,” as described herein, are cultured for a consecutive number of “culture days” (or, alternatively, “process days”) until they are harvested. In some aspects, the number of “culture days” can include 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or more.

As used herein, “culture day 1” refers to about 12.0 hours to about 35.9 hours post-inoculation of the cells in the seed culture to the production bioreactor. As used herein, “culture day 2” refers to about 36.0 hours to about 59.9 hours post-inoculation of the cells in the seed culture to the production bioreactor. As used herein, “culture day 3” refers to about 60.0 hours to about 83.9 hours post-inoculation of the cells in the seed culture to the production bioreactor.

As used herein, the term “culture condition(s)” refers to the cell culture conditions, e.g., pH, temperature, pCO₂, at various levels described herein, or shifts in those levels, which result in expression of a recombinant protein (e.g., antibody) having increased growth, titer, cell density, cell viability, or improved product quality relative to a cell culture that was not cultured under a particular culture condition(s).

As used herein, the terms “media,” “cell culture media,” “basal media,” and “culture media,” including grammatical variations thereof, are used interchangeably, and refer to the physiochemical, nutritional, and hormonal environment in which mammalian cells (e.g., rat hybridoma cells) can grow in culture and express a recombinant protein of interest (e.g., monoclonal antibody). Exemplary cell culture media are described below.

As used herein, the term “set” or “setting” (e.g., “set” a pH) refers to the inputting of a particular value(s) for a process parameter (such as, e.g., temperature, pH) into the bioreactor or other cell culture vessel control system. See also “set point.”

As used herein, “set point” refers to the setting of a condition on a bioreactor or other cell culture vessel used to grow cells and/or produce protein product, unless otherwise indicated. In some aspects, a set point is set on a production bioreactor. A set point can be established at the outset of cell culture and/or re-set to a different set point during cell culture. For example, in some aspects, a set point can be a “pH set point.” In some aspects, a set point is a “temperature set point.” In some aspects, the set point can be maintained throughout the cell culturing method. In other aspects, the set point can be maintained until a different set point is set. In other aspects, the set point can be changed to another set point.

As used herein, “temperature set point” refers to the temperature setting of a bioreactor (e.g., production bioreactor) or other cell culture processing vessel used to grow cells and/or produce protein product. A temperature set point can be established at the outset of cell culture in the production bioreactor, where it can also be referred to as an “initial temperature set point.” Subsequent changes in temperature during cell culture after the initial temperature set point are referred to as a second temperature set point, or subsequently, a third temperature set point. The last temperature set point before harvest can also be referred to as a “final temperature set point.” In some aspects, a process can comprise an initial temperature set point, a second temperature set point, and a third (and final) temperature set point.

In some aspects, the “initial temperature set point” is set on culture day 0 to culture day 1. In some aspects, a “second temperature set point” is set at the end of culture day 1 to culture day 3. In some aspects, a “third temperature set point” is set on culture day 3 and is maintained though harvest.

As used herein, the term “pH” refers to the culture pH in the production bioreactor, post-inoculation of the cells in the seed cultures to the production bioreactor.

As used herein, the term “pH2” refers to the culture pH in the production bioreactor after the pH shift on culture day 3 (about 62 to 77 hours, preferably 72 hours) to a lower pH, from the culture pH post-inoculation (“pH1”).

As used herein, the term “integrated pH2 difference” is a calculated pH parameter that refers to the cumulative culture time that pH is allowed to drop below a fixed pH setpoint, as well as the cumulative magnitude of that drop, after the pH is lowered on culture day 3. The integrated pH2 difference increases when culture pH drops below a fixed pH setpoint for any unit measure of time. In some aspects, the integrated pH2 difference of a cell culture relative to a fixed pH setpoint (e.g., 6.91) is less, relative to a cell culture with an “integrated pH2 difference” that is more. In some aspects, lower integrated pH2 difference results in higher integrated viable cell density (IVCD), higher titer at harvest, and lower percent fucosylation.

As used herein, the term “polynucleotide” or “nucleic acid” refers to a polymeric form of nucleotides of any length, including ribonucleotides and deoxyribonucleotides. This term refers to the primary structure of the molecule. Thus, the term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides.

As used herein, the term “protein,” peptide, and “polypeptide” are used interchangeably to refer to an amino acid polymer of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, a “recombinant protein” refers to a polypeptide or protein produced via recombinant DNA technology. Recombinantly produced polypeptides and proteins, as disclosed herein, are expressed in engineered host cells (such as, e.g., rat hybridoma cells). The term “protein” is intended to include glycoproteins.

As used herein, the term “percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, the term “expression vector” refers to any nucleic acid construct that contains the necessary elements (e.g., promoter, enhancer) for the transcription and translation of an inserted coding sequence of a polypeptide of interest when introduced into a host cell (e.g., rat hybridoma cells). Expression vectors can include plasmids, phagemids, viruses, and derivatives thereof. Expression vectors of the disclosure can include polynucleotides encoding a recombinant protein (e.g., monoclonal antibody).

As used herein, the term “glycoprotein” refers to a protein that is modified by the addition of one or more carbohydrate moieties, e.g., a polysaccharide or an oligosaccharide, that is attached to the protein via an oxygen-containing or a nitrogen-containing side chain of an amino acid residue, e.g., a serine or threonine residue (“O-linked”) or an asparagine residue (“N-linked”).

As used herein, the term “glycan” refers to a polysaccharide or an oligosaccharide, e.g., a polymer or oligomer, comprised of monosaccharide residues.

As used herein, the term “upstream process,” in the context of protein, e.g., antibody, preparation, refers to activities involving the production and collection of proteins (e.g. antibodies) from cells (e.g., during cell culture of a recombinant protein).

As used herein, the term “downstream process,” in the context of protein, e.g., antibody, preparation, refers to one or more techniques used after the upstream process technologies in order to purify the protein, e.g., antibody, of interest. For example, downstream process technology includes purification of the protein product, using, for example, affinity chromatography, including Protein A affinity chromatography, ion exchange chromatography, such as anion or cation exchange chromatography, viral filtration, depth filtration, ultrafiltration, diafiltration, and centrifugation.

As used herein, the term “glycosylation” in connection with a recombinant glycoprotein refers to the addition of complex oligosaccharide structures to a protein at specific sites within the polypeptide chain. Glycosylation of proteins and the subsequent processing of the added carbohydrates can affect protein folding and structure, protein stability, including protein half-life, and functional properties of a protein. Protein glycosylation can be divided into two classes by virtue of the sequence context where the modification occurs, O-linked glycosylation and N-linked glycosylation. O-linked polysaccharides are linked to a hydroxyl group, usually to the hydroxyl group of either a serine or a threonine residue. O-glycans are not added to every serine and threonine residue. O-linked oligosaccharides are usually mono or biantennary, i.e., they comprise one or at most two branches (antennas), and comprise from one to four different kinds of sugar residues, which are added one by one. N-linked polysaccharides are attached to the amide nitrogen of an asparagine. Only asparagines that are part of one of two tripeptide sequences, either asparagine-X-serine or asparagine-X-threonine (where X is any amino acid except proline), are targets for glycosylation. N-linked oligosaccharides can have from one to four branches referred to as mono-, bi-, tri-tetraantennary. The structures of and sugar residues found in N- and O-linked oligosaccharides are different. Despite that difference, the terminal residue on each branch of both N- and O-linked polysaccharides can be modified by a sialic acid residue. Sialic acid is a common name for a family of unique nine-carbon monosaccharides, which can be linked to other oligosaccharides. The two main types of sialyl residues found in biopharmaceuticals produced in mammalian expression systems are N-acetyl-neuraminic acid (NANA) and N-glycolyl-neuraminic acid (NGNA).

As used herein, the term “sialylation” refers to the addition of a sialic acid (S) residue, for example, N-glycans: G1FS1, G2S1, G2FS1, G2FBS1, G2S2, G2FS2, G2FBS2, to a recombinant glycoprotein.

As used herein, the term “fucosylation” or “protein fucosylation” refers to the addition of a fucose (F) residue, for example, N-glycans: G0F-GN, G0F, G0FB, G1F, G1FB, and G2F, to a recombinant glycoprotein.

As used herein, the term “galactosylation” refers to the addition of a galactose (gal) residue, for example, N-glycans: G1, G2, G1F, G1FB, G2F, to a recombinant glycoprotein.

As used herein, the term “percent fucosylation” or “% fucosylation” refers to the percentage of N-glycans that possess a fucose sugar amongst all N-glycans. Similarly, the term “percent galactosylation” or “% galactosylation” refers to the percentage of N-glycans that possess a galactose sugar amongst all N-glycans. Percent fucosylation, for example, is calculated by subjecting a sample or a population of anti-CD20 antibody proteins to enzymatic deglycosylation so that all N-glycans are cleaved from the core. The resulting N-glycans can subsequently be analyzed, e.g., by mass spectrometry. The percent of fucosylated N-glycans is the percent of fucosylated N-glycans among N-glycans that were cleaved using the enzymatic digest.

As used herein, the term “cell density” refers to the number of cells in a given volume of medium. Cell density can be monitored by any technique known in the art, including, but not limited to, extracting samples from a culture and analyzing the cells under a microscope, using a commercially available cell counting device, or by using a commercially available suitable probe introduced into the bioreactor itself (or into a loop through which the medium and suspended cells are passed and then returned to the bioreactor).

As used herein, the term “integral viable cell density” or “IVCD” refers to viable cell density integrated over the duration of the cell culture.

As used herein, the term “viable cell density” or “VCD” refers to the number of live cells present in a given volume of medium under a given set of experimental conditions.

As used herein, the term “cell viability” refers to the ability of cells in cell culture to survive under a given set of conditions or experimental variations. The term as used herein also refers to the percentage (%) of cells that are alive at a particular time in relation to the total number of cells (e.g., living and dead) in the culture at that time.

As used herein, the term “shift” refers to a modulation or change in a particular cell culture parameter (e.g., pH shift, temperature shift).

As used herein, the term “post-shift” (when preceding a particular numerical value or percentage) indicates that the numerical value or percentage of a particular level (e.g., glucose, lactate, % fucosylation, % sialylation, titer), or activity (e.g., binding or effector function) of the cell culture or the expressed recombinant protein (e.g., antibody), was taken at any time after the shift in a particular cell culture parameter.

As used herein, the “initial growth phase” of a cell culture refers to culture days 0 to 2 (i.e., days 0, 1, and/or 2), during which the cells (e.g., YB2/0) begin to grow. In the “initial growth phase,” the amounts of recombinant protein expressed by the cell culture are significantly lower than in later days in the protein generation phase (i.e., culture days 3 to harvest).

As used herein, the “protein generation phase” of a cell culture refers to culture days 3 through harvest, during which the rat hybridoma cells (e.g., YB2/0) produce recombinant protein at significantly higher amounts than in the initial growth phase, such that titers can be measured.

As used herein, the term “harvest” refers to the point of time in the mammalian cell culture process when cells containing the recombinant protein are separated and removed from the cell culture media and subject to additional processing, such as, e.g., centrifugation, filtration, or purification. In some aspects, harvest of the cells will occur on process day 12 or 13 of the cell culture, or when cell viability drops below 200/a, whichever comes first. See also, “harvest titer.”

As used herein, the term “harvest material” refers to the cells containing the recombinant protein that are recovered from the production bioreactor at “harvest.”

As used herein, the term “Master Cell Bank” or “MCB” refers to the cell bank generated under GMP conditions from cells expanded from the TG-1101-producing YB2/0 cell line, described herein.

As used herein, the term “Working Cell Bank” or “WCB” refers to the cell bank generated under GMP conditions from cells expanded from the MCB.

As used herein, the term “bulk drug substance formulation” refers to the final formulated material at the conclusion of the purification unit operations of the manufacturing process, described herein.

As used herein, the term “unprocessed bulk” refers to harvest material from the production bioreactor before clarification.

As used herein, the term “harvest clarification” refers to the primary purification stage for removal of cells, particulates, and impurities from the production bioreactor harvest material. See, e.g., Example 3. After the harvest clarification stage, the clarified harvest can be stored for, e.g., 24 hours, 2 days, 3 days, 4 days, 5 days, 6 days, 7 days, 8 days, 9 days, 10 days, 11 days, or ≤11 days.

As used herein, the term “single batch,” in the context of recombinant proteins (e.g., anti-CD20 antibodies), refers to a composition derived from a single production or run from a single bioreactor of a specified volume. For example, the anti-CD20 antibodies obtained from a single run of a 15,000 L bioreactor can be referred to as a single batch. In some aspects, the anti-CD20 antibodies are present in such a single batch at a concentration of at least 10 mg/ml; 15 mg/ml; 20 mg/ml; 25 mg/ml; or at least 30 mg/ml. In some aspects, anti-CD20 antibodies are present in such a single batch at a concentration of at between 10 to 35 mg/ml; 10 to 30 mg/ml; 10 to 25 mg/ml; 10 to 20 mg/ml; 10 to 15 mg/ml; 15 to 35 mg/ml; 15 to 30 mg/ml; 15 to 25 mg/ml; 15 to 20 mg/ml; 20 to 35 mg/ml; 20 to 30 mg/ml; 20 to 25 mg/ml; 25 to 35 mg/ml; or 25 to 30 mg/ml. In some aspects, anti-CD20 antibodies are present in such a single batch at a concentration of about 15 mg/ml; about 20 mg/ml; about 25 mg/ml; about 30 mg/ml; or about 35 mg/ml. In some aspects, the amount of total protein is quantified by spectrophotometry. In some aspects, the amount of total protein is quantified by spectrophotometric absorbance at 280 nm.

As used herein, the term “adventitious agents” refers to microorganisms (e.g., bacteria, fungi, viruses, mycoplasma) capable of posing a risk to human health, which are unintentionally introduced into the manufacturing process of a biological medicinal product.

As used herein, the term “titer” refers to the total amount of recombinantly expressed protein (e.g., antibody) produced by a cell culture, divided by a given amount of medium volume. The term “titer” refers to a concentration and is typically expressed in units of milligrams (mg) of protein per milliliter (mL) or liter (L) of medium. In some aspects, the methods of the present disclosure may substantially increase protein product titer, as compared to protein product titers produced from other cell culture methods known in the art, or from cell culture methods that do not employ the culture conditions described herein.

As used herein, the term “harvest titer” refers to the total amount of protein (e.g., antibody) produced by the cell culture at the time of harvest of the cells from the cell culture, cell culture vessel, or bioreactor. In some aspects, harvest of the cells will occur on process day 12 or 13 of the cell culture, or when cell viability drops below 20/a, whichever comes first.

As used herein, the term “antibody” or “Ab” shall include, without limitation, a glycoprotein immunoglobulin that binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2, and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FRs). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG, and IgM. IgG subclasses are also well known to those in the art and include but are not limited to human IgG1, IgG2, IgG3, and IgG4. “Isotype” refers to the antibody class or subclass (e.g., IgM or IgG1) that is encoded by the heavy chain constant region genes.

The term “antibody” includes, by way of example, monoclonal antibodies; polyclonal antibodies; chimeric, and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

As used herein, the term “epitope” refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from L1CAM) are tested for reactivity with a given antibody (e.g., anti-L1CAM antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)). In some aspects, an antibody can bind to more than one epitope (e.g., TG-1101).

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “acceptable range” or “AR” refers to the range of values for certain process parameters (e.g., pH), whereby control within the values ensures that process performance as well as product quality attributes meet their specifications. Excursions from an “acceptable range” may lead to formal root cause analysis and impact assessment.

### Methods of Making Recombinant Proteins (e.g., Monoclonal Antibodies) in Rat Hybridoma Cells

In one aspect, the present disclosure provides a method of making a recombinant protein (e.g., monoclonal antibody) in rat hybridoma cells comprising culturing the cells in a cell culture having a culture pH of about 6.5 to about 7.55, wherein the rat hybridoma cells comprise an expression vector comprising a polynucleotide encoding the recombinant protein. In some aspects, the culture pH is about 6.5 to about 7.0. In some aspects, the culture pH of about 6.5 to about 7.0 is set on culture day 2 of the cell culture. In some aspects, the culture pH of about 6.5 to about 7.0 is set on culture day 3 of the cell culture. In some aspects, the culture pH is about 7.0 to about 7.55. In some aspects, the culture pH is set to about 7.0 to about 7.55 on culture days 0 to 3 of the cell culture.

In some aspects, the culture pH is lowered on day 2 or day 3 of the cell culture to about 6.5 to about 7.0. In some aspects, the culture pH is lowered on day 3 of the cell culture. In some aspects, the culture pH of about 6.5 to about 7.0 is maintained from culture day 3 until harvest of the cell culture.

“Integrated pH2 difference” is a calculated pH parameter that refers to the cumulative culture time that pH is allowed to drop below a fixed pH setpoint, as well as the cumulative magnitude of that drop, after the pH is lowered on culture day 3. The integrated pH2 difference increases when culture pH drops below a fixed pH setpoint for any unit measure of time. In some aspects, the integrated pH2 difference of a cell culture relative to a fixed pH setpoint is less, relative to a cell culture with an “integrated pH2 difference” that is more. In some aspects, the fixed pH setpoint is pH 6.91. In some aspects, lower integrated pH2 difference results in higher integrated viable cell density (IVCD) and higher titer at harvest. In some aspects, lower integrated pH2 difference further results in lower percent fucosylation.

In some aspects, the rat hybridoma cells expressing the recombinant protein are cultured in a chemically defined and animal-derived component-free (AFCP) culture medium. In some aspects, the basal culture medium is CDM4Mab® (Cytiva) and the feed medium is BalanCD CHO Feed 4® (Irvine Scientific).

In some aspects, the harvest titer of the recombinant protein is increased and/or fucosylation of the recombinant protein is decreased when the culture pH is 6.6 to 6.96 relative to a cell culture under the same culture conditions except that the culture pH is 6.60 to 6.8.

In some aspects, culture pCO₂ levels in the cell culture are controlled to less than about 300 mmHg. In some aspects, pCO₂ levels less than about 300 mmHg are facilitated by supplementing the cell culture with additional buffers, increasing the air sparge rate, increasing the dissolved oxygen (DO) setpoint, and/or decreasing the agitation rate.

In some aspects, the culture conditions further comprise an initial temperature set point of about 37° C., wherein said initial temperature set point is set on culture day 0 to culture day 1. In some aspects, the culture conditions further comprise a second temperature set point of about 35° C., wherein said second temperature set point is set at the end of culture day 1 to culture day 3. In some aspects, the “end of culture day 1” is 17 to 33 hours after the start of the cell culture. In some aspects, the culture conditions further comprise a third temperature set point of about 32° C. to about 33° C., wherein said third temperature set point is set on culture day 3 and is maintained though harvest. In some aspects, the third temperature set point is 32.5° C.

In some aspects, the present disclosure relates to a method of making a recombinant protein, wherein the cell culture comprises the following culture conditions: i). an initial temperature set point of about 37° C., wherein the initial temperature set point is set on culture day 0 to culture day 1; a second temperature set point of about 35° C., wherein the second temperature set point is set at the end of culture day 1 to culture day 3; and a third temperature set point of about 32.5° C., wherein the third temperature set point is set on culture day 3 and maintained though harvest; ii). a culture pH between about 6.5 to about 7.55; and iii) a culture pCO₂ less than about 300 mmHg. In some aspects, the “end of culture day 1” is 17 to 33 hours after the start of the cell culture.

In some aspects, the yield of the recombinant protein is increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to a recombinant protein produced by a culturing process that does not employ i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set culture day 3 and is maintained though harvest; ii) a culture pH between about 6.5 to about 7.55; and iii) a culture pCO₂ less than about 300 mmHg.

In some aspects, the presently disclosed method further comprises harvesting the recombinant protein produced by the rat hybridoma cell. In some aspects, purifying the recombinant protein is by affinity chromatography and/or ion exchange chromatography. In some aspects, the affinity chromatography comprises protein A purification.

In some aspects, the purified recombinant protein produced by the rat hybridoma cell is formulated into a pharmaceutically acceptable formulation. In some aspects, the quality of the purified recombinant protein is measured by SEC-HPLC, imaged capillary electrophoresis (ICIEF), and/or N-linked glycan analysis.

In some aspects, the recombinant protein is a monoclonal antibody. In some aspects, the monoclonal antibody specifically binds an epitope of CD20. In some aspects, the monoclonal antibody is subjected to various assays in order to assess potency and biological activity. In some aspects, wherein the monoclonal antibody is subject to CD20, FcγRIIIa-158V, and/or C1q binding assays. The results of these bioassays will be discussed below.

### Rat Hybridoma Cells

Many mammalian cells or cell types susceptible to cell culture, and to expression of polypeptides, are known in the art, such as, e.g., BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol, 36:59 (1977)); baby hamster kidney cells (BHK, ATCC CCL 10); Chinese hamster ovary cells ±DHFR (CHO, Urlaub and Chasin, Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL5 1); TRI cells (Mather et al., Annals N.Y. Acad. Sci. 383:44-68 (1982)); MRC 5 cells; FS4 cells; and a human hepatoma line (Hep G2).

In some aspects, the mammalian cells used to culture and express recombinant proteins (e.g., monoclonal antibodies) in accordance with the methods of the present disclosure are rat hybridoma cells. Exemplary rat hybridoma cells include YB2/0, IR983F, IR2, and IR162.

In some aspects, the rat hybridoma cells used in the methods of the present disclosure are YB2/0 rat hybridoma cells (ATCC CRL 1662).

### Recombinant Proteins (e.g., Therapeutic Antibodies)

Any protein or polypeptide that is expressible in rat hybridoma cells may be produced as a recombinant protein in accordance with the methods of the present disclosure. In one aspect, the recombinant protein that may be produced from the rat hybridoma cells in accordance with the present disclosure includes, for example, any pharmaceutically or commercially relevant antibody, enzyme, receptor, hormone, regulatory factor, antigen, or binding agent.

Around the world, at least 570 therapeutic monoclonal antibodies (mAbs) have been studied in clinical trials by commercial companies, and as of December 2019, 79 therapeutic mAbs have been approved by the U.S. FDA and are currently on the market, including 30 mAbs for the treatment of cancer. And there is still significant growth potential. See, Lu, R.-M. et al., Journal of Biomedical Science 27:1 (2020). The top 10 best-selling therapeutic antibody drugs in 2018 include Rituximab (Rituxan): anti-CD20 for Non-Hodgkin lymphoma; Trastuzumab (Herceptin): anti-HER2 for breast cancer; Infliximab (Remicade): anti-TNFα for Crohn's disease; Pembrolizumab (Keytruda): anti-PD-1 for melanoma; Nivolumab (Opdivo): anti-PD-1 for melanoma and non-small cell lung cancer (NSCLC); Ustekinumab (Stelara): anti-IL-12/23 for Psoriasis; Eculizumab (Soliris): anti-C5 for Paroxysmal nocturnal hemoglobinuria; Bevacizumab (Avastin): anti-VEGF for colorectal cancer (CRC); Omalizumab (Xolair): anti-IgE for asthma; and Adalimumab (Humira): anti-TNFα for rheumatoid arthritis (RA). Id.

Given the large number of antibodies currently in use or under investigation as therapeutic agents, the production of antibodies, in accordance with the methods of the present disclosure, is a preferred aspect. Any antibody, or an antigen-binding fragment thereof, that can be produced in a rat hybridoma cells may be used in accordance with the present disclosure. In some aspects, the antibody to be produced is a monoclonal antibody, or an antigen-binding fragment thereof. In some aspects, the antibody is a polyclonal antibody, or an antigen-binding fragment thereof. In some aspects, the antibody is a chimeric antibody. In some aspects, the antibody is a humanized antibody. In some aspects, the antibody is a human antibody.

In some aspects, the monoclonal, polyclonal, chimeric, or humanized antibodies described above may contain amino acid residues that do not naturally occur in any antibody in any species in nature. These foreign residues can be utilized, for example, to confer novel or modified specificity, affinity or effector function on the monoclonal, chimeric or humanized antibody.

### Anti-CD20 Antibodies

In one aspect, the monoclonal antibody produced according to the presently disclosed methods binds to CD20 or an epitope of CD20. The term “anti-CD20 antibody” or “an antibody that binds to CD20 or an epitope of CD20” refers to an antibody that is capable of binding CD20 with sufficient affinity such that the antibody is useful as a diagnostic and/or therapeutic agent in targeting CD20. The extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM.

CD20 is a hydrophobic transmembrane phosphoprotein that is expressed predominantly in pre-B cells and mature peripheral B cells in humans and mice. In humans, CD20 is also strongly and homogeneously expressed in most mature B-cell malignancies, including, for example, most non-Hodgkin's B-cell lymphomas (NHL) and B-type Chronic Lymphocytic Leukemia's (B-CLL). The CD20 antigen is not expressed on haematopoietic stem cells or on plasmocytes. Anti-CD20 monoclonal antibodies have been, and continue to be, developed for the treatment of B-cell diseases, including B-cell malignancies.

The chimeric anti-CD20 monoclonal antibody rituximab (Rituxan®) has become the standard therapy for many CD20-positive B-cell lymphomas and was the first mAb approved for any oncology indication. Demarest, S. J. et al., mAbs 3:338-351 (2011). Biosimilars of rituximab have now been FDA-approved, including rituximab-abbs (TRUXIMA) and rituximab-pvvr (RUXIENCE®). RITUXAN HYCELA® (rituximab and hyaluronidase human) injection for subcutaneous use was FDA-approved in 2017.

Aside from rituximab, a number of other anti-CD20 antibodies are also known in the art, including for example, ublituximab (TG-1101), ofatumumab (HuMax; Intracel), ocrelizumab, veltuzumab, GA101 (obinutuzumab), AME-133v (Applied Molecular Evolution), ocaratuzumab (Mentrik Biotech), PRO131921, tositumomab, ibritumomab-tiuxetan, hA20 (Immunomedics, Inc.), BLX-301 (Biolex Therapeutics), Reditux (Dr. Reddy's Laboratories), and PRO70769 (described in WO2004/056312).

Rituximab is a genetically engineered chimeric murine/human monoclonal antibody directed against the CD20 antigen. Rituximab is the antibody called “C2B8” in U.S. Pat. No. 5,736,137. The amino acid sequence of rituximab and exemplary methods for its production via recombinant expression in CHO cells are disclosed in U.S. Pat. No. 5,736,137, which is herein incorporated by reference in its entirety. Rituximab was initially approved by the FDA in 1997 for treating non-Hodgkin's lymphoma.

Ofatumumab is an anti-CD20 IgG1κ human monoclonal antibody. Studies indicated that ofatumumab dissociates from CD20 at a slower rate compared to rituximab and binds a membrane proximal epitope. Zhang et al., Mabs 1: 326-331 (2009). Epitope mapping has indicated that ofatumumab binds an epitope located closer to the N-terminus of CD20 compared to the location targeted by rituximab and includes an extracellular loop of the antigen. Id.

As used herein, “TG-1101” (TG Therapeutics, Inc.) (also known as ublituximab, UBX, UTX, TG-1101, TGTX-1101, Utuxin™, LFB-R603, TG20, EMAB603) is the source antibody for the anti-CD20 antibody described herein having a unique glycosylation profile that is produced by the currently disclosed methods.

The source antibody, TG-1101, is a monoclonal antibody that targets epitopes on CD20, e g., IRAHT (SEQ ID NO: 16), and EPAN (SEQ ID NO:17) See, Fox, E. et al., Mult. Scler. 27:420-429 (March 2021); Babiker et al., Expert Opin Investig Drugs 27:407-412 (2018); Cotchett, K R et al., Multiple Sclerosis and Related Disorders 49:102787 (2021); Miller et al., Blood 120: Abstract No. 2756 (2012): Deng, C. et. al., J. Clin. Oncol. 31:Abstract No. 8575 (2013). TG-1101 is also described in U.S. Pat. Nos. 9,234,045 and 9,873,745.

TG-1101 has been studied in a variety of patient cancer populations (e.g., NHL, CLL), both as a single agent and in combination with other agents. For example, O'Connor, O. A. et al., J. Clin. Oncol. 32:5s (2014), (suppl; Abstract No. 8524), showed that TG-1101 was well-tolerated and active in rituxin-exposed patients. In a phase I trial, Lunning, M. et al., American Society of Hematology Annual Meeting and Exposition, Dec. 5-8, 2015. Abstract No. 1538, showed that TG-1101 and TGR-1202 demonstrated activity and a favorable safety profile in relapsed/refractory B-cell NHL and high-risk CLL. And in a phase II trial, Sharman J. et. al., American Society of Hematology (ASH) Annual Meeting and Exposition. Dec. 5-8, 2015, Abstract No. 3980, showed that TG-1101 in combination with Ibrutinib was highly active in patients with relapsed and/or refractory mantle cell lymphoma. Results of several studies involving TG-1101 in combination with other agents (e.g., umbralisib, TG-1701, venetoclax) have been reported at the ASH Annual Meeting (Dec. 5-8, 2020), Publications 543, 3137, and 1130.

In addition, TG-1101 has been studied in phase 2 and 3 clinical trials to treat relapsing forms of multiple sclerosis (RMS). See, e.g., Fox, E. et al., Mult. Scler. 27:420-429 (March 2021) Steinman, L. et al., Neurology 96: (suppl 15) 4494 (2021). See, also, Pending U.S. Provisional Appl. Nos. 63/303,267, filed Jan. 26, 2022 and 63/288,350, filed Dec. 10, 2021, incorporated by reference in their entireties.

The amino acid (AA) and nucleotide sequences for antibody TG-1101 (TG Therapeutics, Inc.) are found in Table 11.

TABLE 11
Sequence Table for Source Anti-CD20 Antibody, TG-1101

| SEQ | | |
|---|---|---|
| ID | | |
| NO. | Description | Sequence |
| 1 | Variable | GYTFTSY |
| | heavy chain | |
| | (VH) CDR1 | |
| 2 | Variable | YPGNGD |
| | heavy chain | |
| | (VH) CDR2 | |
| 3 | Variable | YDYNYAMDY |
| | heavy chain | |
| | (VH) CDR3 | |
| 4 | Variable light | RASSSVSYMH |
| | chain (VL) | |
| | CDR1 | |
| 5 | Variable light | ATSNLAS |
| | chain (VL) | |
| | CDR2 | |
| 6 | Variable light | QQWTFNPPT |
| | chain (VL) | |
| | CDR3 | |
| 7 | Heavy chain | QAYLQQSGAE LVRPGASVKM SCKASGYTFT SYNMHWVKQT |
| | AA sequence | PRQGLEWIGG IYPGNGDTSY NQKFKGKATL TVGKSSSTAY |
| | | MQLSSLTSED SAVYFCARYD YNYAMDYWGQ GTSVTVSSAS |
| | | TKGPSVFPLA PSSKSTSGGT AALGCLVKDY FPEPVTVSWN |
| | | SGALTSGVHT FPAVLQSSGL YSLSSVVTVP SSSLGTQTYI |
| | | CNVNHKPSNT KVDKKVEPKS CDKTHTCPPC PAPELLGGPS |
| | | VFLFPPKPKD TLMISRTPEV TCVVVDVSHE DPEVKFNWYV |
| | | DGVEVHNAKT KPREEQYNST YRVVSVLTVL HQDWLNGKEY |
| | | KCKVSNKALP APIEKTISKA KGQPREPQVY TLPPSRDELT |
| | | KNQVSLTCLV KGFYPSDIAV EWESNGQPEN NYKTTPPVLD |
| | | SDGSFFLYSK LTVDKSRWQQ GNVFSCSVMH EALHNHYTQK |
| | | SLSLSPGK |
| 8 | Light chain | QIVLSQSPAI LSASPGEKVT MTCRASSSVS YMHWYQQKPG |
| | AA sequence | SSPKPWIYAT SNLASGVPAR FSGSGSGTSY SFTISRVEAE |
| | | DAATYYCQQW TFNPPTFGGG TRLEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| 9 | Light chain | QIVLSQSPAI LSASPGEKVT MTCRASSSVS YMHWYQQKPG |
| | with one AA | SSPKPWIYAT SNLASGVPAR FSGSGSGTSY SFTISRVEAE |
| | mismatch at | DAATYYCQQW TFNPPTFGGG TRLEINRTVA APSVFIFPPS |
| | position 106 | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| 10 | Variable | QAYLQQSGAE LVRPGASVKM SCKASGYTFT SYNMHWVKQT |
| | heavy chain | PRQGLEWIGG IYPGNGDTSY NQKFKGKATL TVGKSSSTAY |
| | (VH) | MQLSSLTSED SAVYFCARYD YNYAMDYWGQ GTSVTVSS |
| 11 | Constant | ASTKGPSVFP LAPSSKSTSG GTAALGCLVK DYFPEPVTVS |
| | heavy chain | WNSGALTSGV HTFPAVLQSS GLYSLSSVVT VPSSSLGTQT |
| | | YICNVNHKPS NTKVDKKVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSRDE |
| | | LTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 12 | Variable light | QIVLSQSPAI LSASPGEKVT MTCRASSSVS YMHWYQQKPG |
| | chain (VL) | SSPKPWIYAT SNLASGVPAR FSGSGSGTSY SFTISRVEAE |
| | | DAATYYCQQW TFNPPTFGGG TRLEIKR |
| 13 | Constant | TVAAPSVFIF PPSDEQLKSG TASVVCLLNN FYPREAKVQW |
| | light chain | KVDNALQSGN SQESVTEQDS KDSTYSLSST LTLSKADYEK |
| | | HKVYACEVTH QGLSSPVTKS FNRGEC |
| 14 | Nucleic acid | caggcttatctacagcagtctggggctgagctggtgaggcctggggcctcagtgaagatgtcctgcaaggct |
| | sequence of | tctggctacacatttaccagttacaatatgcactgggtaaagcagacacctagacagggcctggaatggatt |
| | heavy chain | ggaggtatttatccaggaaatggtgatacttcctacaatcagaagttcaagggcaaggccacactgactgta |
| | (HC) | ggcaaatcctccagcacagcctacatgcagctcagcagcctgacatctgaagactctgcggtctatttctgt |
| | | gcaagatatgactacaactatgctatggactactggggtcaaggaacctcagtcaccgtctcctcagcctcc |
| | | accaagggcccatcggtcttccccctggcaccctcctccaagagcacctctgggggcacagcggccctgggc |
| | | tgcctggtcaaggactacttccccgaaccggtgacggtgtcgtggaactcaggcgccctgaccagcggcgtg |
| | | cacaccttcccggctgtcctacagtcctcaggactctactccctcagcagcgtggtgaccgtgccctccagc |
| | | agcttgggcacccagacctacatctgcaacgtgaatcacaagcccagcaacaccaaggtggacaagaaagtt |
| | | gagcccaaatcttgtgacaaaactcacacatgcccaccgtgcccagcacctgaactcctggggggaccgtca |
| | | gtcttcctcttccccccaaaacccaaggacaccctcatgatctcccggacccctgaggtcacatgcgtggtg |
| | | gtggacgtgagccacgaagaccctgaggtcaagttcaactggtacgtggacggcgtggaggtgcataatgcc |
| | | aagacaaagccgcgggaggagcagtacaacagcacgtaccgtgtggtcagcgtcctcaccgtcctgcaccag |
| | | gactggctgaatggcaaggagtacaagtgcaaggtctccaacaaagccctcccagcccccatcgagaaaacc |
| | | atctccaaagccaaagggcagccccgagaaccacaggtgtacaccctgcccccatcccgggatgagctgacc |
| | | aagaaccaggtcagcctgacctgcctggtcaaaggcttctatcccagcgacatcgccgtggagtgggagagc |
| | | aatgggcagccggagaacaactacaagaccacgcctcccgtgctggactccgacggctccttcttcctctac |
| | | agcaagctcaccgtggacaagagcaggtggcagcaggggaacgtcttctcatgctccgtgatgcatgaggct |
| | | actgcacaaccctacacgcagaagagcctctccctgtctccgggtaaa |
| 15 | Nucleic acid | caaattgttctctcccagtctccagcaatcctgtctgcatctccaggggagaaggtcacaatgacttgcagg |
| | sequence of | gccagctcaagtgtaagttacatgcactggtaccagcagaagccaggatcctcccccaaaccctggatttat |
| | light chain | gccacatccaacctggcttctggagtccctgctcgcttcagtggcagtgggtctgggacctcttattctttc |
| | (LC) | acaatcagcagagtggaggctgaagatgctgccacttattactgccagcagtggacttttaacccacccacg |
| | | ttcggaggggggaccaggctggaaataaaacggactgtggctgcaccaagtgtcttcatcttcccgccatct |
| | | gatgagcagttgaaatctggaactgcctctgttgtgtgcctgctgaataacttctatcccagagaggccaaa |
| | | gtacagtggaaggtggataacgccctccaatcgggtaactcccaggagagtgtcacagagcaggacagcaag |
| | | gacagcacctacagcctcagcagcaccctgacgctgagcaaagcagactacgagaaacacaaagtctacgcc |
| | | tgcgaagtcacccatcagggcctgagctcgcccgtcacaaagagcttcaacaggggagagtgt |
| 16 | Epitope on | IRAHT |
| | CD20 | |
| 17 | Epitope on | EPAN |
| | CD20 | |

In some aspects, TG-1101 comprises the VH CDR1, CDR2, and CDR3 region of sequences SEQ ID NOS: 1, 2, and 3, and the VL CDR1, CDR2, and CDR3 region of sequences SEQ ID NOS: 4, 5, and 6.

In some aspects, TG-1101 comprises a heavy chain (HC) having at least 95% identity to an amino acid sequence set forth in SEQ ID NO:7 and a light chain (LC) having at least 95% identity to an amino acid sequence set forth in SEQ ID NO:8.

In some aspects, TG-1101 comprises the HC of SEQ ID NO: 7 and the LC of SEQ ID NO: 8.

In some aspects, TG-1101 comprises the HC of SEQ ID NO: 7 and the LC of SEQ ID NO: 9.

In some aspects, the monoclonal antibody produced by the methods disclosed herein comprises: a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some aspects, the monoclonal antibody produced by the methods disclosed herein comprises a heavy chain having at least 90%, 91%, 92%, 93%, 94%, 95%, 96%, 97%, 98%, or at least 99% identity to an amino acid sequence set forth in SEQ ID NO:7; and a light chain having at least 90%, 91%, 92%, 93%, 94%, 95%, 96%, 97%, 98%, or at least 99% identity to an amino acid sequence set forth in SEQ ID NO: 8.

In some aspects, the monoclonal antibody produced by the methods disclosed herein comprises a heavy chain having at least 95% identity to an amino acid sequence set forth in SEQ ID NO:7 and a light chain having at least 95% identity to an amino acid sequence set forth in SEQ ID NO: 8.

In some aspects, the monoclonal antibody produced by the methods disclosed herein comprises a heavy chain having an amino acid sequence set forth in SEQ ID NO:7 and a light chain having an amino acid sequence set forth in SEQ ID NO:8.

In some aspects, the monoclonal antibody produced by the methods disclosed herein comprises a heavy chain having an amino acid sequence set forth in SEQ ID NO:7 and a light chain having an amino acid sequence set forth in SEQ ID NO:9.

In some aspects, the monoclonal antibody produced by the methods disclosed herein comprises a deletion of up to 5 N-terminal residues.

In some aspects, the monoclonal antibody produced by the methods disclosed herein comprises a deletion of up to 10 N-terminal sequences.

In some aspects, the monoclonal antibody produced by the methods disclosed herein comprises the VH of SEQ ID NO:10 and the VL of SEQ ID NO: 12.

In some aspects, the monoclonal antibody produced by the methods disclosed herein bind to the same epitope as TG-1101 (TG Therapeutics, Inc.). In some aspects, the monoclonal antibodies produced by the methods disclosed herein are anti-CD20 antibodies (i.e., binds an epitope of CD20).

Transfection of an Expression Vector into Rat Hybridoma Cells

A nucleic acid sufficient to achieve expression (typically an expression vector containing the gene encoding the polypeptide or protein of interest and any operably linked genetic control elements) are introduced into the rat hybridoma cells by any number of well-known techniques. The term “transfection,” as used herein, refers to the introduction of one or more exogenous polynucleotides (e.g., antibodies) into a rat hybridoma cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature 346: 776-777 (1990)); and strontium phosphate DNA co-precipitation (Brash et al., Mol. Cell Biol. 7: 2031-2034 (1987)).

For example, as described in Example 3, the expression vector HK463-25, (see FIG. 20) containing the immunoglobulin heavy and light chain cDNA sequences of TG-1101 (TG Therapeutics, Inc.), was transfected into YB2/0 host cells to produce an anti-CD20 antibody, TG-1101 in a 15,000 L production bioreactor.

Cells are screened to determine which of the rat hybridoma cells have actually taken up the vector and express the polypeptide or protein of interest. Traditional methods of detecting a particular polypeptide or protein of interest expressed by mammalian cells include, but are not limited to, immunohistochemistry, immunoprecipitation, flow cytometry, immunofluorescence microscopy, SDS-PAGE, Western blots, enzyme-linked immunosorbent assay (ELISA), high performance liquid chromatography (HPLC) techniques, biological activity assays, and affinity chromatography.

### Rat Hybridoma Cell Culture

Once a cell that expresses the polypeptide or protein of interest has been identified, the cell is propagated in culture by any of the variety of methods well-known to those skilled n the art. The cell expressing the protein of interest is typically propagated by growing it at a temperature and in a medium that is conducive to the survival, growth and viability of the cell. The initial culture volume can be of any size, but is often smaller than the culture volume of the production bioreactor used in the final production of the protein of interest, and frequently cells are passaged several times in bioreactors of increasing volume prior to seeding the production bioreactor. The cell culture can be agitated or shaken to increase oxygenation of the medium and dispersion of nutrients to the cells. Alternatively or additionally, special sparging devices that are well known in the art can be used to increase and control oxygenation of the cell culture. In accordance with the present disclosure, one of ordinary skill in the art will understand that it can be beneficial to control or regulate certain internal conditions of the bioreactor, including but not limited to pH temperature, oxygenation, etc.

The starting cell density in the production bioreactor can be chosen by one of ordinary skill in the art. In accordance with the present disclosure, the starting cell density in the production bioreactor can be as low as a single cell per culture volume. In some aspects, starting cell densities in the production bioreactor can range from about 0.1×10⁶ to about 10×10⁶ viable cells per mL. In some aspects, starting cell densities in the production bioreactor can range from about 0.1×10⁶ to about 2.0×10⁶. In some aspects, starting cell densities in the production bioreactor can be 2×10², 2×10³, 2×10⁴, 2×10⁵, 2×10⁶, 5×10⁶, or 10×10⁶ viable cells per mL, and higher.

Initial and intermediate cell cultures may be grown to any desired density before seeding the next intermediate or final production bioreactor. It is preferred that most of the cells remain alive prior to seeding, although total or near total viability is not required. In one embodiment of the present disclosure, the cells may be removed from the supernatant, for example, by low-speed centrifugation. It may also be desirable to wash the removed cells with a medium before seeding the next bioreactor to remove any unwanted metabolic waste products or medium components. The medium may be the medium in which the cells were previously grown or it may be a different medium or a washing solution selected by the practitioner of the present disclosure.

The cells may then be diluted to an appropriate density for seeding the production bioreactor. In a preferred aspect of the present disclosure, the cells are diluted into the same medium that will be used in the production bioreactor. Alternatively, the cells can be diluted into another medium or solution, depending on the needs and desires of the practitioner of the present disclosure or to accommodate particular requirements of the cells themselves, for example, if they are to be stored for a short period of time prior to seeding the production bioreactor.

Once the production bioreactor has been seeded, as described above, the cell culture enters an “initial growth phase.” In certain aspects, the “initial growth phase” refers to culture days 0 to 2 (i.e., days 0, 1, and/or 2) of the cell culture during which the cells (e.g., YB2/0) begin to grow. In the “initial growth phase,” the amount of recombinant protein expressed by the cell culture is significantly lower than in the “protein generation phase” of the cell culture (i.e., culture days 3 to harvest). The precise conditions will vary depending on the cell type, the organism from which the cell was derived, and the nature and character of the expressed recombinant protein.

Following the “initial growth phase” is the “protein generation phase” of the cell culture. In certain aspects, the “protein generation phase” refers to culture days 3 to harvest, during which the cells (e.g., YB2/0) produce recombinant protein at significantly higher amounts than in the initial growth phase, such that protein titers can be measured. The precise conditions will vary depending on the cell type, the organism from which the cell was derived, and the nature and character of the expressed recombinant protein.

In accordance with the present disclosure, the production bioreactor can be any volume that is appropriate for large-scale production of recombinant proteins. In one aspect, the volume of the production bioreactor is at least 500 liters. In other aspects, the volume of the production bioreactor is 1000, 2500, 5000, 8000, 10,000, 12,000, 15,000, 20,000, 25,000 liters or more, or any volume in between. One of ordinary skill in the art will be aware of and will be able to choose a suitable bioreactor for use in practicing the present disclosure. The production bioreactor may be constructed of any material that is conducive to cell growth and viability that does not interfere with expression or stability of the produced polypeptide or protein.

In some aspect, the cells are grown during the initial growth phase for a period of time sufficient to achieve a viable cell density that is a given percentage of the maximal viable cell density that the cells would eventually reach if allowed to grow undisturbed. For example, the cells may be grown for a period of time sufficient to achieve a desired viable cell density of 1, 5, 10, 15, 20, 25, 30, 35, 40, 45, 50, 55, 60, 65, 70, 75, 80, 85, 90, 95 or 99 percent of maximal viable cell density.

In some aspects, the cells are allowed to grow for a defined period of time. For example, depending on the starting concentration of the cell culture, the temperature at which the cells are grown, and the intrinsic growth rate of the cells, the cells may be grown in culture for 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days. In some cases, the cells may be allowed to grow for a month or more. The cells would be grown for 0 days in the production bioreactor if their growth in a seed bioreactor, at the initial growth phase temperature, was sufficient that the viable cell density in the production bioreactor at the time of its inoculation is already at the desired percentage of the maximal viable cell density.

The cell culture may be agitated or shaken during the initial growth phase in order to increase oxygenation and dispersion of nutrients to the cells. In accordance with the present disclosure, certain internal conditions of the bioreactor may be controlled or regulated during the initial growth phase, including but not limited to pH temperature, oxygenation, etc. For example, pH can be controlled by supplying an appropriate amount of acid or base and oxygenation can be controlled with sparging devices that are well known in the art.

Various methods of culturing mammalian cells for production of recombinant proteins by batch, fed-batch, continuous, semi-continuous, and perfusion culture modes are known in the art. See, e.g., Willard, S. S., M., BioProcess Int. 15(3) 38-46 (2017), discussing culture methods in monoclonal antibody production.

In a preferred aspect, the rat hybridoma cells, as described herein, are cultured using a fed-batch operation mode. As used herein, the terms “fed-batch” or “fed-batch culture” refer to a method of culturing cells in which additional components are provided to the culture at some time after the beginning of the culture process. In some aspects of the fed-batch method, nutrients are added once they are depleted. A fed-batch culture can be started using a basal culture medium (e.g., CDM4Mab®). The culture medium with which additional components are provided to the culture at some time subsequent to the beginning of the culture process is a feed medium (e.g., BalanCD CHO Feed 4®). A fed-batch culture is typically stopped at some point (based on number of culture days or cell viability, whichever comes first) and the cells and/or components in the medium are harvested and purified.

Alternative culture modes, such as batch and perfusion operation modes are also envisioned for use with the methods of the disclosure, however, those skilled in the art would understand that some modifications may need to be made using routine experimentation. For example, the skilled artisan would understand that in batch mode, there would be no use of feed media. As used herein, “batch culture” or “batch operation mode” refers to a cell culture mode where cells are grown in a fixed volume of nutrient culture media under specific environmental conditions up to a certain density, and then harvested and processed as a batch before the nutrients are used up. As used herein, “perfusion culture” or “perfusion operation mode” refers to a cell culture mode having a continuous flow of a physiological nutrient solution at a steady rate, through or over a population of cells.

### Monitoring Cell Culture Conditions

In some aspects of the presently disclosed methods, particular conditions of the growing cell culture are periodically monitored. Monitoring cell culture conditions allows the practitioner to determine whether the cell culture is producing recombinant protein at suboptimal levels or whether the culture is about to enter into a suboptimal production phase. In order to monitor certain cell culture conditions, it will be necessary to remove small aliquots of the culture for analysis.

As a non-limiting example, it may be beneficial or necessary to monitor temperature, pH, cell density, viable cell density, cell viability, integrated viable cell density, lactate levels, ammonium levels, osmolality, amount of dissolved oxygen, pCO₂ levels, glutamine levels, glutamate levels, or glucose levels of the cell culture, or the titer of the expressed polypeptide or protein. In some aspects, such parameters are measured on a periodic basis. In some aspects, such parameters are measured one or more times a day (i.e., 1, 2, 3, 4, 5). In some aspects, such parameters are measured on a daily basis. In some aspects, such parameters are measured every other day. In some aspects, such measurements are taken during the protein generation phase of the cell culture. In some aspects, such measurements are taken during the initial growth phase of the cell culture. In some embodiments, the expression or activity level of the expressed recombinant protein is measured on day 1, 2, 3, 4, 5, 6, 7, 8, 9, 10, 11, 12, 13, 14, 15, 16, 17, 18, 19, 20, or 21 following the start of cell culture.

Numerous techniques are well known in the art that will allow one of ordinary skill in the art to measure these conditions. For example, cell density may be measured using a hemacytometer, a Coulter counter, or cell density examination (CEDEX). Viable cell density may be determined by staining a culture sample with Trypan blue. Since only dead cells take up the Trypan blue, viable cell density can be determined by counting the total number of cells, dividing the number of cells that take up the dye by the total number of cells, and taking the reciprocal. Cell viability can also be measured using a biomass capacitance probe. HPLC can be used to determine the levels of lactate, ammonium or the expressed polypeptide or protein. Alternatively, the level of the expressed polypeptide or protein can be determined by standard molecular biology techniques such as coomassie staining of SDS-PAGE gels, Western blotting, Bradford assays, Lowry assays, Biuret assays, and UV absorbance. It may also be beneficial or necessary to monitor the post-translational modifications of the expressed polypeptide or protein, including phosphorylation and glycosylation.

### Shifting Process Parameters in Rat Hybridoma (e.g., YB2/0) Cell Culture

In some aspects of the present disclosure, rat hybridoma cells are cultured under culture conditions that promote and optimize production, titer, and product quality of the recombinant protein (e.g., monoclonal antibodies) being expressed. For example, in some aspects, the cell culture may be shifted by shifting one or more of a number of culture conditions including, for example, temperature, pH, osmolality, and sodium butyrate levels.

In some aspects, the following process parameters can be shifted during the protein generation phase of the cell culture (culture days 3→harvest). In certain aspects, process parameters can be shifted during the initial growth phase (culture days 0→2) of the cell culture.

In some aspects, the process parameter shifts described below do not occur at the same time. In some aspects, the process parameter shifts described below may occur at the same time or within at least 1, at least 2, at least 3, at least 4, at least 5, at least 6, at least 7, at least 8, at least 9, at least 10 hours of each other. In some aspects, one particular process parameter shift (e.g., temperature) does not routinely precede or proceed another process parameter shift (e.g., pH). The process parameter shifts, as disclosed herein, can occur alone or can occur in combination.

Controlling pH and Levels of pCO₂

Both pCO₂ and culture pH impact product quality profiles of recombinant glycoproteins. Brunner, M. et al., Bioprocess and Biosystems Engineering 40: 251-263 (2017). pH control strategies are principally important towards controlling pCO₂ levels. pH is an important process control parameter. Culture pH is principally important for mammalian cell physiology. Typical pH control ranges for mammalian cell lines in culture are 6.7-7.3. To help support pH maintenance in cell culture media, various buffers are supplemented into cell culture media. Sodium bicarbonate is one of the most common. Alternative buffering systems, including HEPES, are also typically used. See, Itagaki, A. et al., Experimental Cell Research 83:351-361 (1974). For sodium bicarbonate buffers, control of culture pH is typically two-sided. When pH exceeds a desired value, carbon dioxide is added into the bioreactor to decrease pH to an acceptable value. When pH drops below a desired value, a dilute basic solution is typically added into the bioreactor to increase pH to an acceptable value. Alternative strategies for pH control in mammalian cell bioreactors have been interrogated. The choice of pH control methodology and buffering system is ultimately up to the manufacturer of a therapeutic protein. Excessive base and CO₂ addition and accumulation in bioreactors have been shown to be detrimental to growth for mammalian cells in culture. See, deZengotita, V. et al., Cytotechnology 28:213-227 (1998). Thus, controlling the levels of dissolved CO₂ (pCO₂) and base addition are important for overall culture process performance. Cell culture process variables such as pH and pCO₂ are thus closely monitored during manufacturing. So too is the product quality of the expressed protein after harvesting and purifying from cell culture.

pH

In some aspects, the method of making a recombinant protein in rat hybridoma cells comprises culturing the rat hybridoma cells in a cell culture having a culture pH of about 6.5 to about 7.55, wherein the rat hybridoma cells comprise an expression vector comprising a polynucleotide encoding the recombinant protein. In some aspects, the culture pH is about 6.5 to about 7.0. In some aspects, the culture pH of about 6.5 to about 7.0 is set on culture day 2 of the cell culture. In some aspects, the culture pH of about 6.5 to about 7.0 is set on culture day 3 of the cell culture. In some aspects, the culture pH is about 7.0 to about 7.55. In some aspects, the culture pH of about 7.0 to about 7.55 is set on culture days 0 to 3 of the cell culture.

In some aspects, the culture pH is lowered (or shifted) on day 2 or day 3 of the cell culture to about 6.5 to about 7.0. In some aspects, the culture pH is lowered on day 3 of the cell culture. In some aspects, the culture pH of about 6.5 to about 7.0 is maintained from culture day 3 until harvest of the cell culture. In some aspects, the timing of the day 3 pH shift occurs in the production bioreactor 62-77 hours post-inoculation.

In some aspects, the recombinant protein's harvest titer is increased and/or fucosylation of the recombinant protein is decreased when the culture pH is 6.6 to 6.96 relative to a cell culture under the same culture conditions except that the culture pH is 6.60 to 6.8

Another pH-related process parameter that has been monitored in the rat hybridoma cell culture disclosed herein is called the “integrated pH2 difference.” See, Example 5. Integrated pH2 difference refers to the cumulative culture time that pH is allowed to drop below a fixed pH setpoint, and the cumulative magnitude of that drop, after the pH is lowered on culture day 3. The integrated pH2 difference increases when culture pH drops below a fixed pH setpoint for any unit measure of time. The fixed pH setpoint is an assigned value. In some aspects, the fixed pH setpoint is pH 6.91. In some aspects, the integrated pH2 difference of a cell culture relative to a fixed pH setpoint (e.g., pH 6.91) is less, relative to a cell culture with an integrated pH2 difference that is more. In some aspects, lower integrated pH2 difference results in higher integrated viable cell density (IVCD) and higher titer at harvest. In some aspects, lower integrated pH2 difference further results in lower percent fucosylation.

pCO₂

In cell culture media with sodium bicarbonate-based buffer, CO₂ is introduced into the cell culture to control pH. When pH increases above a targeted value/range, CO₂ is added to reduce the pH. The exogenously added CO₂ is dissolved into the culture media increasing pCO₂ levels. In some aspects, by virtue of the control of the culture pH, as described herein, the culture conditions further comprise controlling culture pCO₂ levels to less than about 300 mmHg. As used herein, “less than about 300 mmHg” means that the pCO₂ level can range from 0 to 300 mmHg.

There are multiple cell culture process means, other than higher pH setpoints, to support higher pH control levels while simultaneously controlling low pCO₂ levels. An alternative means to facilitate low pCO₂ levels includes increasing the buffering capacity of the cell culture media by supplementing with additional buffers such as HEPES. By reducing the need for CO₂ addition into the bioreactor to control pH, this would attenuate culture-related increases in pCO₂ during the culture. Another alternative means to facilitate low pCO₂ levels in the bioreactor is to increase the air overlay and air sparge rates so as to increase the pCO₂ stripping capability of the bioreactor and prevent high levels from being realized. Increasing the dissolved oxygen (DO) setpoint, and/or decreasing the bioreactor agitation rate would have a similar effect of forcing higher air and O₂ flowrates into the bioreactor to maintain DO, effectively flushing excess pCO₂ out of the bioreactor.

In some aspects of the presently disclosed methods, the pCO₂ levels of less than about 300 mmHg are facilitated by supplementing the cell culture with additional buffers, increasing the air sparge rate, increasing the DO setpoint, and/or decreasing the agitation rate.

In some aspects, the cell culture is conducted in a commercial-scale bioreactor. In some aspects, the commercial-scale bioreactor is a 10,000 L, 15,000 L, 20,000 L, or 25,000 L bioreactor. In some aspects, the commercial scale bioreactor is a 15,000 L bioreactor.

In some aspects, the rat hybridoma cells are YB2/0 rat hybridoma cells.

In some aspects, the recombinant protein is an IgG1 glycoprotein.

In some aspects, the recombinant protein is a monoclonal antibody. In some aspects, the monoclonal antibody is a chimeric, humanized, or human antibody.

In some aspects, the monoclonal antibody specifically binds an epitope of CD20 (i.e., is an anti-CD20 antibody).

In some aspects, the monoclonal antibody is TG-1101. In some aspects, the monoclonal antibody binds the same epitope as TG-1101.

In some aspects, additional cell culture process parameters, such as, e.g., temperature control, as described below, are shifted in conjunction with the shift in culture pH or the surrogate shift in pCO₂ in order to optimally express a recombinant protein of interest (e.g., monoclonal antibody) in rat hybridoma cells.

### Temperature Shift

In the presently disclosed methods, temperature shifts of the cell culture are another culture condition for increasing expression, cell density or viability, and product quality of a recombinant protein in a rat hybridoma cell line. In some aspects, it may also be desirable to use multiple discrete temperature shifts at different times during the cell culture.

In some aspects, temperature shift of the cell culture in rat hybridoma cells is carried out in combination with a pH shift, as described herein. In certain aspects, temperature shift of the cell culture is carried out in combination with a pH shift and in combination with the use of chemically-defined and ADCF cell culture media. In some aspects, the chemically-defined and ADCF cell culture media is CDM4Mab®. In some aspects, feed media BalanCD CHO Feed 4® is used together with CDM4Mab®.

In some aspects, the culture conditions comprise an initial temperature set point of about 37° C., wherein said initial temperature set point is set on culture day 0 to culture day 1.

In some aspects, the culture conditions further comprise a second temperature set point of about 35° C., wherein said second temperature set point is set at the end of culture day 1 to culture day 3. As used herein, the “end of culture day 1” refers to 17 to 33 hours after the start of the cell culture.

In some aspects, the culture conditions further comprise a third temperature set point of about 32° C. to about 33° C., wherein said third temperature set point is set on culture day 3 and is maintained though harvest. In some aspects, the third temperature set point is 32.5° C.

In some aspects, the cell culture comprises the following culture conditions: i). an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set culture day 3 and is maintained though harvest; ii). a culture pH between about 6.5 to about 7.55; and iii). a culture pCO₂ less than about 300 mmHg.

In some aspects, “culture day 1” refers to about 12.0 hours to about 35.9 hours post-inoculation of the cells in the seed culture to the production bioreactor. In some aspects, “culture day 2” refers to about 36.0 hours to about 59.9 hours post-inoculation of the cells in the seed culture to the production bioreactor. In some aspects, “culture day 3” refers to about 60.0 hours to about 83.9 hours post-inoculation of the cells in the seed culture to the production bioreactor.

In some aspects, the timing of the first temperature shift occurs in the production bioreactor 17-33 hours post-inoculation. In some aspects, the timing of the second temperature shift occurs in the production bioreactor 62-77 hours post-inoculation.

In some aspects, the recombinant protein harvest titer in rat hybridoma cells is about 0.5 g/L to about 1.5 g/L when culture pH is controlled and temperature shifted, as described herein. In some aspects, the recombinant protein harvest titer in rat hybridoma cells is about 0.5 g/L to about 1.3 g/L. In some aspects, the recombinant protein harvest titer in rat hybridoma cells is about 1.0 g/L to about 1.5 g/L.

In some aspects, yield of the recombinant protein is increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to a recombinant protein produced by a culturing process that does not employ i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set culture day 3 and is maintained though harvest; ii) a culture pH between about 6.5 to about 7.55; and iii) a culture pCO₂ less than about 300 mmHg.

In some aspects, the percent fucosylation of the recombinant protein prepared when culture pH is controlled and temperature shifted, as described herein, is between about 20% and about 35%. In some aspects, the percent fucosylation of the recombinant protein prepared when culture pH is controlled and temperature shifted, as described herein, is about 20% to about 30%.

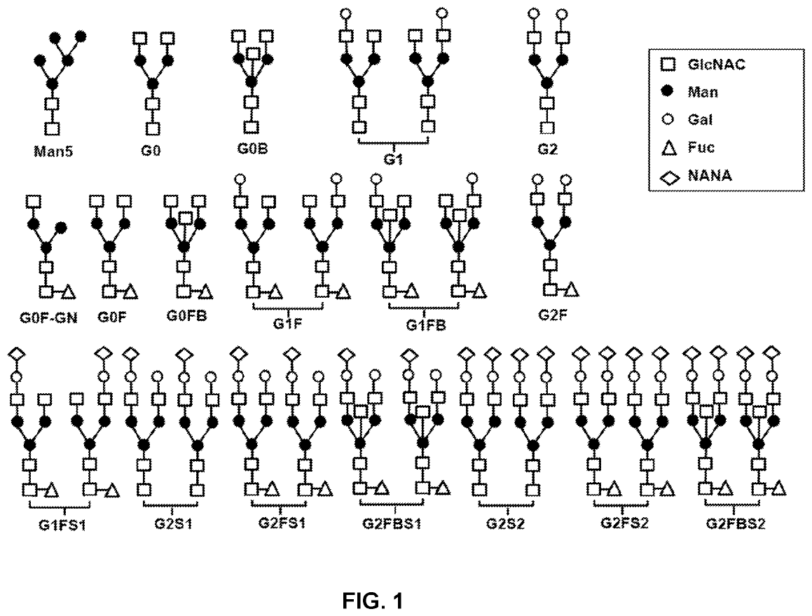

In some aspects, the recombinant proteins or anti-CD20 antibodies made by the methods disclosed herein comprise between 20% and 40% fucosylated glycans; between 23% and 36% fucosylated glycans; between 28% and 33% fucosylated glycans; or about 33% or about 36% fucosylated glycans (wherein “about” means+/−1, 2, 3, 4, 5, 6, 7, 8, 9, or 10%). Fucosylated glycans are those N-glycans shown in FIG. 1 that carry a fucose residue (shown as open triangle in FIG. 1). In short, a sample or a population of anti-CD20 antibodies is subjected to enzymatic deglycosylation so that all N-glycans are cleaved from the core. The resulting N-glycans can subsequently be analyzed, e.g., by mass spectrometry. The percent of fucosylated N-glycans is the percent of fucosylated N-glycans among N-glycans that were cleaved using the enzymatic digest.

In certain aspects, the recombinant proteins or anti-CD20 antibodies made by the methods disclosed herein comprise between at least 20%, 21%, 22%, 23%, 24%, 25%, 26%, 27%, 28%, 29%, 30%, 31%, 32%, 33%, 34%, 35%, 36%, 37%, 38%, 39%, or at least 40%, and at most 22%, 23%, 24%, 25%, 26%, 27%, 28%, 29%, 30%, 31%, 32%, 33%, 34%, 35%, 36%, 37%, 38%, 39%, or at most, 40% fucosylated glycans. In some aspects, the recombinant proteins or anti-CD20 antibodies comprise about 36% fucosylated glycans.

In some aspects, sialylation of the recombinant protein or anti-CD20 antibodies made by the methods disclosed herein is about 1% to about 4% when culture pH is controlled and temperature shifted, as described herein.

In some aspects, the recombinant protein or anti-CD20 antibodies made by the methods disclosed herein comprise less than 10%, 8%, 5%, 4%, 3%, 2.5%, 2%, 1%, or 0.5% sialylated glycans. In some aspects, the recombinant protein or anti-CD20 antibodies made by the methods disclosed herein comprises between 10% and 0.5% sialylated glycans; between 10% and 5% sialylated glycans; between 5% and 0.5% sialylated glycans; between 4% and 0.5% sialylated glycans; between 2% and 0.5% sialylated glycans; or no detectable amount of sialylated glycans. In short, a sample or a population of anti-CD20 antibodies are subjected to enzymatic deglycosylation so that all N-glycans are cleaved from the core. The resulting N-glycans can subsequently be analyzed, e.g., by mass spectrometry. The percent of sialylated N-glycans is the percent of sialylated N-glycans among N-glycans that were cleaved using the enzymatic digest.

In certain aspects, the recombinant protein or anti-CD20 antibodies made by the methods disclosed herein comprises between at least no detectable amount, 0.5%, 1%, 2%, 3%, 4%, or at least 5% and at most 0.5%, 1%, 2%, 3%, 4%, 5% or at most 10% sialylated glycans. In some aspects, the recombinant protein or anti-CD20 antibodies comprises no detectable amount of sialylated glycans.

In some aspects, the culture conditions of temperature shift and pH control as described herein further comprise culturing the cells in cell culture basal media that is chemically defined and animal-derived component free (ADCF), such as, e.g., CDM4Mab® (Cytiva Lifesciences/GE) and feed media BalanCD CHO Feed 4® (Irvine Scientific). In some aspects, the feed media is added to the cell culture about every 48 hours. In some aspects, glucose, glutamine, and/or a cholesterol lipid solution are added to supplement the feed media in the cell culture.

In some aspects, the method of making a recombinant protein in rat hybridoma cells utilizing the cell culture pH control and temperature control, as described herein, and culturing the cells in chemically defined and animal-derived component free (ADCF) cell culture media, e.g., CDM4Mab® and feed media BalanCD CHO Feed 4®, results in a recombinant protein harvest titer of about 0.5 g/L to about 1.3 g/L. In some aspects, the cell culture is conducted in a bioreactor. In some aspects, the cell culture is conducted in a commercial-scale bioreactor (e.g., 10,000 L to 25,000 L). In some aspects, the cell culture is conducted in a 15,000 L bioreactor.

### Cell Culture Media and Feed Media

Hundreds of basal cell culture media formulations for use in mammalian cell culture are well-known in the art and commercially available, including serum-free, peptone free, animal-derived component-free (ADCF), and/or chemically defined culture media. To these basal culture medium formulations, the skilled artisan will add components such as, e.g., amino acids, salts, sugars, vitamins, hormones, growth factors, buffers, antibiotics, lipids, trace elements and the like, depending on the requirements of the particular type of host cells to be cultured.

Cell culture media typically include at least one or more components from the following: an energy source (e.g., in the form of a carbohydrate such as glucose); essential amino acids, including the twenty basic amino acids plus cysteine; vitamins and/or other organic compounds typically required at low concentrations; lipids or free fatty acids (e.g., linoleic acid); and trace elements (e.g., inorganic compounds or naturally occurring elements that are typically required at very low concentrations, usually in the micromolar range). Media may be solid, gelatinous, liquid, gaseous or a mixture of phases and materials.

Some examples of commercially available cell culture basal media are listed in Example 1, Table 2, and include, e.g., Hycell Medium, IS CHO-SD G10.6 Medium, CD Hybridoma, PFHM-II Protein Free Hybridoma, Ex-Cell CD Hybridoma, UltraDOMA-PF Hybridoma, ProDOMA 1, ProDOMA 1, liquid media (LM)| CDM4Mab, and ActiCHO-P.

Additionally, the cell culture media can optionally be supplemented to include one or more additional components, in appropriate concentrations or amounts, as necessary or desired, and as would be known and practiced by those of ordinary skill in the art. Supplements that support the growth and maintenance of particular cell cultures are able to be readily determined by those of ordinary skill in the art, such as is described, for example, in Barnes et al., Cell 22:649 (1980); in Mammalian Cell Culture, Mather, J. P., ed., Plenum Press, NY (1984); and in U.S. Pat. No. 5,721,121.

Exemplary supplements include, but are not limited to, chemical gene selection agents, hormones and other growth factors, (e.g., insulin, transferrin, epidermal growth factor, serum, somatotropin, pituitary extract, aprotinin); salts (e.g., calcium, magnesium and phosphate), and buffers (e.g., HEPES (4-[2-Hydroxethyl]-1-piperazine-ethanesulfonic acid)); nucleosides and bases (e.g., adenosine, thymidine, hypoxanthine); protein and hydrolysates; antibiotics (e.g., gentamycin); cell protective agents (e.g., a Pluronic polyol (PLURONIC® F68)) and extracellular matrix proteins (e.g., fibronectin).

In some aspects, the culture media suitable for the presently disclosed methods is supplemented with a feed medium. In some aspects, the feed medium is a chemically defined feed medium. In some aspects, chemically defined feed media (or CDFM) or medium refers to media which contain one or more nutrients whose chemical composition and relative concentrations are known, and which is added to the culture media beginning at some time after inoculation. CDFM is supplied to the culturing vessel continuously or in discrete increments, to the culture media during culturing, with or without periodic cell and/or product harvest before termination of the cell culture. CDFM can be individually formulated to comprise a unique blend of amino acids, vitamins, trace minerals, and organic compounds, at enriched amounts to serve as a feed media to cell culture media. Alternatively, commercially available CDFM can be used. Some examples of commercially available CDFM are listed in Example 1, Table 3, and include EX-CELL Advanced CHO Feed 1 (with Glucose); Cell Boost 6; CHO CD Efficient Feed A; BalanCD CHO feed 4; Cell Boost 7A and b; and Cell Boost 3.

In some aspects, basal culture media CDM4Mab® (Cytiva Lifesciences/GE) is used in combination with feed media BalanCD CHO Feed 4® (Irvine Scientific) in the rat hybridoma cell culture.

As disclosed in Example 1, numerous cell culture media formulations and feed media were studied to maximize cell growth, cell viability, productivity, percent fucosylation, and Fc effector function of recombinant antibodies derived from a rat hybridoma cell line (e.g., YB2/0 cells) in culture.

In certain aspects, the present disclosure is directed to methods of making a recombinant protein in a rat hybridoma cell line comprising culturing the cells in a cell culture, wherein the rat hybridoma cells are cultured in basal media and feed media, appropriate for rat hybridoma cells.

In some aspects, the rat hybridoma cells are cultured in culture media that is chemically defined and animal-derived component-free (ADCF).

In some aspects, the rat hybridoma cells are cultured in CDM4Mab® (Cytiva) and feed media BalanCD CHO Feed 4® (Irvine Scientific). In some aspects, basal media CDM4Mab® and feed media BalanCD CHO Feed 4® are used in combination with other commercially available cell culture culture media or in combination with a cell culture medium that has been individually formulated for use with rat hybridoma cells. In some aspects, other basal media (aside from CDM4Mab®) are used in the rat hybridoma cell culture described herein. In some aspects, other feed media (aside from or in addition to BalanCD CHO Feed 4®) are used in the rat hybridoma cell culture described herein.

In some aspects, basal media, e.g., CDM4Mab®, is present in the cell culture starting on culture day 0 of the cell culture. In certain aspects, BalanCD CHO Feed 4® is added on culture day 3 of the cell culture.

In some aspects, the feed media, e.g., BalanCD CHO Feed 4® feed media, is added to the cell culture about every 48 to 72 hours. In some aspects, the feed media, e.g., BalanCD CHO Feed 4® feed media, is added to the cell culture about every 48 hours. In some aspects, the BalanCD CHO Feed 4® feed media is added to the cell culture about every 72 hours. In some aspects, glucose, glutamine, and/or a cholesterol lipid solution are added to supplement feed media BalanCD CHO Feed 4® in the cell culture.

In some aspects, the recombinant protein that is cultured in basal media CDM4Mab® and feed media BalanCD CHO Feed 4® is produced at an increased harvest titer relative to a cell culture that is not cultured in basal media CDM4Mab® and feed media BalanCD CHO Feed 4®. In some aspects, the harvest titer of the recombinant protein that is cultured in basal media CDM4Mab® and feed media BalanCD CHO Feed 4® is about 0.3 g/L to about 1.5 g/L. In some aspects, the harvest titer of the recombinant protein that is cultured in basal media CDM4Mab® and feed media BalanCD CHO Feed 4® is about 0.3 g/L to about 1.3 g/L. In some aspects, the harvest titer of the recombinant protein that is cultured in basal media CDM4Mab® and feed media BalanCD CHO Feed 4® is about 0.5 g/L to about 1.0 g/L. In some aspects, the harvest titer of the recombinant protein is about 1.0 g/L.

In some aspects, the percent fucosylation of the recombinant protein cultured in basal media CDM4Mab® and feed media BalanCD CHO Feed 4® is decreased relative to a cell culture that is not cultured in basal media CDM4Mab® and feed media BalanCD CHO Feed 4®. In some aspects, the percent fucosylation of the recombinant protein cultured in basal media CDM4Mab® and feed media BalanCD CHO Feed 4® is decreased to about 18% to about 73%. In some aspects, the percent fucosylation of the recombinant protein cultured in basal media CDM4Mab® and feed media BalanCD CHO Feed 4® is decreased to about 18% to about 40%. In some aspects, the percent fucosylation of the recombinant protein cultured in basal media CDM4Mab® and feed media BalanCD CHO Feed 4® is decreased to about 18% to about 30%. In some aspects, the percent fucosylation of the recombinant protein is decreased to about 18%.

In certain aspects, the rat hybridoma cell line cultured in basal media CDM4Mab® and feed media BalanCD CHO Feed 4® is a YB2/0 rat hybridoma cell line.

In some aspects, the recombinant protein produced in a rat hybridoma cell line cultured in basal media CDM4Mab® and feed media BalanCD CHO Feed 4® is an IgG1 glycoprotein. In some aspects, the recombinant protein produced in a rat hybridoma cell line cultured in basal media CDM4Mab® and feed media BalanCD CHO Feed 4® is a monoclonal antibody. In some aspects, the monoclonal antibody is a chimeric, humanized, or human antibody.

In some aspects, the monoclonal antibody produced in a rat hybridoma cell line cultured in basal media CDM4Mab® and feed media BalanCD CHO Feed 4® specifically binds an epitope of CD20. In some aspects, the monoclonal antibody produced in a rat hybridoma cell line cultured in basal media CDM4Mab® and feed media BalanCD CHO Feed 4® is anti-CD20 antibody TG-1101.

In some aspects, the percentage of FcγRIIIa-158V binding to the monoclonal antibody produced in a rat hybridoma cell line cultured in basal media CDM4Mab® and feed media BalanCD CHO Feed 4® is increased relative to the percentage of FcγRIIIa binding to a monoclonal antibody that was not cultured in basal media CDM4Mab® and feed media BalanCD CHO Feed 4®. In some aspects, the percent FcγRIIIa-158V binding is increased by about 5% to about 30%. In some aspects, the percent FcγRIIIa-158V binding is increased by about 20% to about 30%. In some aspects, the percent FcγRIIIa-158V binding is increased by about 20%. In certain aspects, the percent FcγRIIIa-158V binding is evaluated by surface plasmon resonance (SPR).

In some aspects, the percentage of the antibody-dependent cellular cytotoxicity (ADCC) activity of the monoclonal antibody produced in a rat hybridoma cell line cultured in basal media CDM4Mab® and feed media BalanCD CHO Feed 4® is increased relative to the percentage of ADCC activity of a monoclonal antibody that was not cultured in basal media CDM4Mab® and feed media BalanCD CHO Feed 4®. In some aspects, the percent ADCC activity is increased by about 10% to about 70%. In some aspects, the percent ADCC activity is increased by about 50% to about 70%. In some aspects, the percent ADCC activity is increased by about 60% to about 70%. In certain aspects, percent ADCC activity is evaluated by a cell-based bioassay.

In some aspects, the cell culture, cultured in basal media CDM4Mab® and feed media BalanCD CHO Feed 4®, is conducted in a bioreactor. In some aspects, the bioreactor is commercial-scale bioreactor (e.g., 15,000 L, 20,000 L, or 25,000 L capacity).

In some aspects, other cell culture process parameters, such as, e.g., pH and temperature control, as described herein, are shifted in conjunction with the use of basal media CDM4Mab® and feed media BalanCD CHO Feed 4® to optimally express a recombinant protein (e.g., antibody) in a rat hybridoma cell line (e.g., YB2/0).

### Harvesting the Recombinant Protein and Assessing Protein Quality, Titer, and Potency

In some aspects, the method of making a recombinant protein further comprises harvesting the recombinant protein produced by the rat hybridoma cells. In some aspects, harvesting of the cell culture will occur on culture day 12 or 13 of the cell culture, or when cell viability drops below 20%, whichever comes first.

In general, it will typically be desirable to isolate and/or purify proteins or antibodies expressed according to the present disclosure. In a preferred aspect, the expressed polypeptide or protein is secreted into the medium and thus cells and other solids may be removed, as by centrifugation or filtering for example, as a first step in the purification process.

Alternatively, the expressed polypeptide or protein is bound to the surface of the host cell. In this embodiment, the media is removed and the host cells expressing the polypeptide or protein are lysed as a first step in the purification process. Lysis of mammalian host cells can be achieved by any number of means well known to those of ordinary skill in the art, including physical disruption by glass beads, exposure to high pH conditions, exposure to freezing temperatures, and the addition of cell lysis buffers with detergents.

The polypeptide or protein may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin, or aprotinin may be added at any or all stages in order to reduce or eliminate degradation of the polypeptide or protein during the purification process. Protease inhibitors are particularly desired when cells must be lysed in order to isolate and purify the expressed polypeptide or protein. One of ordinary skill in the art will appreciate that the exact purification technique will vary depending on the character of the polypeptide or protein to be purified, the character of the cells from which the polypeptide or protein is expressed, and the composition of the medium in which the cells were grown.

In some aspects, the presently disclosed method further comprises purifying the recombinant protein by affinity chromatography and/or ion exchange chromatography. In some aspects, the affinity chromatography comprises protein A purification. In some aspects, the purified recombinant protein produced by the rat hybridoma cell is formulated into a pharmaceutically acceptable formulation.

In some aspects, after the expressed recombinant protein is harvested and purified from the cell culture, product quality is evaluated.

In some aspects, the quality of the purified recombinant protein is measured by SEC-HPLC, imaged capillary electrophoresis (ICIEF), and/or N-linked glycan analysis. In some aspects, the purified recombinant protein (e.g., monoclonal antibody) is measured for percent fucosylation by conducting an N-linked glycan analysis, as described, e.g., in Examples 1, 2, and 6.

In some aspects, the purified recombinant protein is a monoclonal antibody. In some aspects, the monoclonal antibody specifically binds an epitope of CD20 (also referred to herein as an “anti-CD20 antibody”). In some aspects, the anti-CD20 antibody specifically binds the same epitope as TG-1101.

In some aspects, the biological properties of the anti-CD20 antibodies made by the methods disclosed herein can be measured and described in an assay with the use of a comparison with a reference standard. In some aspects, the reference standard is a commercial reference standard. In some aspects, the commercial reference standard is RS-117808. RS-117808 (“antibody Ublituximab (TG-1101)”) was deposited according to the terms of the Budapest Treaty at the American Type Culture Collection (ATCC), located at 10801 University Boulevard, Manassas, VA 20110, received by the ATCC on Apr. 15, 2022, and assigned unofficial patent deposit number PTA-127294.

In some aspects, the reference standard is an anti-CD20 antibody. In certain aspects, the reference standard is GAZYVA (obinutuzumab), ARZERRA (ofatumumab), RITUXAN (rituximab), veltuzumab (IMMU-106), ZEVALIN (ibritumomab tiuxetan) or OCREVUS (ocrelizumab).

In some aspects, the monoclonal antibody is subject to one or more binding assays to evaluate CD20 binding activity (or other antibody binding, if the antibody is not an anti-CD20 antibody), FcγRIIIa-158V binding, and/or C1q binding. In some aspects, the recombinant antibody is assayed for antibody-dependent cell cytotoxicity (ADCC) biological activity/potency and/or complement dependent cytotoxicity (CDC) biological activity/potency, such as described in Examples 1 and 2. Additional assays to assess protein or antibody quality or function are well known to those skilled in the art.

In some aspects, the monoclonal antibody produced by the presently disclosed methods has a relative potency of 82% to 138% % in a cell-based CD20 binding activity bioassay compared to that of a commercial reference standard. In some aspects, the monoclonal antibody produced by the presently disclosed methods has a relative potency of 92% to 118%% in a cell-based CD20 binding activity bioassay compared to that of a commercial reference standard. In some aspects, the monoclonal antibody produced by the presently disclosed methods has a relative potency of 109% in a cell-based CD20 binding activity bioassay compared to that of a commercial reference standard. In some aspects, the percentage of CD20 binding is determined by a cell-based CD20 binding activity bioassay, such as the binding of anti-CD20 antibody to the CD20 expressing human mantle cell lymphoma cell line, Jeko-1.

In some aspects, the monoclonal antibody produced by the presently disclosed methods has a relative potency of 82% to 130% FcγRIIIa-158V binding relative to a commercial reference standard as measured by surface plasmon resonance (SPR). In some aspects, the monoclonal antibody produced by the presently disclosed methods has a relative potency of 76% to 130% FcγRIIIa-158V binding relative to a commercial reference standard as measured by surface plasmon resonance (SPR).

In some aspects, the monoclonal antibody has a KD value of 30 to 70 nM in an FcγRIIIa-158V binding assay as measured by surface plasmon resonance. In some aspects, the monoclonal antibody has a KD value of about 59 nM in an FcγRIIIa-158V binding assay as measured by surface plasmon resonance. In some aspects, the monoclonal antibody has a KD value of 500 to 1000 nM in an FcγRIIIa 158F binding assay as measured by surface plasmon resonance. In some aspects, the monoclonal antibody has a KD value of 760 nM in an FcγRIIIa 158F binding assay as measured by surface plasmon resonance. In some aspects, the monoclonal antibody has significantly higher binding affinity to FcγRIIIa 158V or FcγRIIIa 158F than the anti-CD20 antibody, rituximab.

In some aspects, the monoclonal antibody produced by the presently disclosed methods has a relative potency of 86 to 117% in a C1q binding assay as measured by ELISA compared to a commercial reference standard. In some aspects, the monoclonal antibody produced by the presently disclosed methods has a relative potency of 86% to 116% in a C1q binding assay as measured by ELISA compared to a commercial reference standard. In some aspects, the monoclonal antibody produced by the presently disclosed methods has a relative potency of 88 to 113% in a C1q binding assay as measured by ELISA compared to a commercial reference standard. In some aspects, the monoclonal antibody produced by the presently disclosed methods has a relative potency of about 99% in a C1q binding assay as measured by ELISA compared to a commercial reference standard.

In some aspects, the monoclonal antibody produced by the presently disclosed methods has higher percent antibody-dependent cellular cytotoxicity (ADCC) activity relative to a monoclonal antibody produced by a culturing process that does not employ the culture conditions of i) an initial temperature set point of about 37° C., a second temperature set point of about 35° C., and a third temperature set point of about 32.5° C.; ii) a culture pH between about 6.5 to about 7.55; and iii) a culture pCO₂ less than about 300 mmHg.

In some aspects, the monoclonal antibody produced by the presently disclosed methods induces greater cytotoxicity in a cell-based antibody-dependent cellular cytotoxicity (ADCC) assay compared to obinutuzumab, ofatumumab, rituximab, veltuzumab, ibritumomab tiuxetan, and/or ocrelizumab.

In some aspects, the monoclonal antibody produced by the presently disclosed methods has a relative potency of 90% to 163% in a cell-based ADCC assay compared to a commercial reference standard. In some aspects, the monoclonal antibody produced by the presently disclosed methods has a relative potency of about 117% in a cell-based ADCC assay compared to a commercial reference standard. In some aspects, the cell-based ADCC assay uses effector cells selected from CD16 effector cells and primary NK cells. In some aspects, the population performs in a cell-based ADCC using CD16 effector cells at more than 100% of that of a commercial reference standard.

In some aspects, the monoclonal antibody produced by the presently disclosed methods has a relative potency of 74% to 127% in a cell-based complement dependent cytotoxicity (CDC) assay compared to that of a commercial reference standard. In some aspects, the monoclonal antibody produced by the presently disclosed methods has a relative potency of 73% to 128% in a cell-based complement dependent cytotoxicity (CDC) assay compared to that of a commercial reference standard. In some aspects, the monoclonal antibody produced by the presently disclosed methods has a relative potency of 78 to 116% in a cell-based CDC assay compared to that of a commercial reference standard. In some aspects, the monoclonal antibody produced by the presently disclosed methods has a relative potency of about 91% in a cell-based CDC assay compared to that of a commercial reference standard.

In some aspects, the monoclonal antibody produced by any of the methods disclosed herein comprises: a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some aspects, the monoclonal antibody comprises a heavy chain having at least 95% identity to an amino acid sequence set forth in SEQ ID NO:7 and a light chain having at least 95% identity to an amino acid sequence set forth in SEQ ID NO:8.

In some aspects, the monoclonal antibody comprises a heavy chain having an amino acid sequence set forth in SEQ ID NO:7 and a light chain having an amino acid sequence set forth in SEQ ID NO:8. In some aspects, the monoclonal antibody comprises a heavy chain having an amino acid sequence set forth in SEQ ID NO:7 and a light chain having an amino acid sequence set forth in SEQ ID NO:9. In some aspects, the monoclonal antibody comprises a deletion of up to 5 N-terminal residues. In some aspects, the monoclonal antibody comprises a deletion of up to 10 N-terminal sequences.

In some aspects, the presently disclosed methods yields a recombinant protein (e.g., anti-CD20 antibody) harvest titer of about 0.5 g/L to about 1.5 g/L. In some aspects, the harvest titer is about 1.0 g/L to about 1.5 g/L.

In some aspects, the anti-CD20 antibody is TG-1101 or an antibody that binds the same epitope as TG-1101. In some aspects the anti-CD20 antibody is TG-1101.

### Commercial-Scale Production of a Recombinant Protein in Rat Hybridoma Cells

Commercial-scale (e.g., 10,000 L-25,000 L) production of a recombinant protein (e.g., anti-CD20 antibody) expressed in rat hybridoma cells is also provided. See, Example 3.

In some aspects, a method of making a recombinant protein in a culture of rat hybridoma cells at a commercial-scale is provided comprising the steps of: a) preparing and thawing a working rat hybridoma cell bank of the recombinant protein of interest; b) expanding a culture of the rat hybridoma cells from the cell bank by size and volume though a series of shake flasks (125 mL, 500 mL, 3 L, 3×3 L shake flasks, and 50 L cellbag) with a targeted seeding density of at least 0.30×10⁶ viable cells/mL; c) processing the cell culture through a series of seed bioreactors (120 L, 600 L, and 3,000 L) to further increase the volume and cell culture mass; d) inoculating the cell culture from the 3,000 L seed bioreactor into a 15,000 L production bioreactor; e) harvesting the cell culture supernatant from the production bioreactor; f) clarifying the recovered cells by continuous centrifugation followed by depth filtration; g) purifying the recombinant protein by Protein A capture column chromatography; and h) inactivating viral agents by solvent detergent viral inactivation (SDVI).

In some aspects, Protein A column chromatography is performed to purify the recovered recombinant protein and reduce process impurities such as cell culture components, HCP, and residual DNA, as well as to provide for viral safety. In some aspects, Protein A column chromatography was performed using MabSuRe Select resin (Cytiva) in bind/elute mode.

In some aspects, the packed column is assessed for HETP performance using sodium acetate/benzyl alcohol buffer. In some aspects, column operations are performed at 13 to 25° C. In some aspects, before loading, the column is sanitized with 0.5 M sodium hydroxide, flushed with WFI, and equilibrated with Equilibration Buffer (25 mM Tris, 25 mM NaCl, 5 mM EDTA, pH 7.1). In some aspects, the clarified harvest is mixed briefly and then loaded onto the column using a maximum of 21 g recombinant protein/L resin load and the column was washed with Wash 1 Buffer (equilibration buffer) followed by a second wash with a high salt, Wash 2 Buffer (25 mM Tris, 1.2 M NaCl, 5 mM EDTA, pH 7.1), followed by an additional wash using Wash 3 Buffer (equilibration buffer). In some aspects, the clarified harvest is loaded onto the column using a maximum of 36 g recombinant protein/L resin load. Bound recombinant protein is eluted 200-220 cm/h with elution buffer (25 mM sodium citrate, pH 3.6) using elution peak collection by A280, not to exceed 1.2 column volumes. In some aspects, bound recombinant protein is eluted 200-220 cm/h with elution buffer (25 mM sodium citrate, pH 3.6) using elution peak collection by A280, not to exceed 2.4 column volumes. The eluate is collected in a tank containing neutralization buffer (2.0M Tris, pH 7.5) and filtered via a 0.2 m filter before transfer to a different tank. The Protein A column is sanitized with 0.5M sodium hydroxide. Up to three cycles per batch may be run; if multiple cycles were required for the Protein A process, the column is re-equilibrated for the next cycle with equilibration buffer. After sanitization, the Protein A column is neutralized with equilibration buffer and stored at 13 to 25° C. in 200 mM sodium acetate, 2% benzyl alcohol, pH 5.0. The pooled (if more than one cycle), neutralized eluate is diluted with 5 mM Sodium Phosphate, pH 7.2 to a concentration of ≤10 g/L, and stored at 13-25° C. for ≤72 hours or at 2 to 8° C. for ≤11 days. In some aspects, the pooled (if more than one cycle), neutralized eluate is diluted with 5 mM Sodium Phosphate, pH 7.2 to a concentration of ≤10 g/L and stored at 13-25° C. for ≤24 hours.

In some aspects, the Protein A capture chromatography step is followed by a solvent detergent viral inactivation (SDVI) step to inactivate potential viral agents. In some aspects, the Protein A elution pool is diluted and treated with 3.5% (v/v) TnBP, 12% (w/v) polysorbate 80 and held at 24.0-26.0° C. for at least 120 minutes while mixing. The SDVI pool is filtered with a 0.2 μm filter before transfer to a different tank, where it is diluted with 5 mM sodium phosphate, pH 7.2 to 50 mOsm/kg, and the pH adjusted to 7.2 as needed. After pH adjustment, the pool is held at 13 to 25° C. for ≤30 hours before proceeding to the CEX column. In some aspects, the pool is held at 13 to 25° C. for ≤24 hours.

In some aspects, the method further comprises purification by cation exchange chromatography (CEX) and anion exchange chromatography (AEX).

In some aspects, further purification of the recombinant protein and removal of residual process impurities is performed using cation exchange column chromatography (CEX). In some aspects, CEX was performed using SP Sepharose Fast Flow (Cytiva) in bind/elute mode. In some aspects, the packed column is assessed for HETP performance using a sodium acetate/benzyl alcohol-containing buffer and all column operations are performed at 13 to 25° C. In some aspects, the column is sanitized with 0.5 M sodium hydroxide, rinsed with WFI, and equilibrated using equilibration buffer (20 mM sodium phosphate, pH 7.2). The viral inactivated/diluted solution is loaded onto the column at a maximum of 65 g/L resin load. The column is washed with Wash 1 Buffer (equilibration buffer) followed by a second wash with Wash 2 Buffer (equilibration buffer in the reverse direction). Bound recombinant protein is eluted using 20 mM sodium phosphate, 150 mM NaCl, pH 7.2 and elution peak collection by A280 monitoring. In some aspects, the eluate is filtered (0.2 μm) and stored at 13 to 25° C. for ≤72 hours or at 2 to 8° C. for ≤11 days. In some aspects, the eluate is stored at 13 to 25° C. for ≤24 hours. After elution, the column is stripped with 2 M NaCl followed by sanitization with 0.5 M sodium hydroxide. One cycle per batch was allowed. After completion, the column is sanitized (0.5 M sodium hydroxide) and stored in storage buffer (200 mM sodium acetate, 2% benzyl alcohol, pH 5.0).

In some aspects, further purification of the recombinant protein is performed using anion exchange membrane chromatography. In some aspects, AEX is performed using a Mustang Q (Pall Corporation) membrane absorber (MA) filter in flow-through mode. In some aspects, the membrane was single use (i.e., each individual membrane cannot be reused) and several membrane capsules may be used per batch at the appropriate loading level. In some aspects, eluate from the CEX step is diluted with 20 mM sodium phosphate, pH 8.0, followed by the adjustment of the pH to 8.0. The concentration was determined and the number of cycles calculated based on protein concentration such that the loading was 200 to 700 g recombinant protein/L membrane load. The membrane is sanitized with 0.5 M sodium hydroxide, flushed with 2 M NaCl then with WFI, before being equilibrated with Equilibration Buffer (20 mM sodium phosphate, 75 mM NaCl, pH 8.0) in preparation for the load. After loading, the membrane is chased with 20 mM sodium phosphate, 75 mM NaCl, pH 8.0, to maximize recovery. The collected flow-through containing product from all cycles is filtered (0.5/0.2 μm), diluted to ≤6 g/L with 75 mM sodium citrate, 312 mM NaCl, pH 6.0, and the pH is adjusted to 6.8. In some aspects, the adjusted AEX pool is stored at 13 to 25° C. for ≤72 hours or at 2 to 8° C. for ≤11 days. In some aspects, the adjusted AEX pool is stored at 13 to 25° C. for ≤24 hours.

In some aspects, the method further comprised performing viral filtration (VF) to remove potential viruses, including small viruses such as parvovirus. In some aspects, VF is performed by filtering through a Viresolve prefilter in series with a Viresolve Pro viral filter (Millipore Sigma), at an operating temperature Target Range of 13-25° C. The process used sufficient filters to meet the loading limit. To perform the filtration, the filters are set up in series and flushed with WFI, integrity tested, then sanitized with 0.5 M sodium hydroxide. This was followed by flushing with equilibration buffer (25 mM sodium citrate, 154 mM NaCl, pH 6.5). The filtered Mustang Q membrane flow-through was processed through the viral reduction filters.

Protein concentration was determined and used to confirm the membrane load ratio was ≤600 g/m². After loading, the membrane was chased with equilibration buffer and post-use integrity testing was performed. In some aspects, viral filtrate was stored at 13 to 25° C. for ≤72 hours or at or at 2 to 8° C. for ≤11 days. In some aspects, viral filtrate was stored at 13 to 25° C. for ≤33 hours.

In some aspects, commercial-scale production is in a 10,000 L to 25,000 L production bioreactor. In some aspects, commercial-scale production is in a 15,000 L production bioreactor. In some aspects, the commercial-scale production is operated in fed-batch mode.

In some aspects, a drilled hole (10) gas sparger with 4.0 mm orifice diameter is used in the 15,000 L production bioreactor.

In some aspects, the method further comprises ultrafiltration/diafiltration (UFDF).

In some aspects, the method further comprises preparing a bulk drug substance formulation comprising the recombinant protein, comprising adding polysorbate 80 in formulation buffer to prepare a bulk drug substance formulation. In some aspects, the method further comprises subjecting the bulk drug substance formulation to 0.2 μm filtration.

In some aspects, the method further comprises filling a 6 L bag to a target fill volume of 5.50 L of the bulk drug substance formulation and storing the bulk drug substance formulation at ≤−35° C.

In some aspects, the recombinant protein in the bulk drug substance formulation is formulated into a pharmaceutically acceptable formulation.

In some aspects, the method further comprises testing unprocessed bulk harvest from the 15,000 L production bioreactor for microbial and viral adventitious agents and removing the microbial and viral adventitious agents from the 15,000 L production bioreactor.

In some aspects, the rat hybridoma cells are YB2/0 cells.

In some aspects, the recombinant protein produced by the manufacturing method is a monoclonal antibody. In some aspects, the monoclonal antibody binds an epitope of CD20 (i.e., is an anti-CD20 antibody). In some aspects, the monoclonal antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

In some aspects, the monoclonal antibody comprises a heavy chain having at least 95% identity to an amino acid sequence set forth in SEQ ID NO:7; and a light chain having at least 95% identity to an amino acid sequence set forth in SEQ ID NO: 8.

In some aspects, the monoclonal antibody comprises a heavy chain having an amino acid sequence set forth in SEQ ID NO:7; and a light chain having an amino acid sequence set forth in SEQ ID NO:8.

In some aspects, the monoclonal antibody comprises a heavy chain having an amino acid sequence set forth in SEQ ID NO:7; and a light chain having an amino acid sequence set forth in SEQ ID NO:9.

In some aspects, the monoclonal antibody is TG-1101 or an antibody that binds the same epitope as TG-1101. In some aspects, the monoclonal antibody is TG-1101. As described below, in some aspects, the monoclonal antibody produced by the methods disclosed herein contains a unique glycosylation signature.

### Rat Hybridoma Master Cell Bank and Working Cell Bank

Also provided herein are rat hybridoma master cell bank (MCB) compositions and rat hybridoma working cell bank (WCB) compositions, which can be used to make the recombinant proteins (e.g., monoclonal antibodies) disclosed herein. See, Example 3. The MCB and WCB, as disclosed herein, were phenotypically stable based on e.g., cell growth, harvest titer, productivity, and product quality, for at least 35, at least 40, at least 45, at least 50, at least 55, at least 60, at least 65, at least 70, and at least 75 cell generations. In some aspects, the MCB and WCB, as disclosed herein, were phenotypically stable based on e.g., cell growth, harvest titer, productivity, and product quality, for up to 71 cell generations. Id.

In some aspects, the rat hybridoma MCB provided herein comprises a recombinant protein having at least two, at least three, at least four, at least five, at least six, at least seven, at least eight, at least nine, or all of the following parameters: i) peak viable cell density of about 11 to about 13×106 cells/mL; ii) harvest titer of about 650 to about 720 mg/L; iii) percent fucosylation of about 30% to about 38%; iv) about 97% to about 99% monomers as detected by size exclusion chromatography (SEC); v) about 1.5% to about 2% dimers as detected by SEC; vi) undetectable to about 3% level of aggregates as detected by SEC; vii) undetectable to about 1% level of fragments as detected by SEC; viii) about 25% to about 30% acidic isoforms as detected by imaged capillary isoelectric focusing (iCIEF); ix) about 38% to about 49% main isoforms as detected by iCIEF; and/or x) about 20% to about 36% basic isoforms as detected by iCIEF.

In some aspects, the rat hybridoma MCB provided herein comprises a recombinant protein having at least two, at least three, at least four, at least five, at least six, at least seven, at least eight, at least nine, or all of the following parameters: i) peak viable cell density of about 11 to about 13×106 cells/mL; ii) harvest titer of about 650 to about 720 mg/L; iii) percent fucosylation of about 30% to about 38%; iv) about 97% to about 99% monomers as detected by size exclusion chromatography (SEC); v) about 1.5% to about 2% dimers as detected by SEC; vi) undetectable to about 3% level of aggregates as detected by SEC; vii) undetectable to about 1% level of fragments as detected by SEC; viii) about 25% to about 30% acidic isoforms as detected by imaged capillary isoelectric focusing (iCIEF); ix) about 38% to about 49% main isoforms as detected by iCIEF; and/or x) about 20% to about 36% basic isoforms as detected by iCIEF.

In some aspects, the rat hybridoma WCB provided herein comprises a recombinant protein having at least two of the following parameters: i) peak viable cell density that is about 11 to about 28×106 cells/mL; ii) harvest titer that is about 420 to about 1280 mg/L; iii) percent fucosylation of about 18% to about 40%; iv) about 97% to about 99% monomers as detected by size exclusion chromatography (SEC); v) about 1% to about 2% dimers as detected by SEC; vi) undetectable to about 2% level of aggregates as detected by SEC; vii) undetectable to about 1% level of fragments as detected by SEC; viii) about 19% to about 31% acidic isoforms as detected by imaged capillary isoelectric focusing (iCIEF); ix) about 34% to about 62% main isoforms as detected by iCIEF; and/or x) about 14% to about 38% basic isoforms as detected by iCIEF.

In some aspects, the rat hybridoma WCB provided herein comprises a recombinant protein having at least two of the following parameters: i) peak viable cell density that is about 11 to about 28×106 cells/mL; ii) harvest titer that is about 420 to about 1280 mg/L; iii) percent fucosylation of about 18% to about 40%; iv) about 97% to about 99% monomers as detected by size exclusion chromatography (SEC); v) about 1% to about 2% dimers as detected by SEC; vi) undetectable to about 2% level of aggregates as detected by SEC; vii) undetectable to about 1% level of fragments as detected by SEC; viii) about 19% to about 31% acidic isoforms as detected by imaged capillary isoelectric focusing (iCIEF); ix) about 34% to about 62% main isoforms as detected by iCIEF; and/or x) about 14% to about 38% basic isoforms as detected by iCIEF.

In some aspects, the rat hybridoma cells in the MCB composition or the WCB composition are YB2/0 cells. In some aspects, the recombinant protein to be produced from the MCB or WCB composition is a monoclonal antibody. In some aspects, the monoclonal antibody is an anti-CD20 antibody.

In some aspects, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some aspects, the monoclonal antibody comprises a heavy chain having at least 95% identity to an amino acid sequence set forth in SEQ ID NO:7; and a light chain having at least 95% identity to an amino acid sequence set forth in SEQ ID NO: 8. In some aspects, the monoclonal antibody comprises a heavy chain having an amino acid sequence set forth in SEQ ID NO:7; and a light chain having an amino acid sequence set forth in SEQ ID NO:8. In some aspects, the monoclonal antibody comprises a heavy chain having an amino acid sequence set forth in SEQ ID NO:7; and a light chain having an amino acid sequence set forth in SEQ ID NO:9.

Also provided is a method of making a recombinant protein by using the MCB or WCB composition, as disclosed herein. In some aspects, the recombinant protein is a monoclonal antibody. In some aspects, the monoclonal antibody is an anti-CD20 antibody. In some aspects, the anti-CD20 antibody comprises: a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6. In some aspects, the monoclonal antibody comprises a heavy chain having at least 95% identity to an amino acid sequence set forth in SEQ ID NO:7; and a light chain having at least 95% identity to an amino acid sequence set forth in SEQ ID NO: 8. In some aspects, the monoclonal antibody comprises a heavy chain having an amino acid sequence set forth in SEQ ID NO:7; and a light chain having an amino acid sequence set forth in SEQ ID NO:8. In some aspects, the monoclonal antibody comprises a heavy chain having an amino acid sequence set forth in SEQ ID NO:7; and a light chain having an amino acid sequence set forth in SEQ ID NO:9.

### Recombinant Proteins and Compositions

Recombinant proteins made according to any of the methods disclosed herein are provided. In some aspects, the recombinant protein is a monoclonal antibody. In some aspects, the monoclonal antibody is an antibody that binds an epitope of CD20. In some aspects, the monoclonal antibody is TG-1101 (TG Therapeutics, Inc.) or an antibody that binds the same epitope as TG-1101. In some aspects, the monoclonal antibody is TG-1101.

In one aspect, the present disclosure provides a composition comprising a recombinant protein produced in rat hybridoma cells (e.g., YB2/0) by any of the methods disclosed herein. In some aspects, the recombinant protein is a monoclonal antibody. In some aspects, the monoclonal antibody targets the CD20 antigen (i.e., is an anti-CD20 antibody). In some aspects, the monoclonal antibody is TG-1101 or an antibody that binds the same epitope as TG-1101. In some aspects, the monoclonal antibody is TG-1101.

In some aspects, the composition (e.g., comprising an anti-CD20 antibody) produced by the methods disclosed herein, has a unique glycosylation/N-glycan profile characterized by, e.g., between about 20% and about 40% fucosylated glycans and/or between about 10% and about 20% galactosylated glycans.

In some aspects, the monoclonal antibody comprises an N-glycan profile comprising one or both of the following: i. about 10 to 20% galactosylated glycans; and/or ii. about 23 to 36% fucosylated glycans.

In some aspects, the monoclonal antibody comprises an N-glycan profile comprising about 10 to 20% galactosylated glycans and about 23 to 36% fucosylated glycans.

In some aspects, the monoclonal antibody comprises an N-glycan profile comprising about 36% fucosylated glycans.

In some aspects, the monoclonal antibody comprises an N-glycan profile comprising about 16 to 18% galactosylated glycans.

In some aspects, the N-glycan profile comprises about 17% galactosylated glycans.

In some aspects, the monoclonal antibody comprises an N-glycan profile comprising at least about 10% bisecting N-glycans. In some aspects, the N-glycan profile comprises about 12% to 30% bisecting N-glycans. In some aspects, the N-glycan profile comprises about 18% bisecting N-glycans.

In some aspects, the monoclonal antibody comprises an N-glycan profile comprising less than 5% sialylated glycans. In some aspects, the N-glycan profile comprises less than 4%, 3%, 2.5%, 2%, 1%, or 0.5% sialylated glycans. In some aspects, the N-glycan profile comprises no detectable amount of sialylated glycan.

In some aspects, the monoclonal antibody comprises an N-glycan profile comprising 0.1% to 1.5% Man5 N-glycan. In some aspects, the N-glycan profile comprises 0.4% to 0.7% Man5 N-glycan. In some aspects, the N-glycan profile comprises about 0.6% Man5 N-glycan. In some aspects, Man5 N-glycan is the only high mannose species in the N-glycan profile.

In some aspects, the cell culture composition comprising a recombinant protein, monoclonal antibody, anti-CD20 antibody, or TG-1101, produced in rat hybridoma cells by any of the methods disclosed herein, is a pharmaceutical composition, wherein the recombinant protein, monoclonal antibody, anti-CD20 antibody, or TG-1101, is formulated together with a pharmaceutically acceptable carrier.

As used herein, the phrase “pharmaceutical composition” refers to a composition that is acceptable for pharmaceutical administration, such as to a human being. Such a composition can include substances that are impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s).

In some aspects, the pharmaceutical composition comprises one or more of the following: sodium chloride, trisodium citrate dehydrate, polysorbate 80, and hydrochloric acid.

Also provided are methods of treating a hematological malignancy in a subject in need thereof by administering the recombinant proteins or monoclonal antibodies (e.g., anti-CD20 antibodies) made according to any of the methods disclosed herein. In some aspects, the hematological cancer is lymphoma, leukemia, or myeloma. In some aspects, the hematological cancer is selected from B-cell lymphoma, acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), mantle cell lymphoma (MCL), follicular lymphoma (FL), Waldenstrom's macroglobulinemia (WM), diffuse large B-cell lymphoma (DLBCL), marginal zone lymphoma (MZL), hairy cell leukemia (HCL), Burkitt's lymphoma (BL), Richter's transformation, or primary central nervous system lymphoma (PCNSL).

Also provided are methods of treating an autoimmune disorder in a subject in need thereof by administering the recombinant proteins or monoclonal antibodies (e.g., anti-CD20 antibodies) made according to any of the methods disclosed herein. In some aspects, the autoimmune disease is multiple sclerosis, psoriasis, rheumatoid arthritis, vasculitis, inflammatory bowel disease, dermatitis, osteoarthritis, inflammatory muscle disease, allergic rhinitis, vaginitis, interstitial cystitis, scleroderma, osteoporosis, eczema, allogenic or xenogenic transplantation (organ, bone marrow, stem cells and other cells and tissues), graft rejection, graft-versus-host disease, lupus erythematosus, inflammatory disease, type 1 diabetes, pulmonary fibrosis, dermatomyositis, Sjogren's syndrome, thyroiditis (e.g., Hasimoto's and autoimmune thyroiditis), myasthenia gravis, autoimmune hemolytic anemia, cystic fibrosis, chronic relapsing hepatitis, primary biliary cirrhosis, allergic conjunctivitis, atopic dermatitis, chronic obstructive pulmonary disease, glomerulonephritis, neuroinflammatory diseases, or uveitis.

In some aspects, the autoimmune disease is multiple sclerosis. In some aspects, the multiple sclerosis is a relapsing form of multiple sclerosis. In certain embodiments, the relapsing form of multiple sclerosis is a clinically isolated syndrome (CIS); relapsing-remitting MS (RRMS); active secondary progressive MS (SPMS); or primary progressive MS (PPMS). In preferred aspects, the subject is a human.

The following examples are offered by way of illustration, and not by way of limitation.

### EXAMPLES

In the following Examples, cell culture processes with which to control growth and productivity of an anti-CD20 IgG1 monoclonal antibody (TG-1101), expressed by YB2/0 rat hybridoma cells, are exemplified through, e.g., choice of cell culture media (Example 1), culture pH, temperature, and pCO2 control methodologies (Example 2). Example 3 exemplifies 15,000 L commercial-scale production of an anti-CD20 antibody in rat hybridoma cells. Example 4 exemplifies the detection and removal of viruses or other adventitious agents in a 15,000 commercial-scale production process. Example 5 exemplifies a calculated pH process parameter, “integrated pH2 difference,” and its impact on harvest titer, percent fucosylation, and integrated viable cell density (IVCD). Finally, Example 6 exemplifies the unique glycosylation profile of the anti-CD20 antibody produced by the methods disclosed herein.

Increased process productivity was elicited while also decreasing protein fucosylation levels, and increasing Fc effector activity of the antibody. The demonstrated process for controlling both productivity and product quality is shown using this unique, uncommonly used mammalian expression system. The observed results are unexpected and surprising—Although YB2/0 cells have been shown to support low levels of fucosylation (Kanda, Y. et al., Biotechnol Bioeng 94:680-688 (2006)), it has not been shown with these cells that fucosylation levels can be directed lower and productivity directed higher through cell culture process means alone. Further, production of an anti-CD20 antibody in rat hybridoma cells on a commercial-scale has never been demonstrated, let alone one having a unique glycosylation signature.

### Example 1: Increasing Anti-CD20 Antibody Productivity Expressed by a YB2/0 Rat Hybridoma Cell Line Through Choice of Cell Culture Media

### Materials and Methods

### Cell Culture

A YB2/0 rat hybridoma cell line (ATCC CRL 1662) was adapted to 10 commercially available cell culture media (Table 2). Following scale up from thaw in static T-flasks in EM-SF2-P5-H4 media, cells were passaged into 125 mL shake flasks containing each of the 10 new media (at 2-8° C.), and placed into humidified incubators at 37° C. agitating at 90 RPM with a % CO₂ corresponding to the manufacturer's recommendations for each medium. The cultures were passaged on a daily basis at a target seeding density of 0.5×10⁶ until either the cell viability dropped below 60%, in which case the culture was terminated, or until the growth rate viability was steady for 5 days, after which the cells were considered adapted. To determine optimal passage strategy, a growth curve was performed on the best performing medium. All media were supplemented with 1 mL/L of 1000× Cholesterol as well as 6 mM L-Glutamine.

TABLE 2
Basal media evaluated for growth of YB2/0 cell line

| Basal medium | Supplier |
|---|---|
| Hycell Medium | GE |
| IS CHO-SD G10.6 Medium | Irvine Scientific |
| CD Hybridoma | Thermo Fisher |
| PFHM-II Protein Free Hybridoma | Thermo Fisher |
| Ex-Cell CD Hybridoma | SAFC |
| UltraDOMA-PF Hybridoma | Lonza |
| ProDOMA 1 | Lonza |
| IS MAB-CD | Irvine Scientific |
| LM \| CDM4Mab | Cytiva/GE |
| ActiCHO-P | GE |

Following the medium adaptation period in 125 mL shake flasks, cells were determined to have successfully adapted to 4 new media: CDM4Mab, CD Hybridoma, Hycell, and ProDoma 1. To obtain 7 unique basal media conditions for further screening against 6 commercially available feeds (Table 3), the top performing medium in terms of growth and viability (CDM4Mab®) was blended in a 50:50 ratio with the other 3 successful media. A definitive screening design of experiment (DOE) was used to identify the top performing combination of growth and feed media when run in fed batch mode using the Ambr15 cell culture platform (Sartorius). Additionally, one vessel was run with historical basal medium and feed as a control, and 5 vessels were run with the top performing new medium (CDM4Mab®) supplemented with varying concentrations of both a fatty acid supplement and Long R3 IGF-1 supplement. Process parameters used for all vessels are shown in Table 4. The measured outputs of each parameter were viable cell density, cell viability, antibody titer, and the glycosylation profile of the expressed mAb on the day of harvest for the top 12 conditions (including the control).

TABLE 3
Feed media evaluated for growth and
productivity of YB2/0 cell line

| Feed medium | Supplier |
|---|---|
| EX-CELL Advanced CHO Feed 1 | SAFC |
| (with Glucose) | |
| Cell Boost 6 | GE |
| CHO CD Efficient Feed A | Thermo Fisher |
| BalanCD CHO feed 4 | Irvine Scientific |
| Cell Boost 7A and b | GE |
| Cell Boost 3 | GE |

TABLE 4
Cell culture process conditions used for screening different cell culture media

| | | Target | |
|---|---|---|---|
| | | Operating | |
| Parameter | Set-Point | Range | Comment |
| Target Seeding | 0.50 | 0.40-0.60 | N/A |
| Density (×10⁶ VCD) | | | |
| Temperature set point | 37.0 | 36.0-38.0 | Temp shift per current process |
| | | | to 35.0 on Day 1 |
| pH Set Point | Day 0: 7.1 ± 0.3 | 6.8-7.4 | Acidic control: sparged CO₂ |
| | Day 3: 6.75 ± 0.15 | 6.6-6.9 | Basic Control: 10% NaHCO₃ |
| | Day 8: 6.85 ± 0.15 | 6.7-7.0 | pH shifts per current process |
| Agitation, RPM | 784 | ±10 | Constant agitation |
| DO level, % | 40% | Minimum 20% | Overlay oxygen on demand |
| saturation | | | |
| with air | | | |
| Glutamine feed | 6 mM Day 3 | N/A | 150% current process |
| | 3 mM EOD | | |
| | starting day 5 | | |
| Sparger Type | N/A | N/A | Head space only |
| Harvest Criteria | Day 14 or | N/A | N/A |
| | Viability <50% | | |
| Feeding Strategy | Feed dependent | TBD | Feed dependent |

### Protein Analytic Characterization

### N-Glycans

N-linked glycans are cleaved from the product by enzymatic deglycosylation using PNGase F and labeled with 2-aminobenzamide (2-AB), a fluorescent compound. The labeled glycans are resolved using a hydrophilic interaction partition mode ultra-performance liquid chromatography (HILIC-UPLC) column equipped with a fluorescence detector (fluorescence excitation at 360 nm and emission at 428 nm). Blank, glycans from reference standard, and glycan standards (human IgG N-linked glycan library) are also injected to assess system suitability. Peak identification from the resulting test sample chromatograms are identified based on retention time and relative to peaks in the glycan standard which have been confirmed by mass spectrometry.

### ADCC Potency Assay

The ADCC assay is a cell-based assay using CD20 expressing Jeko-1 cells, a human mantle cell lymphoma cell line, and Eurofins-DiscoverX “KILR CD16a effector cells,” which are single donor-derived human CD8+ T-lymphocytes engineered to express CD16 (FcγRIII) on their plasma membrane surface. The target cell lysis mediated by antibody samples is measured. Dose response curves are modeled using 4 parameter logistic regression (4PL), and results are reported as % potency relative to the reference standard.

### CDC Potency Assay

The CDC assay is a cell-based assay using the CD20 expressing Jeko-1 cells and rabbit serum as the source of complement. CDC mediated cell lysis is measured. Dose response curve is modeled using the 4PL equation, and results are reported as % potency relative to the reference standard.

### CD20 Binding Activity

In the CD20 binding activity assay, the binding of anti-CD20 antibody to the CD20 expressing human mantle cell lymphoma cell line, Jeko-1, is evaluated using electrochemiluminescence (Meso Scale Discovery). Dose response curves are modeled using 4PL equation, and results are reported as % potency relative to the reference standard.

### FcγRIIIa Binding

Binding of anti-CD20 antibody samples to FcγRIIIa is evaluated by surface plasmon resonance (SPR). The method follows a direct binding assay methodology where the Fc receptor (ligand) is directly immobilized onto the appropriate flow cell on a sensor chip surface and the analyte is injected over the chip to assess binding. The high affinity allele of FcγRIIIa (158V) is determined.

The equilibrium dissociation constants (KD) and the relative affinities of each sample relative to the reference standard is determined for each receptor. The rates of change of the SPR signal are analyzed using a 1:1 Langmuir model to yield apparent rate constants for the association and dissociation phases of the reaction, and equilibrium dissociation constants. Results are reported as % potency relative to the reference standard for the FcγRIIIa-158V variant.

### C1q Binding

Binding of an anti-CD20 antibody to C1q is evaluated using a C1q ELISA based method. The dose-response binding is modeled by weighted nonlinear regression using a 4-parameter logistic fit and the results are reported as % potency relative to the reference standard.

### Results

After successfully adapting cells to new media, adapted cells were used to inoculate Ambr 15 bioreactors (Sartorius). The purpose of the study was to identify growth media and feed media combinations that resulted in approximately double the titer and similar product quality of the fed-batch process using PROEMS-02 growth medium and Cell Boost 3 feed medium (˜320 mg/L, 40-60% fucosylation). The study was carried out using the process conditions shown above in Methods.

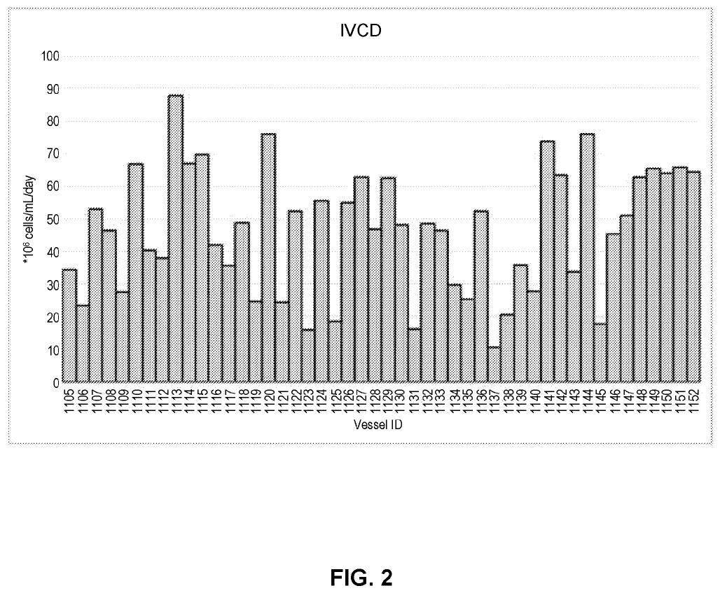

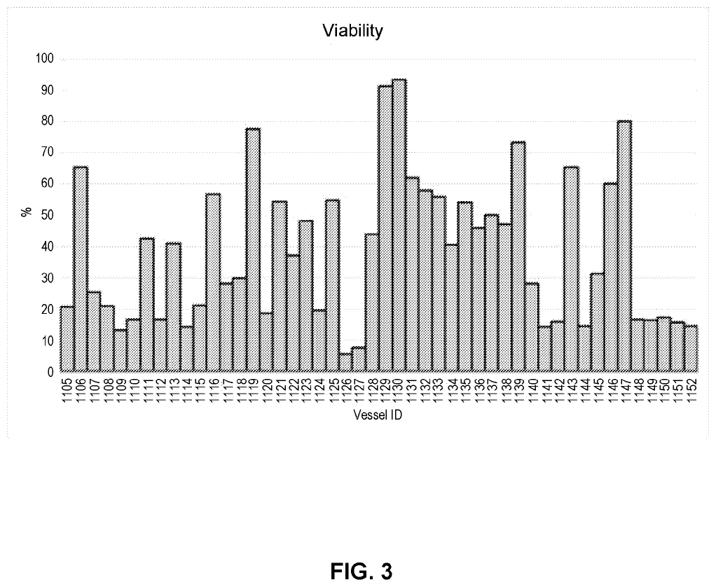

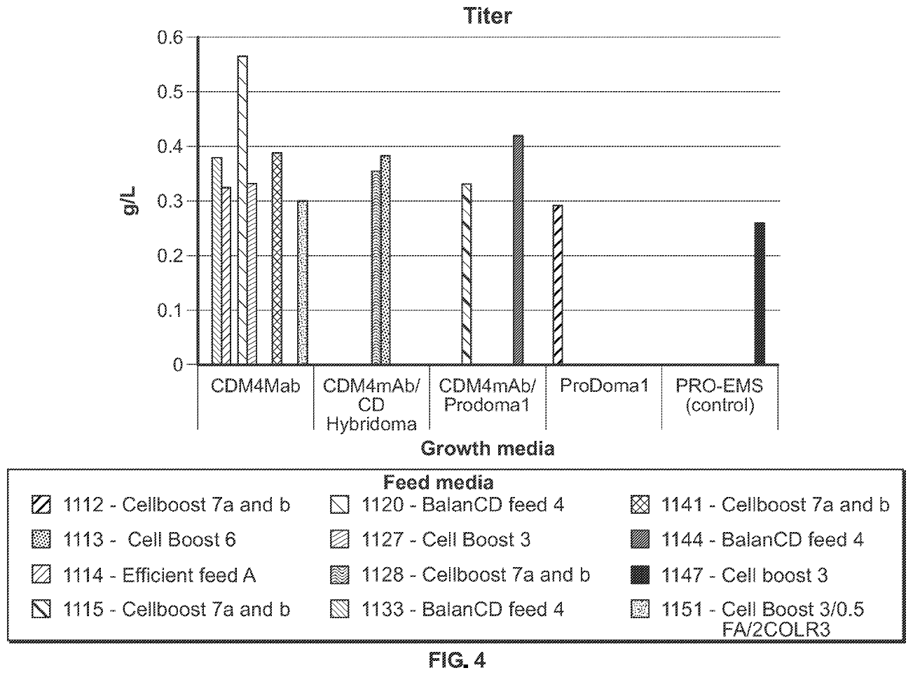

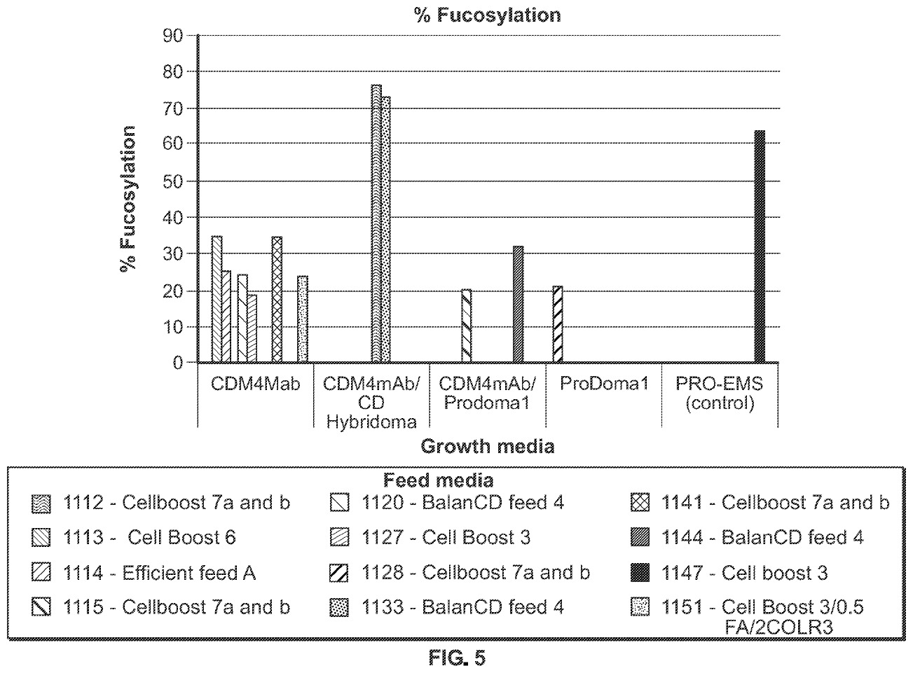

A wide range of cell growth performance was seen across the different growth media and feed media combinations (Table 5), with Day 10 integral of viable cell density (IVCD) results ranging from 10-87×10⁶ viable cells/mL/day (FIG. 2), and Day 10 cell viabilities ranging from 7.5-93.5% (FIG. 3). FIG. 4 depicts the top 11 media conditions (plus the control condition: PRO-EMS-02/Cell Boost 3) in terms of harvest titer, and FIG. 5 depicts the percent (%) fucosylation results of the top 11 media conditions plus the control. All 11 conditions outperformed the control media in terms of titer, and CDM4Mab was best represented amongst the top 11 new growth media and feed media combinations. The combination of CDM4Mab media with BalanCD CHO Feed 4 produced the highest titer of 0.563 g/L, and represents the only media/feed combination that came close to achieving a 2-fold increase in titer.

In terms of product quality, CDM4Mab/BalanCD CHO Feed 4 demonstrated significantly lower % fucosylation (˜24%) compared to the range supported by the control conditions (i.e., 40-60%). In addition, when CDM4Mab growth media was blended in a 50:50 ratio with CD Hybridoma media and combined with the feed media BalanCD CHO Feed 4, the result was still a top performing condition in terms of titer (˜0.38 g/L), while overall % fucosylation was increased to ˜72%. This suggests that while keeping the feed medium consistent (BalanCD CHO Feed 4), the % fucosylation results can be changed on the mAb by varying the ratio of different growth media.

TABLE 5
Cell Culture Media and Feed Media Combinations Evaluated

| Vessel | | |
|---|---|---|
| ID | Growth Medium | Feed Medium |
| 1105 | ProDoma 1 | Efficient feed A |
| 1106 | Hycell | Excell CHO feed 1 |
| 1107 | CDM4mAb/Prodomal | Cell boost 3 |
| 1108 | ProDoma 1 | Excell CHO feed 1 |
| 1109 | ProDoma 1 | BalanCD feed 4 |
| 1110 | CDM4mAb/Prodomal | Excell CHO feed 1 |
| 1111 | CDM4mAb/CDHybridoma | Efficient feed A |
| 1112 | ProDoma 1 | Cellboost 7a and b |
| 1113 | CDM4Mab | Cell Boost 6 |
| 1114 | CDM4Mab | Efficient feed A |
| 1115 | CDM4mAb/Prodomal | Cellboost 7a and b |
| 1116 | CDM4mAb/CDHybridoma | Cell boost 3 |
| 1117 | CDM4mAb/Hycell | BalanCD feed 4 |
| 1118 | CDM4mAb/Hycell | Excell CHO feed 1 |
| 1119 | CD Hybridoma | Cell boost 3 |
| 1120 | CDM4Mab | BalanCD feed 4 |
| 1121 | CD Hybridoma | BalanCD feed 4 |
| 1122 | CDM4mAb/CDHybridoma | Cell Boost 6 |
| 1123 | Hycell | Cell Boost 6 |
| 1124 | CDM4mAb/Prodomal | Cell Boost 6 |
| 1125 | CD Hybridoma | Efficient feed A |
| 1126 | CDM4mAb/Prodomal | Efficient feed A |
| 1127 | CDM4Mab | Cell boost 3 |
| 1128 | CDM4mAb/CDHybridoma | Cellboost 7a and b |
| 1129 | CDM4mAb/Hycell | Cell boost 3 |
| 1130 | CDM4mAb/Hycell | Cell Boost 6 |
| 1131 | Hycell | Cell boost 3 |
| 1132 | CDM4mAb/Hycell | Cellboost 7a and b |
| 1133 | CDM4mAb/CDHybridoma | BalanCD feed 4 |
| 1134 | ProDoma 1 | Cell Boost 6 |
| 1135 | CD Hybridoma | Cellboost 7a and b |
| 1136 | CDM4mAb/CDHybridoma | Excell CHO feed 1 |
| 1137 | Hycell | Efficient feed A |
| 1138 | Hycell | Cellboost 7a and b |
| 1139 | CD Hybridoma | Cell Boost 6 |
| 1140 | ProDoma 1 | Cell boost 3 |
| 1141 | CDM4Mab | Cellboost 7a and b |
| 1142 | CDM4Mab | Excell CHO feed 1 |
| 1143 | CD Hybridoma | Excell CHO feed 1 |
| 1144 | CDM4mAb/Prodomal | BalanCD feed 4 |
| 1145 | Hycell | BalanCD feed 4 |
| 1146 | CDM4mAb/Hycell | Efficient feed A |
| 1147 | PRO-EMS | Cell boost 3 |
| 1148 | CDM4Mab | Cell Boost 3/0.25 FA/100LR3 |
| 1149 | CDM4Mab | Cell Boost 3/0.375 FA/150LR3 |
| 1150 | CDM4Mab | Cell Boost 3/0.50 FA/100LR3 |
| 1151 | CDM4Mab | Cell Boost 3/0.5 FA/200LR3 |
| 1152 | CDM4Mab | Cell Boost 3/0.25 FA/200LR3 |

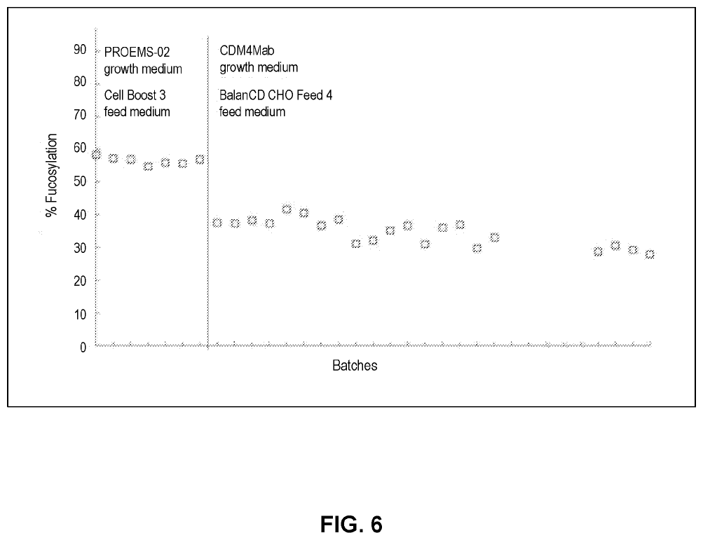

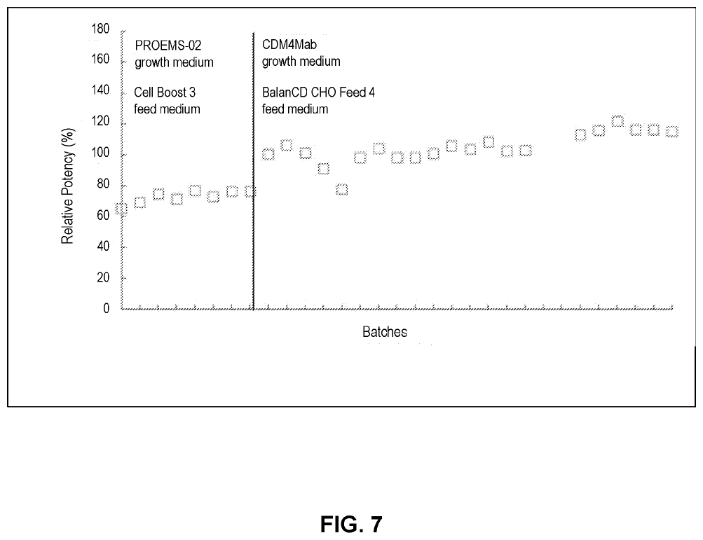

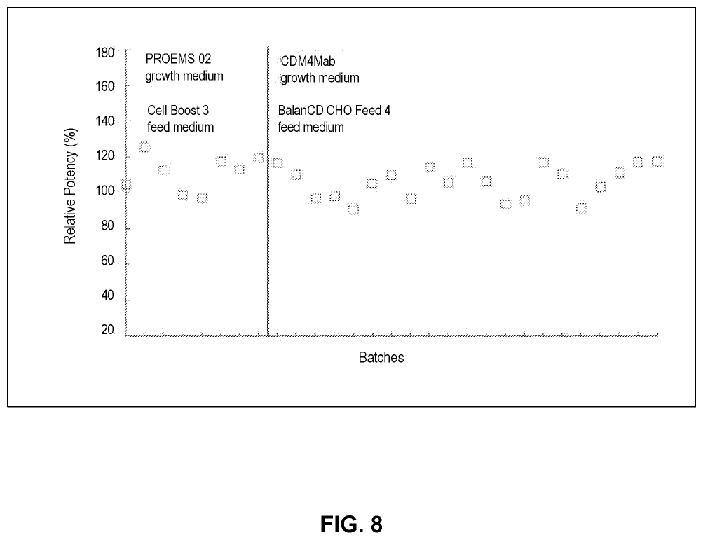

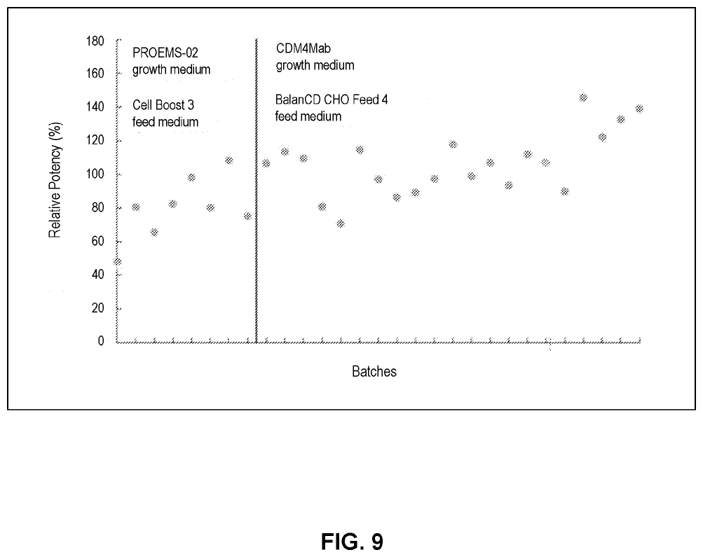

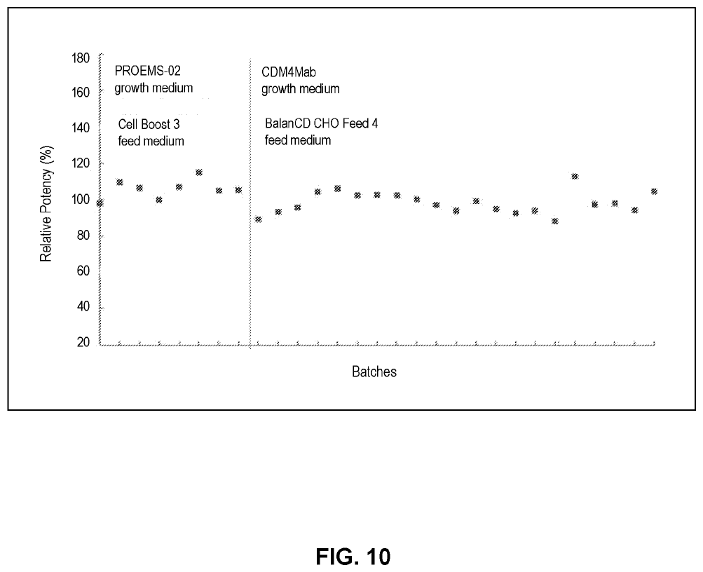

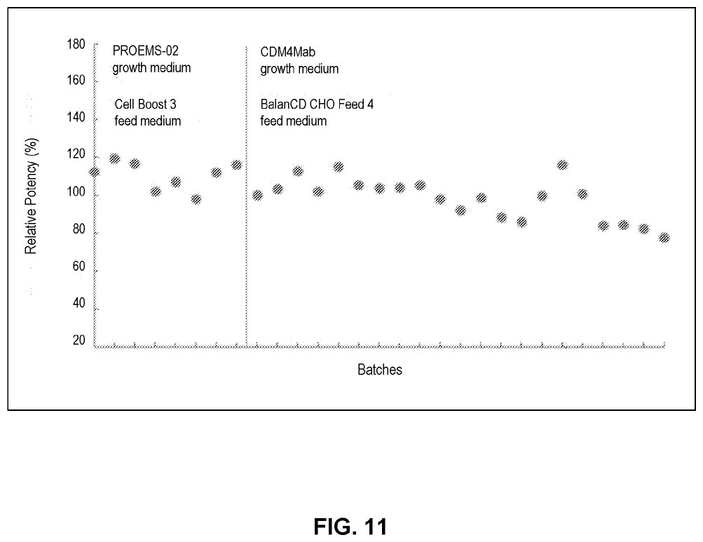

The cell culture growth and feed media identified in the aforementioned study was subsequently used in clinical GMP manufacturing of TG-1101, an anti-CD20 IgG1 monoclonal antibody. The drug substance material derived thereof was tested for % fucosylation as well as Fc effector function/activity. A comparison of the mAb characterization results produced using control media (PROEMS-02 growth medium and Cell Boost 3 feed medium) versus the new media (CDM4Mab® growth medium and BalanCD CHO Feed 4® feed medium) are shown in FIG. 6 for % fucosylation, FIG. 7 for FcγRIIIa-158V binding, FIG. 8 for CD20 binding, FIG. 9 for ADCC activity, FIG. 10 for C1q binding, and FIG. 11 for CDC activity. The results show a significant drop in % fucosylation, associated with a significant increase in FcγRIIIa-158V binding, and a modest increase in ADCC activity. There was no change in CD20 antigen binding as a result of switching the cell culture media. In addition, there was no change in C1q binding, and little to no change in CDC activity. Collectively, these results suggest that by changing the cell culture media for this rarely used YB2/0 rat hybridoma cell line, the function and activity of the expressed antibody can be modulated through cell culture media means alone. There have been no literature reports of this relationship in YB2/0 rat hybridoma cells in the public domain.

### Conclusion

In this Example, one way to increase process performance (cell growth, recombinant protein productivity) and modulate protein glycosylation from cell culture processes that employ YB2/0 cells was demonstrated. In this Example, cell growth and feed media were found to have a significant role towards protein glycosylation profiles. A growth and feed media combination was identified (CDM4Mab®, BalanCD CHO Feed 4®) that supported a significant decrease in percent fucosylation levels, which upon further testing, was found to support an increase in potency via FcγRIIIa binding.

### Example 2: Increasing Anti-CD20 Antibody Productivity Expressed by a YB2/0 Rat Hybridoma Cell Line Through Cell Culture pH, Temperature, and pCO2 Control Methodology

### Materials and Methods

### Cell Culture

A YB2/0 rat hybridoma cell line (ATCC CRL 1662) genetically engineered to stably express the anti-CD20 IgG1 monoclonal antibody, TG-1101, was thawed from cryopreservation and cultured in CDM4Mab (GE Healthcare) chemically-defined growth media at 37° C. After a series of passages into culture vessels of increasing volume, and after sufficient cell biomass was achieved, cells were inoculated into 10 L lab-scale bioreactors. Process conditions utilized during these cultures are shown in Table 6.

The 10 L bioreactors were equilibrated after cell growth media addition. Media was added to an initial volume target. Dissolved oxygen (DO) and pH probes were calibrated prior to use. Initial bioreactor set points included temperature, pH, dissolved oxygen, and agitation rate. The bioreactor was inoculated with cell culture from the seed train inoculum expansion vessels at a viable cell density target of 0.5×106 viable cells/mL. During the production bioreactor process, pH was controlled with base addition and CO2 sparging on demand. Dissolved oxygen was controlled with oxygen and air sparging on demand. Antifoam was added as needed to mitigate foaming concerns. Offline pH, pCO2/pO2, osmolality, and metabolites were monitored daily using a blood gas analyzer (Siemens) as were viable cell density (VCD) and viability using a ViCell automated cell counter (Beckman Coulter). Daily samples were also measured for process metabolites including glucose, lactate, glutamine, ammonia, as well as osmolality using a Nova Bioprofile FLEX2 analyzer (Nova Biomedical).

Throughout the course of the bioreactor cultures, various feed media and solutions were added to support the nutritional needs of the cells. BalanCD CHO Feed4® media (Irvine Scientific) was added on days 3, 5, 7, 9 at specified volumes (4.0% of initial bioreactor working volume). A calculated volume of concentrated glucose solution was fed (to 4.0 g/L) when daily bioreactor sample measured values were <3.0 g/L on specified process days. A fixed volume of glutamine solution was bolus fed on Day 3, and a calculated volume was fed (to 4.0 mM) when daily bioreactor sample measured values were <3.00 mM on specified process days. A 1000× concentrated cholesterol lipid solution was added to the cultures in fixed bolus additions on days 0 and 4 (0.256% v/v).

Throughout the course of the bioreactor cultures, process setpoints were shifted at fixed times. Culture pH setpoint was shifted from 7.10+/−0.30 to different pH setpoints (Table 6) on process day 3. Temperature setpoints were shifted from 37° C. to 35° C. on process day 1 and shifted again from 35° C. to 32.5° C. on process day 3.

The production bioreactor was harvested based on either culture duration or cell viability criteria, whichever occurred first. The cell culture harvest was clarified to remove the cells, and then protein A-purified for measurement of product quality (SEC, iCIEF, N-linked glycans) and functional testing, including both effector function and binding assays.

TABLE 6
Cell Culture Process Conditions Used
in Laboratory-Scale Bioreactors

| Process Parameter | Target/Range |
|---|---|
| Bioreactor volume (L) | 10 |
| Initial culture volume (L) | 6 |
| Initial temperature (° C.) | 37.0 |
| Initial viable cell density (×10⁶ cells/mL) | 0.50 |
| Initial cell viability (%) | ≥95.0 |
| Temperature after 1^{st} shift (° C.) | 35.0 |
| 1^{st} temperature shift timing (hrs) | 24 |
| Temperature after 2^{nd} shift (° C.) | 32.5 |
| 2^{nd} temperature shift timing (hrs) | 72 |
| pH at pre-inoculation | 7.40 |
| pH shift timing (hrs) | 72 |
| pH at post-inoculation | 7.10 +/− 0.10 |
| pH after shift | Various:^{a} |
| | 6.70 +/− 0.10 |
| | 6.80 +/− 0.03 |
| | 6.85 +/− 0.05 |
| | 6.95 +/− 0.05 |
| Agitation (rpm) | 264 |
| Dissolved oxygen (%) | 40.0 |
| BalanCD CHO Feed 4 addition amount | 4.0 |
| (% of initial working volume) | |
| BalanCD CHO Feed 4 addition timing (process days) | 3, 5, 7, 9 |
| Glutamine feed addition amount on Day 3 | 3.0 |
| (% of initial working volume) | |
| Glutamine concentration for triggering feeding (mM) | <3.00 |
| Glutamine feed addition timing (process days) | 4-7 |
| Glutamine concentration after feeding (mM) | 4.00 |
| Glucose concentration for triggering feeding (g/L) | <3.00 |
| Glucose feed addition timing (process days) | 3-11 |
| Glucose concentration after feeding (g/L) | 4.00 |
| Culture duration (days) | 14 |
| Harvest cell viability (%) | <20 |

^{a}Values reflect both the process setpoint +/− deadband values (i.e., the range centered on the process setpoint whereby the pH is free to move)

### Protein Analytical Characterization

### N-Glycans

N-linked glycans are cleaved from the product by enzymatic deglycosylation using PNGase F and labeled with 2-aminobenzamide (2-AB), a fluorescent compound. The labeled glycans are resolved using a hydrophilic interaction partition mode ultra-performance liquid chromatography (HILIC-UPLC) column equipped with a fluorescence detector (fluorescence excitation at 360 nm and emission at 428 nm). Blank, glycans from a reference standard, and glycan standards (human IgG N-linked glycan library) are also injected to assess system suitability. Peak identification from the resulting test sample chromatograms are identified based on retention time and relative to peaks in the glycan standard, which have been confirmed by mass spectrometry.

### SEC

The SEC-HPLC method is used to determine the molecular size distribution and purity of the IgG1 antibody. SE-HPLC separates proteins based on hydrodynamic radius and employs a TSKgel G3000SWXL column (Tosoh) designed to separate proteins in the range of 10,000 to 500,000 Da. The resulting chromatogram is monitored at 214 nm, the peaks are integrated, and the amount of each peak (monomer, dimer, aggregate, and fragments) is expressed as the percentage of its area. Samples, reference standard, molecular weight standards, and blank injections are performed to assess system suitability

iCIEF

Imaged Capillary Electrophoresis (iCIEF) is used to evaluate and quantify the distribution of charge-based isoforms of TG-1101 using a Protein Simple iCE3 Analyzer. The test sample solution includes 0.15% methylcellulose, 1.2 M urea, 8% ampholytes pH 3-10.5, and pH 10.10 and 8.40 pH markers. Focusing is conducted for one minute at 1500 V followed by 8 minutes at 3000 V. Reported results include the pI for the main peak, the % peak area for the main peak, as well as the pooled value for % peak area for the basic peaks and acidic peaks. A blank sample and hemoglobin control are also run to evaluate system suitability.

### ADCC Potency Assay

The ADCC assay is a cell-based assay using CD20 expressing Jeko-1 cells, a human mantle cell lymphoma cell line, and Eurofins-DiscoverX “KILR CD16a effector cells,” which are single donor-derived human CD8+ T-lymphocytes engineered to express CD16 (FcγRIII) on their plasma membrane surface. The target cell lysis mediated by antibody samples is measured. Dose response curves are modeled using 4PL equation, and results are reported as % potency relative to the reference standard.

### CDC Potency Assay

The CDC assay is a cell-based assay using the CD20 expressing Jeko-1 cells and rabbit serum as the source of complement. CDC-mediated cell lysis is measured. Dose response curve is modeled using 4PL equation, and results are reported as % potency relative to the reference standard.

### CD20 Binding Activity

In the CD20 binding activity assay, the binding of anti-CD20 antibody to the CD20 expressing human mantle cell lymphoma cell line, Jeko-1, is evaluated using the Meso Scale Discovery (MSD) method. Dose response curves are modeled using 4PL equation, and results are reported as % potency relative to the reference standard.

### FcγRIIIa-158V Binding:

The binding of anti-CD20 antibody samples to FcγRIIIa is evaluated by surface plasmon resonance (SPR). The method follows a direct binding assay methodology where the Fc receptor (ligand) is directly immobilized onto the appropriate flow cell on a sensor chip surface and the analyte is injected over the chip to assess binding. The high affinity allele of FcγRIIIa (158V) is determined.

The equilibrium dissociation constants (KD) and the relative affinities of each sample relative to the reference standard is determined for each receptor. The rates of change of the SPR signal is analyzed using a 1:1 Langmuir model to yield apparent rate constants for the association and dissociation phases of the reaction, and equilibrium dissociation constants. Results are reported as % potency relative to the reference standard for the FcγRIIIa-158V variant.

### C1q Binding:

The binding of anti-CD20 antibody to C1q is evaluated using a C1q ELISA-based method. The dose-response binding is modeled by weighted nonlinear regression using a 4-parameter logistic fit, and the results are reported as % potency relative to the reference standard.

### Results

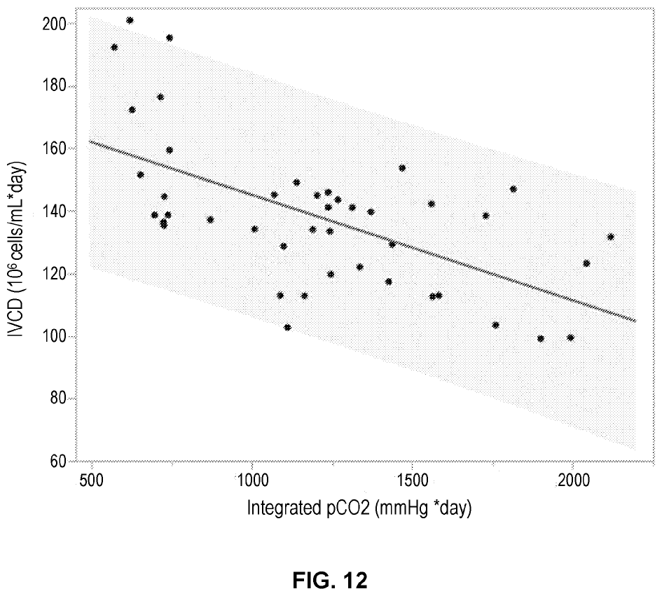

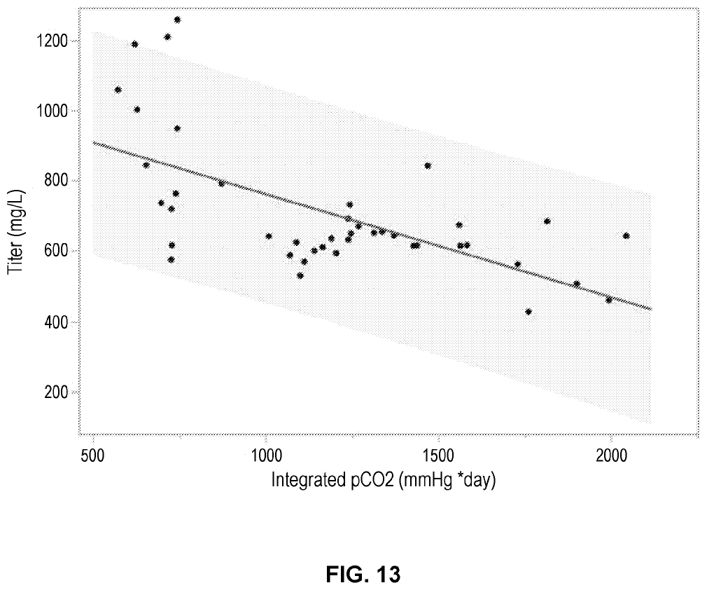

Retrospective analysis of manufacturing and laboratory scale cell cultures of the YB2/0 rat hybridoma cell line was performed to analyze for trends in the process performance data. One of the most significant trends in the data was the positive relationship between cell growth and cumulative culture exposure to pCO2 (FIG. 12). In general, cultures with high cumulative cell growth (IVCD) also had lower cumulative exposure to pCO2 (integrated pCO2). Another significant trend in the data was the positive relationship between harvest titer and cumulative culture exposure to pCO2 (FIG. 13). In general, cultures with high harvest titer of the monoclonal antibody also had lower integrated pCO2. A further investigation of these relationships was pursued. 10 L laboratory scale cultures were evaluated specifically testing the use of different pH control strategies to facilitate changes in pCO2.

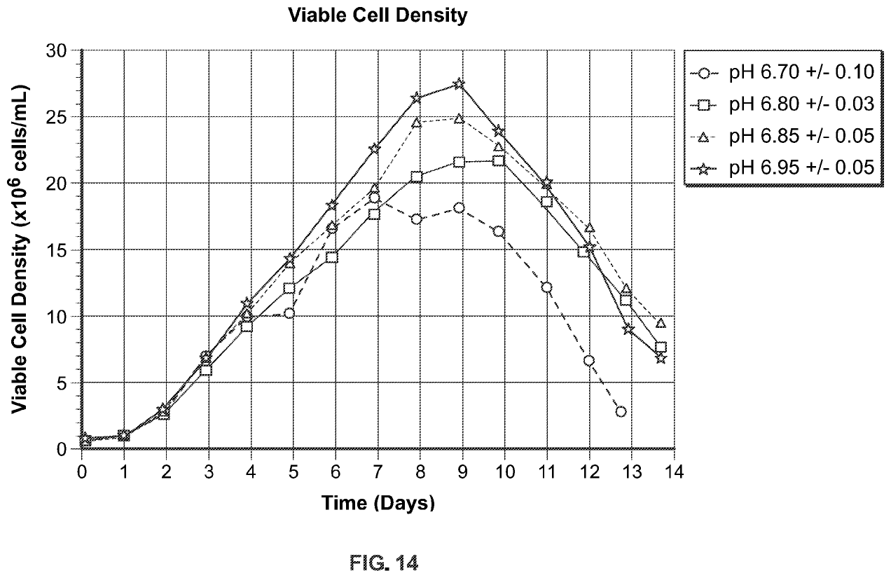

Production bioreactor cultures at the laboratory-scale were performed evaluating different post-shift pH setpoints and control ranges. Cell growth results are shown in FIG. 14 and demonstrate a pH-dependent impact on the results observed. At the lowest post-shift, pH control range studied (6.70+/−0.10), peak viable cell density (VCD) was the lowest amongst the tested conditions (18.9×106 cells/mL). In contrast, at the highest pH control range studied (6.95+/−0.05), peak VCD was the highest amongst the tested conditions. pH control ranges in between these two ranges demonstrated cell growth and peak VCD results that were intermediate in nature. The results highlight that as the process pH control range increases, there is a significant increase in cell growth observed. Lower cell growth with lower culture pH has been seen in CHO cells before (Trummer, E. et al., Biotechnology and Bioengineering 94:1033-1044 (2006)). However, this behavior has never been reported with a YB2/0 cell line.

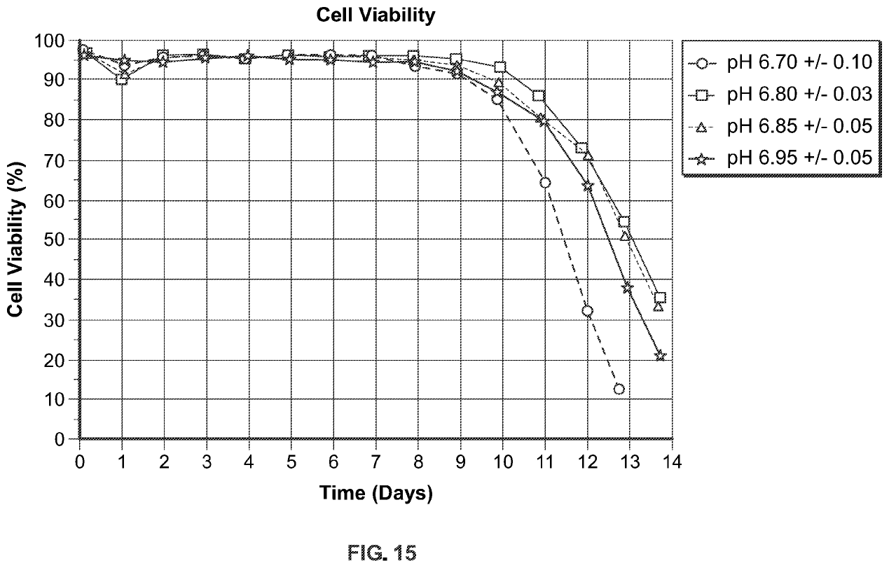

Cell viability results also showed a trend at the different pH control ranges and are shown in FIG. 15. At the lowest pH control range studied, cell viability dropped earlier, triggering a bioreactor harvest on process day 13. At the higher pH control ranges studied, cell viability dropped a lot slower, leading to a bioreactor harvest on process day 14. These results are significant because it suggests that controlling pH alone for this unique cell line is a process means with which culture longevity can be increased. Along with this prolonged culture longevity, the resulting impact to harvest titer is also significant.

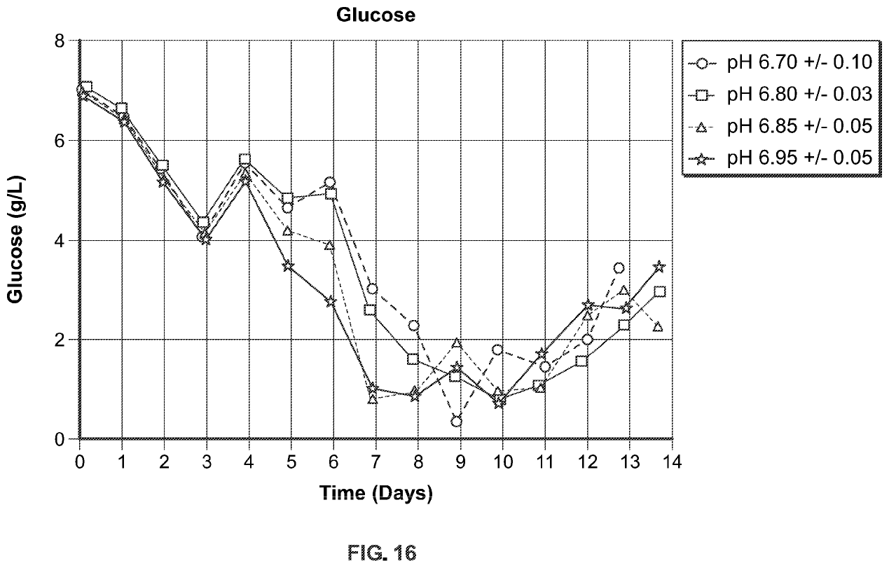

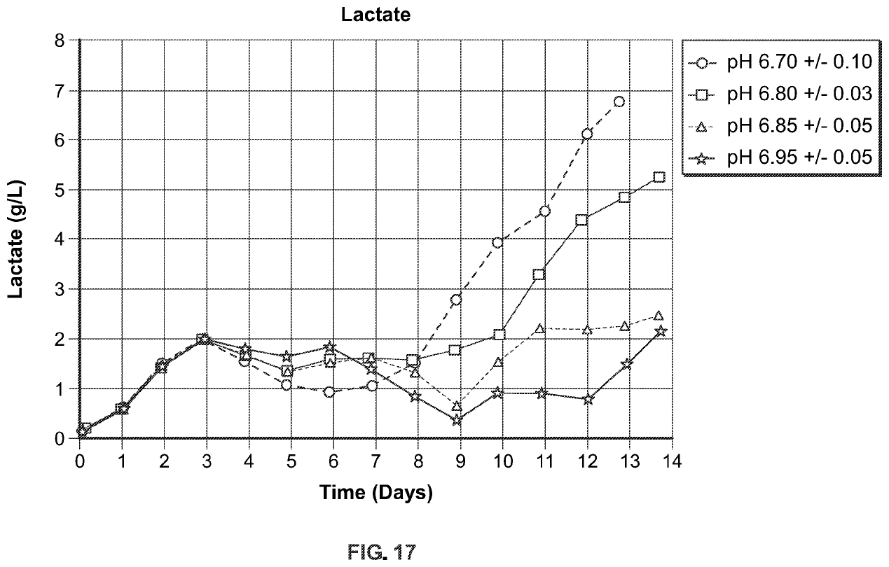

Glucose is consumed by mammalian cells as the primary source of energy and carbon necessary for carrying out oxidative metabolism. A principal waste byproduct from glucose metabolism in mammalian cells is lactate. High levels of lactate have previously been shown to be inhibitory towards process performance (Hassell, T. et al., Appl Biochem Biotechnol. 30: 29-41 (1991)). Glucose and lactate levels in the cell culture media were monitored throughout each of the cultures (FIG. 16, FIG. 17). Glucose levels were similar throughout each of the cultures and did not deplete since a concentrated glucose feed solution was added into each of the cultures as needed. There were however major differences in lactate levels observed from Day 9 through harvest. The lower pH process conditions supported higher lactate levels, with the pH 6.70+/−0.10 condition supporting the highest final lactate level of 6.8 g/L, and the pH 6.95+/−0.05 condition supporting the lowest final lactate level of 2.2 g/L. These results suggest that there are major differences in metabolism as a function of process pH control for YB2/0 cells, and that this is the first time this relationship is being reported for this unique cell line.

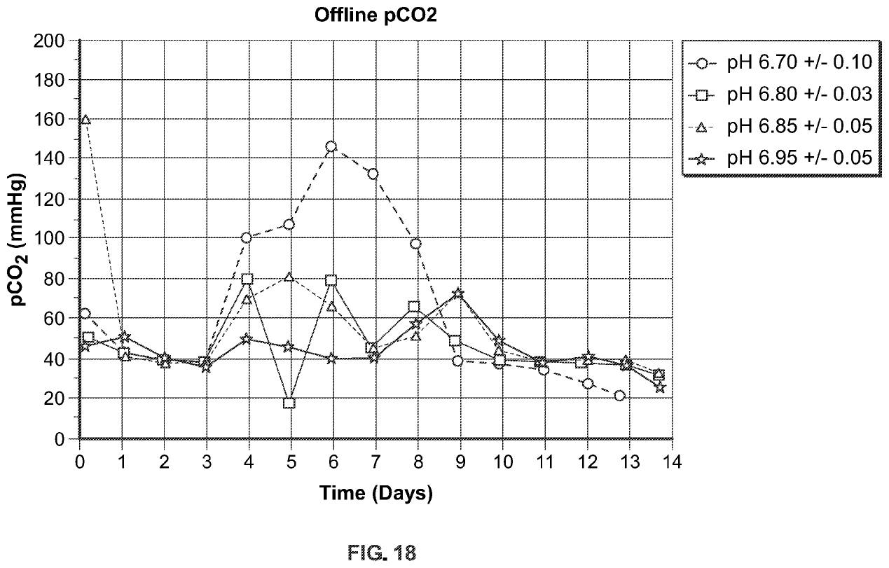

Dissolved carbon dioxide levels were also monitored throughout each of the respective cell culture conditions (FIG. 18). High levels of pCO2 have been shown previously with other mammalian cell lines to adversely impact overall process performance (deZengotita, V., et al., Cytotechnology 28: 213-227 (1998)). The in-process pCO2 levels observed during bioreactor conditions is a function of both the pH setpoint, the levels of lactate in culture (with higher levels of lactate supporting a more acidic environment), as well as the overall air and oxygen sparge rates in the bioreactor. In each of the 10 L bioreactor cultures evaluated, the same maximum air sparge rates were used. Oxygen sparge rates were controlled by the bioreactor controller, with higher flowrates typically required for cultures with higher VCD levels. The YB2/0 cell culture results show that the lowest pH control condition supported the highest levels of pCO2 observed. That is, the pH 6.70+/−0.10 culture led to a peak pCO2 level of 146 mmHg in the middle of the culture, whereas the higher pH control conditions all supported pCO2 levels <100 mmHg. Collectively, these results highlight how the higher pH control conditions supported the lowest pCO2 levels, as well as the highest cell growth profiles, and antibody titers as discussed below.

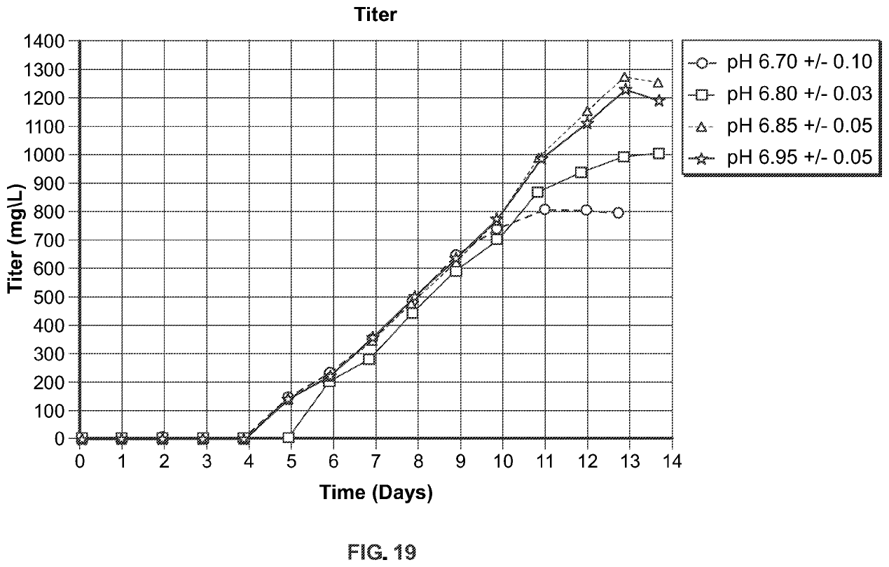

Antibody titers were monitored throughout each of the aforementioned laboratory-scale cell culture conditions (FIG. 19). In all pH control ranges evaluated, the antibody titers started increasing between process days 4-5. The antibody titers similarly increased to approximately the same levels up until day 10, after which they began to diverge. The higher pH culture conditions continued to increase up until the point of harvest, where harvest day titers measured were 1.26 g/L for pH condition 6.85+1-0.05, and 1.19 g/L for pH condition 6.95+/−0.05 g/L. The lower pH culture conditions did not demonstrate as large of a titer increase after day 10, leading to harvest titers of 1.0 g/L for pH condition 6.80+/−0.03 and 0.79 g/L for pH condition 6.70+/−0.10. The collective results show that as the post-shift pH control range increases to a higher value, pCO2 decreases, and similar to the behavior highlighted in FIG. 13, the resulting antibody productivity increases.

These results are surprising since it has been previously found in other cells lines that an increase in culture pH supports a decrease in productivity (Jiang, R. et al., Bioprocess Biosyst Eng 41: 1731-1741 (2018)), and a decrease in culture pH supports an increase in productivity (Seo, J. S. et al., Appl Microbiol Biotechnol. 97: 5283-5291 (2013)). In other reports with CHO cell lines, the results have been mixed. In two reports, an increase in recombinant productivity was seen at higher pH values, but in the pH range of 7.00-7.20 (Yoon, S. K. et al., Biotechnol. Bioeng. 89:345-356 (2005); Kim, H. S. et al., J Microbiol and Biotech. 17:712-720 (2007)). In another report, culture pH was found to have no impact on productivity (Hennicke, J. et al., New Biotechnology 50:20-26 (2019)). In a different report with a CHO cell line expressing an antibody, pH 6.8 was found to support a 2× higher titer compared to pH 7. (Oguchi, S. et al., Animal Cell Technology; Basic & Applied Aspects 13:169-172 (2003)). It is believed that there are no available reports describing the relationship of culture pH on YB2/0 cells, and the present work is believed to be the first documented instance.

The expressed antibody (TG-1101) was protein A-purified from each of the 10 L bioreactor cultures and analyzed for product quality, including N-linked glycans (protein glycosylation), charge heterogeneity (iCIEF), and size (SEC). Table 7 summarizes the N-linked glycans and highlights how the highest pH control range (6.95+/−0.05) supported the lowest level of % fucosylation (18.4%). In addition, as the pH control range increased (i.e., 6.80+/−0.03 and higher), the total amount of sialylated N-glycans increased from 0/a to 1%, effectively introducing a new N-glycan species into the profile. Protein fucosylation levels can thus be nominally controlled through pH mediated pCO2 control for this unique YB2/0 cell line. Table 8 summarizes the charge heterogeneity results, which were not significantly impacted through the different pH and pCO2 conditions. Table 9 summarizes the size characterization results of the antibody, and highlights no change in individual species across the different pH and pCO2 conditions.

TABLE 7
Protein glycosylation results from cultures evaluating
different pH control ranges (post-shift)

| pH Control | Fucosylation | Sialylation | | | |
|---|---|---|---|---|---|
| Range | (%) | (%) | G0 (%) | G0B (%) | G1 (%) |
| 6.70 +/− 0.10 | 24.6 | 0 | 50.7 | 8.9 | 9.7 |
| 6.80 +/− 0.03 | 27.5 | 0.8 | 47.1 | 7.8 | 8.3 |
| 6.85 +/− 0.05 | 24.9 | 0.8 | 45.9 | 6.5 | 11.5 |
| 6.95 +/− 0.05 | 18.4 | 0.3 | 46.7 | 7.0 | 11.8 |

TABLE 8
iCIEF results from cultures evaluating different
pH control ranges (post-shift)

| pH Control Range | Acidic Species (%) | Main (%) | Basic Species (%) |
|---|---|---|---|
| 6.70 +/− 0.10 | 22.8 | 56.9 | 20.4 |
| 6.80 +/− 0.03 | 22.9 | 59.3 | 18.0 |
| 6.85 +/− 0.05 | 25.2 | 58.5 | 16.3 |
| 6.95 +/− 0.05 | 28.2 | 52.8 | 19.1 |

TABLE 9
SEC Results from cultures evaluating different
pH control ranges (post-shift)

| | Monomer | Dimer | Aggregates | Fragments |
|---|---|---|---|---|
| pH Control Range | (%) | (%) | (%) | (%) |
| 6.70 +/− 0.10 | 98.9 | 1.1 | 0 | 0 |
| 6.80 +/− 0.03 | 98.9 | 1.1 | 0 | 0 |
| 6.85 +/− 0.05 | 98.3 | 1.6 | 0 | 0.2 |
| 6.95 +/− 0.05 | 98.4 | 1.3 | 0 | 0.3 |

Purified antibody (TG-1101) from each of the 10 L bioreactor cultures was also tested in various binding assays (CD20 antigen, Fc gamma receptor, complement component 1q), as well as Fc effector activity assays (ADCC, CDC). The results from each of these tests are shown in Table 10. FcγRIIIa-158V, CD20, and C1q binding results were comparable across each of the culture conditions evaluated. ADCC and CDC bioassay results were also shown to be comparable across each of the culture conditions. Collectively, these results highlight that the higher cell growth and antibody titers elicited through the higher process pH control and associated lower pCO₂ control did not adversely impact the functional activity of the antibody.

TABLE 10
Binding/effector function testing results from cultures
evaluating different pH control ranges (post-shift)

| | FcγRIIIa- | CD20 | ADCC | CDC | C1q |
|---|---|---|---|---|---|
| pH Control | 158V | binding | activity | activity | binding |
| Range | binding (%)^{a} | (%)^{a} | (%)^{a} | (%)^{a} | (%)^{a} |
| 6.70 +/− 0.10 | 119.9 | 115.6 | 94.5 | 98.6 | 86.6 |
| 6.80 +/− 0.03 | 98.2 | 96.8 | 91.3 | 87.4 | 84.5 |
| 6.85 +/− 0.05 | 108.3 | 89.5 | 108 | 102 | 81.2 |
| 6.95 +/− 0.05 | 108.7 | 109.5 | 93.1 | 90.9 | 76.5 |

^{a}Results expressed relative to the reference standard used for each assay.

### CONCLUSION

In this example, the inventors demonstrated another way to increase process performance (cell growth, recombinant protein productivity), and how to modulate protein glycosylation from cell culture processes that employ YB2/0 cell lines. In this example, the process parameter of cumulative pCO₂ exposure during cell culture was found to have a significant role towards both cell growth and antibody productivity. Culture pH was confirmed to be a primary driver towards culture pCO₂ levels. Both a higher pH control range (>6.80), as well as control of low pCO₂ levels (<100 mmHg) in culture were found to be supportive of a high process titer (≥1.0 g/L) for this unique, uncommonly used mammalian expression system. In the bioreactor high productivity state, the successful demonstration of the control of % fucosylation levels was shown, as well as the resulting binding and effector function/activity of the expressed anti-CD20 antibody.

### Example 3: Commercial-Scale Manufacturing Process of Making TG-1101 in YB2/0 Rat Hybridoma Cells

In this Example, the manufacturing process for TG-1101 (TG Therapeutics, Inc.) expressed in YB2/0 rat hybridoma cells at 15,000 L is described. An overview of the manufacturing process for TG-1101 is illustrated in the flow diagram of Table 12.

TABLE 12
TG-1101 Manufacturing Process Flow Diagram
Vial of TG-1101 Working Cell Bank
↓
Inoculum Expansion
(Shake flasks, 50 L cellbag)
↓
Seed Bioreactors
(120 L, 600 L, 3000 L)
↓
Production Bioreactor
(Fed-batch 15,000 L)
↓
Harvest
(Disc centrifuge)
(A1HC Pod depth filter, 1.2/0.5 μm filter, 0.45/0.2 μm filter)
↓
Capture Column Chromatography
(MabSelect SuRe Protein A)
↓
Solvent/Detergent Viral Inactivation (SDVI)
(TnBP/Polysorbate 80)
↓
Cation Exchange Chromatography (CEX)
(SP Sepharose)
↓
Anion Exchange Membrane Chromatography (AEX)
(Mustang Q)
↓
Viral Filtration (VF)
(Viresolve ®)
↓
Ultrafiltration/Diafiltration (UFDF)
(30 kDa NMWCO)
↓
Formulation, Filtration
(Polysorbate 80 addition; 0.2 μm filtration)
↓
Fill and Store
(Freeze at ≤−60, store at ≤−35° C.)

In summary, the production of each batch of TG-1101 began with the thaw of a working cell back (WCB) vial, described further below. The culture was expanded through a series of shake flasks and seed bioreactors to meet the inoculum requirements of the 15,000 L production bioreactor, which was operated in fed-batch mode. The bioreactor was harvested and clarified by centrifugation followed by depth filtration. The clarified harvest was purified by three chromatography steps including Protein A, cation exchange, and anion exchange, designed to purify the TG-1101 and to reduce process impurities such as host cell protein and residual DNA. The purification process (as illustrated in Example 4) contained steps to ensure viral safety including viral inactivation (solvent/detergent) and a viral filtration step. Final UFDF and formulation steps are used to concentrate and buffer exchange TG-1101 into the formulation buffer and to the desired product concentration. The ready to fill drug substance was formulated to obtain TG-1101 at a concentration of 25.0 mg/mL in 25 mM sodium citrate, 154 mM sodium chloride, 0.07% polysorbate 80, pH 6.5. After filling, the TG-1101 drug substance (DS) was frozen at ≤−60° C. and then stored frozen at ≤−35° C.

Additional descriptions of the upstream and downstream process operations are provided below.

### Expression Vector, Production Cell Line, and Cell Banks

The host cell line used for generation of the TG-1101 producing cell line was the rat hybridoma cell line YB2/0. The production cell line, R603-12D11, was developed after transfecting the expression vector HK463-25 (containing the immunoglobulin heavy and light chain cDNA sequences of TG-1101) into the YB2/0 host cell line. FIG. 20 depicts the expression vector map of HK463-25 to produce TG-1101, in a 15,000 L bioreactor.

### Expression Vector

The expression vector HK463-25 included various elements that were optimized for stable expression in the YB2/0 host cell line. The Rous Sarcoma Virus Long Terminal Repeat (RSV LTR) promoter was used for the constitutive expression of both heavy and light chain cDNAs. This promoter corresponds to the Long Terminal Repeat of the RSV genome which contains enhancer elements in its 5′ region and has strong transcriptional activity in the YB2/0 cell line. Transcriptional termination and polyadenylation of both heavy and light chain cDNAs were provided by the human growth hormone polyadenylation sequence (hGH polyA). A chimeric intron was introduced 5′ to the cDNA sequence of each antibody chain to improve expression. This intron was optimized for splicing and is composed of a 5′ donor sequence from human beta-globin and a 3′ acceptor sequence from an Ig heavy chain variable gene. The beta lactamase gene conferred ampicillin resistance (AmpR) and was provided to enable production of the plasmid in E. coli. The enzyme neomycin-phosphotransferase II (NeoR) was under the control of the SV40 promoter and confers resistance to the transfected cell line to the antibiotic G418, thus acting as a selectable marker. Dihydrofolate reductase (Dhfr) was under the control of the SV40 promoter and conferred resistance to methotrexate (MTX) and can also act as a selectable and amplifiable marker in the transfected cell line.

The HK463-25 expression vector (FIG. 20) was 11.1 kb in size and contained five open reading frames for the antibody heavy chain, light chain, Dhfr, NeoR and AmpR genes in the same orientation. The restriction sites shown in the figure were used for Southern blot analysis of the integration of the construct. A unique NotI restriction site located 3′ to the NeoR gene was used for linearization of the vector prior to transfection.

### Production Cell Line, R603-12D11

The production cell line, R603-12D11, was generated after transfection of the host cell line, selection and screening of transfectants, and then limiting dilution cloning. The clones were screened, production cell line R603-12D11 was selected, and adapted to serum free medium. A pre-seed stock (PSS) cell bank was prepared. An overview of the steps involved in the generation of production cell line R603-12D11 is shown in Table 13.

TABLE 13
Production Cell Line R603-12D11 Generation

| | |
|---|---|
| Host Cell line | Linearized expression |
| YB2/0 from ATCC | vector HK463-25 |
| ↓ | ↓ |

Transfection of the YB2/0 Host Cell Stock with the
HK463-25 expression plasmid
↓
Selection of transfectants with G418
↓
Screening of transfectants to identify candidate cell lines
↓
Cloning by limiting dilution at 0.4 cell/well
↓
Screening of clones leading to the selection of cell line R603-12D11
↓
Expansion and adaptation of R603-12D11 to serum free media
↓
Expansion and preparation of a pre-seed stock (PSS)/cell
bank for R603-12D11 (serum free)

A cryovial of the YB2/0 cell bank (YB2/0-301 04/147) was thawed and the cells were grown by dilution to a cell density of 1×10⁵ cells/mL every 3 to 4 days with fresh culture medium (EMS medium with 5% FCS). The cells were seeded at a density of 2×10⁵ cells/mL on the day before transfection in order to reach exponential phase prior to transfection. 44.5 μg of the NotI-linearized expression vector HK463-25 (FIG. 20) was transfected into 5×10⁶ cells by electroporation using the animal component free Optimix reagent (Equibio). The cells were diluted in culture medium and seeded at 100 cells/well in 96-well plates. Selection with 1 g/L G418 in the culture medium was initiated three days after electroporation.

The transformants were initially screened for titer by ELISA after selection in the G418 medium. Over 3000 transformants were screened and over 200 of the best producing wells were selected for further testing and for continued passaging. A second titer screen was performed to further reduce the number of clones. This screen was followed by additional screening for antibody fucose level by ELISA. Fucose levels less than 40% were considered desirable in order to provide the expected level of activation of CD16 by the antibody; CD16 activation is inversely related to the fucose levels. Further screening using a cell-based CD16 activation test with assessment of IL-2 secretion in addition to assessment of the free Kappa/IgG ratio (<0.2 for purification ease) resulted in narrowing to a total of 5 cell lines for further development.

The five candidate cell lines were cloned by limiting dilution at 0.4 cells/well in EMS medium with 5% FCS. Clones were screened for IgG productivity, fucose levels, CD16 activation, and free Kappa/IgG ratio to identify production cell line R603-12D11. As the selected cell line was expanded from the cloning step, it also underwent a transition into serum free media and was re-screened to ensure the desired phenotype before the preparation of a small cell bank. This small cell bank was then thawed and expanded to generate the R603-12D11 pre-seed stock cell bank (PSS). The R603-12D11 PSS cell bank was demonstrated to be free from mycoplasmal, adventitious virus and microbial contamination prior to the generation of the MCB.

Characterization of the master and working cell banks derived from the R603-12D11 production cell line, included testing for identity, genotypic and phenotypic features, as well as for the presence of adventitious agents.

### Master Cell Bank (MCP) Preparation and Testing

The MCB was manufactured at Henogen (later acquired by NovaSep). The MCB lot G071/MCB/070208 was prepared by thawing and expanding one vial of the production cell line R603-12D11 pre-seed stock in serum-free medium EM-SF2 P500 H4 (EMS basal medium supplemented with 2-mercaptoethanol, ethanolamine, NaHCO₃, ferric citrate, pluronic acid, HEPES and recombinant human insulin). The cells were expanded in T-flasks and roller bottles for eleven days. The cell suspension was concentrated by centrifugation and aliquoted into 13 separate fractions. Each fraction was centrifuged and resuspended to a target cell density of 10×10⁶ cell/mL in freezing medium (90% EM-SF2 P500 H4+10% DMSO). The suspended fractions were then each aliquoted into 18 cryovials per fraction resulting in a total of 234 cryovials of the MCB. The cryovials were placed on dry ice and then into cryoboxes which were placed in a −80° C. freezer for 21 hr. The cryovials were transferred into liquid nitrogen tanks on 19 Feb. 2007 for long-term storage and are currently stored in multiple locations. The number of cell generations from the PSS of the production cell line to the MCB is 10.4.

Following the generation of MCB lot G071/MCB/070208, direct testing of the MCB, as well as further characterization by testing a WCB derived from it, were performed. Identity testing of the MCB was performed; testing results confirm the identity of the MCB as rat derived. Performance qualification was confirmed by thawing a vial of the MCB and monitoring for cell viability. Assessments of copy number, restriction endonuclease profile, number of integration sites, integrity of RNA coding sequence, and RNA quantitation were conducted. The number of heavy and light chain copies integrated into the genome was estimated at 1.13 and 2.14, respectively, by quantitative polymerase chain reaction (Q-PCR). Southern blot assessment of the plasmid integration site demonstrated comparable hybridization patterns for the MCB and PSS. The number of integration sites for both the MCB and WCB was similarly measured to be 1 in both cell banks via Southern blot. In addition, intra-chromosomic integration of the HK463-25 expression plasmid with insertion at a single locus of a metacentric chromosome was demonstrated by fluorescence in situ hybridization (FISH) analysis. For the MCB, integrity of the RNA coding sequence was consistent with the reference sequence, and RNA quantitation was consistent with the PSS cell bank for both heavy and light chains.

Next generation nucleic acid sequencing (NGS) using targeted locus amplification (TLA) was performed (Cergentis, Utrecht, Netherlands) on the MCB to confirm that the expressed TG-1101 antibody has the correct amino acid sequence, and to also interrogate for low levels of sequence variants. Adventitious viral, mycoplasmal and microbial agent testing of the MCB was carried out and acceptance criteria established for release of the MCB including those for adventitious viral, mycoplasmal and microbial agents were met. The collective MCB testing results confirmed the suitability for the establishment of the MCB.

### Working Cell Bank Lot G140/R603/WCB001

The first WCB was manufactured at NovaSep (after acquiring Henogen). To prepare the WCB, one vial of MCB G071/MCB/070208 was thawed and expanded in serum-free medium EM-SF2 P500 H4 in flasks and roller bottles over eleven days. The expanded cell suspension was concentrated by centrifugation and aliquoted into 22 identical fractions. Each fraction was centrifuged and resuspended to a target cell density of 12.1×10⁶ cell/mL in freezing medium (90% EM-SF2 P500 H4+10% DMSO). The suspended fractions were then each aliquoted into 18 cryovials resulting in a total of 396 cryovials of the WCB. The lot was designated as G140/R603/WCB001. The cryovials were placed on dry ice and then into cryoboxes which were placed in a −80° C. freezer for 24 hr. The cryovials were transferred into liquid nitrogen tanks for long-term storage on 22 Sep. 2009. The WCB is stored in at least two different storage sites including smaller numbers of vials stored for shorter duration at the manufacturer. The number of cell generations from the PSS of the production cell line to the WCB is 21.4.

Testing of WCB lot G140/R603/WCB001 was performed including identity testing. Testing results confirmed the identity of the WCB as rat derived. Performance qualification was confirmed by thawing a vial of the WCB and monitoring for cell viability, doubling time, and IgG productivity. Assessments of copy number, restriction endonuclease profile, and number of integration sites, were conducted. The number of heavy and light chain copies integrated into the genome was estimated at 1.2 and 2.5, respectively, by quantitative polymerase chain reaction (Q-PCR). These results are consistent with that of the MCB. Southern blot assessment of the plasmid integration site demonstrated comparable hybridization patterns for the WCB and MCB. The number of integration sites for the WCB was measured to be 1 via Southern blot.

Next generation nucleic acid sequencing (NGS) using targeted locus amplification (TLA) was performed (Cergentis, Utrecht, Netherlands) on the WCB to confirm that the expressed TG-1101 antibody has the correct amino acid sequence, and to also interrogate for low levels of sequence variants. Adventitious viral, mycoplasmal, and microbial agent testing was conducted and all acceptance criteria established for release of the WCB including those for adventitious viral, mycoplasmal and microbial agents were met. The collective WCB testing results confirmed the suitability for the establishment of the WCB.

### Phenotypic Characterization of Cell Banks

Cell line phenotypic stability studies were performed for the MCB and two WCBs generated for manufacturing of TG-1101. One vial each of the MCB, WCB lot G140/R603/WCB001, and a new WCB lot 127646-001 were thawed and passaged for approximately 60 generations. VCD, cell viability, and titer samples were taken at every passage. At intervals of approximately 15 cell generations, cells were cryopreserved as research cell banks (RCBs). After approximately 60 generations, vials from each RCB were thawed and expanded for 7 days. On day 7, shake flasks were inoculated, cultured under fed-batch conditions, and cultivated for 12 days. Daily sampling for VCD, cell viability, titer, specific productivity, and quality attributes of the harvested fed batch culture were used to assess the stability of each cell bank at its corresponding generation number. Comparability in cell growth, titer, and product quality was used as a general basis for determining phenotypic stability of the cell banks. In addition, specific productivity results that exceed 70% of the results from control cultures with low number of generations were used as a specific basis for determining phenotypic stability of the cell banks. The results for the three cell bank phenotypic stability studies are shown in Table 14 for MCB lot G071/MCB/070208, Table 15 for WCB lot G140/R603/WCB001, and Table 16 for WCB lot 127646.

TABLE 14
Phenotypic Stability Results of MCB Lot G071/MCB/070208

Number of Generations^{a}

| Parameter | 9 | 28 | 40 | 54 | 71 |
|---|---|---|---|---|---|
| Peak viable | 12.6 | 11.7 | 12.4 | 11.9 | 11.9 |
| cell density | | | | | |
| (×10⁶ cells/mL) | | | | | |
| Harvest titer (mg/L) | 681 | 703 | 718 | 704 | 679 |
| Average specific | 6.6 | 6.7 | 6.8 | 6.8 | 6.6 |
| productivity | | | | | |
| (pg/cell/day) | | | | | |
| Glycosylation: | | | | | |
| % fucosylation | 32.8 | 34.1 | 37.4 | 36.1 | 37.1 |
| iCIEF: | | | | | |
| % acidic species | 26.0 | 27.2 | 27.8 | 27.7 | 28.9 |
| % main species | 38.1 | 47.5 | 46.4 | 46.4 | 48.4 |
| % basic species | 35.9 | 25.3 | 25.8 | 25.9 | 22.7 |
| SEC: | | | | | |
| % monomer | 97.7 | 97.6 | 97.5 | 97.5 | 97.3 |
| % dimer | 1.6 | 1.7 | 1.8 | 1.8 | 2.0 |
| % aggregates | 2.3 | 2.4 | 2.4 | 2.5 | 2.7 |
| % fragments | 0.1 | 0.1 | 0.1 | 0.1 | 0.1 |

^{a}After MCB vial thaw to the start of production fedbatch culture

TABLE 15
Phenotypic Stability Results of WCB Lot G140/R603/WCB001

Number of Generations^{a}

| Parameter | 9 | 26 | 40 | 54 | 71 |
|---|---|---|---|---|---|
| Peak viable | 15.6 | 17.0 | 14.7 | 15.6 | 16.1 |
| cell density | | | | | |
| (×10⁶ cells/mL) | | | | | |
| Harvest titer | 730 | 775 | 631 | 584 | 646 |
| (mg/L) | | | | | |
| Average specific | 5.2 | 5.6 | 5.5 | 5.1 | 4.8 |
| productivity | | | | | |
| (pg/cell/day) | | | | | |
| Glycosylation: | | | | | |
| % fucosylation | 37.4 | 39.7 | 39.2 | 38.7 | 39.7 |
| iCIEF: | | | | | |
| % acidic species | 22.7 | 25.6 | 30.0 | 27.4 | 29.7 |
| % main species | 52.6 | 44.2 | 42.9 | 45.8 | 44.9 |
| % basic species | 24.7 | 30.2 | 27.2 | 26.8 | 25.5 |
| SEC: | | | | | |
| % monomer | 98.1 | 97.7 | 97.4 | 97.7 | 97.6 |
| % dimer | 1.3 | 1.7 | 1.8 | 1.8 | 1.8 |
| % aggregates | 1.8 | 2.2 | 2.5 | 2.2 | 2.4 |
| % fragments | 0.1 | 0.1 | 0.1 | 0.1 | 0.1 |

^{a}After WCB vial thaw to the start of production fedbatch culture

TABLE 16
Phenotypic Stability Results of WCB Lot 127646

Number of Generations^{a}

| Parameter | 9 | 27 | 42 | 55 | 69 |
|---|---|---|---|---|---|
| Peak viable | 14.6 | 15.3 | 15.3 | 14.0 | 14.6 |
| cell density | | | | | |
| (×10⁶ cells/mL) | | | | | |
| Harvest titer | 770.5 | 865.5 | 801.3 | 886.2 | 849.7 |
| (mg/L) | | | | | |
| Average specific | 6.2 | 6.2 | 6.1 | 6.4 | 6.0 |
| productivity | | | | | |
| (pg/cell/day) | | | | | |
| Glycosylation: | | | | | |
| % fucosylation | 30.9 | 30.2 | 33.2 | 36.6 | 37.6 |
| iCIEF: | | | | | |
| % acidic species | 23.8 | 25.8 | 25.8 | 28.2 | 28.5 |
| % main species | 40.1 | 50.7 | 42.8 | 34.0 | 36.7 |
| % basic species | 36.1 | 23.5 | 31.4 | 37.8 | 34.8 |
| SEC: | | | | | |
| % monomer | 98.2 | 98.2 | 98.4 | 98.0 | 97.6 |
| % dimer | 1.3 | 1.3 | 1.2 | 1.4 | 1.8 |
| % aggregates | 1.8 | 1.7 | 1.6 | 1.9 | 2.4 |
| % fragments | 0.1 | 0.1 | 0.1 | 0.1 | 0.1 |

^{a}After WCB vial thaw to the start of production fedbatch culture

Thawed MCB and WCB that were use during production of TG-1101 at 15,000 L were tested for viable cell density and cell viability results to assess and confirm cell bank storage stability.

### Upstream Process and Process Controls

A process flow diagram of the upstream unit operations including operational controls and in-process controls are provided in Table 17. Among other controls, bioburden and endotoxin were measured in the batched media of the seed and production bioreactor stages.

TABLE 17
Upstream Operational and In-Process Controls

| Operational | | In-Process |
|---|---|---|
| Controls | Unit Operation | Controls |
| Temperature | Inoculum Expansion | Initial VCD |
| CO₂ | 125 mL shake flask (Stage 1) | Final VCD |
| | 500 mL shake flask (Stage 2) | Final cell viability |
| | 3 L shake flask (Stage 3) | Culture duration |
| | 3 × 3 L shake flasks (Stage 4) | Bioburden |
| | 50 L cellbag (Stage 5) | Endotoxin |
| | ↓ | |
| Temperature | Seed Bioreactor 1 | Initial VCD |
| DO | 120 L stainless steel | Final VCD |
| pH | bioreactor | Final cell viability |
| | | Culture duration |
| | | Bioburden |
| | | Endotoxin |
| | ↓ | |
| Temperature | Seed Bioreactor 2 | Initial VCD |
| DO | 600 L stainless steel | Final VCD |
| pH | bioreactor | Final cell viability |
| | | Culture duration |
| | | Bioburden |
| | | Endotoxin |
| | ↓ | |
| Temperature | Seed Bioreactor 3 | Initial VCD |
| DO | 3000 L stainless steel | Final VCD |
| pH | bioreactor | Final cell viability |
| | | Culture duration |
| | | Bioburden |
| | | Endotoxin |
| | ↓ | |
| Temperature | Production Bioreactor | Initial VCD |
| Temperature | 15000 L stainless steel | Initial cell viability |
| shift timing | bioreactor | Final cell viability |
| DO | | Culture duration |
| pH | | Bioburden |
| pH shift timing | | Endotoxin |
| Feed media addition | | |
| Glutamine feed | | |
| addition | | |
| Glucose feed | | |
| addition | | |

### Cell Culture Media and Feeds Preparation

Cell culture growth medium (CDM4Mab) and feeds (BalanCD CHO Feed4, glucose feed, and glutamine feed) were prepared with Water for Injection (WFI). Media and feeds were filtered (≤0.2 μm) into sterile vessels and stored prior to use as needed. A cholesterol lipid concentrate was supplemented into the CDM4Mab growth medium during preparation to support cell growth from the inoculum expansion stages through the production bioreactor. In addition, the cholesterol lipid concentrate was added to the production bioreactor as fixed bolus feed additions on process days 0 and 4. Operational and in-process controls for cell culture media and feeds are described in Table 18.

TABLE 18
Cell Culture Media and Feeds Preparation and Storage Controls

| Cell Culture | Operational/ | | |
|---|---|---|---|
| Media and Feeds | In-Process Controls | Target | AR |
| CDM4Mab | pH | 7.00 | 6.95-7.40 |
| (liquid growth medium) | Osmolality (mOsm/kg) | N/A | 280-320 |
| | Turbidity (NTU) | ≤4.1 | N/A |
| | Post-filtration storage | N/A | ≤30 |
| | at 2-8° C. (days) | | |
| | Post-filtration storage at | N/A | ≤36 |
| | room temperature (hrs^{)a} | | |
| | Hold time at | N/A | ≤36 |
| | operating | | |
| | temperature (hrs)b | | |
| BalanCD CHO Feed | pH | 7.00 | 7.00-7.40 |
| 4 (liquid feed | Osmolality (mOsm/kg) | 1385 | 1360-1410 |
| medium) | Post-filtration storage at | N/A | <21 |
| | room temperature (days)^{a} | | |
| | Post-filtration storage | N/A | ≤18 |
| | at 2-8° C. (days) | | |
| Glucose Feed | Post-filtration storage at | N/A | ≤69 |
| | room temperature (days)^{a} | | |
| Glutamine Feed | Osmolality (mOsm/kg) | N/A | 180-220 |
| | Post-filtration storage | N/A | ≤14 |
| | at 2-8° C. (days) | | |
| Sodium Hydroxide | Conductivity (mS/cm) | N/A | 140-190 |
| | Post-filtration storage at | N/A | <31 |
| | room temperature (days)^{a} | | |
| Cholesterol Lipid | pH | Report only (vendor | |
| Concentrate 1000X | | CoA) | |
| | Osmolality (mOsm/kg) | Report only (vendor | |
| | | CoA) | |
| | Storage at 2-8° C. (days) | N/A | ≤526 |

^{a}Room temperature at SBL: 17-25° C.
bOperating temperature: 37.0° C. (36.5-37.5° C.)

### Inoculum Expansion

Inoculum expansion steps included thaw of the WCB vial and growth in shake flasks and/or cellbags of increasing size and volume to provide sufficient cell mass to inoculate the seed bioreactor stages. The steps are performed by growth in inoculum expansion growth medium (CDM4Mab). To initiate the process, a vial of WCB G140/R603/WCB001 was thawed in pre-warmed water in a 37.0° C. water bath. The thawed vial contents were transferred into pre-warmed medium and diluted to achieve a target seed density of 0.55×10⁶ viable cells/mL.

The culture was placed in an initial 125 mL shake flask and grown in a shaking incubator at 37.0° C./5.0% CO₂ for 1 day. Every 2 to 3 days, the culture was expanded into shake flasks of larger volume and/or to multiple shake flasks. At each stage, a seeding density of 0.30×10⁶ viable cells/mL was targeted. The final inoculum preparation stage consisted of a 50 L cellbag. After 2 to 3 days of growth, the viable cell density was checked and culture was further processed to the seed bioreactor stages. Operational and in-process controls for inoculum expansion are described in Table 19.

TABLE 19
Inoculum Expansion Controls

| Process | | Tar- | | |
|---|---|---|---|---|
| Step | Process Parameter | get | NOR | AR |
| Stage 1: | Operational Parameters | | | |
| 125 mL | Vial thaw duration (min) | N/A | ≤3 | ≤3 |
| shake | Incubator temperature (° C.) | 37.0 | 36.5-37.5 | 36.5-37.5 |
| flask | Incubator CO₂ level (%) | 5.0 | 4.0-6.0 | 4.0-6.0 |
| | Agitation rate (rpm)^{a} | 96 | 91-101 | 91-101 |
| | Culture duration (hrs) | 24 | 18-30 | 18-35 |
| | Performance parameters | | | |
| | Initial viable cell density | 0.55 | 0.40-0.70 | 0.40-0.90 |
| | (×10⁶ cells/mL) | | | |
| | Final viable cell density | ≥1.80 | ≥1.60 | ≥1.60 |
| | (×10⁶ cells/mL) | | | |
| | Final cell viability (%) | ≥90.0 | ≥86.0 | ≥86.0 |
| Stage 2: | Operational Parameters | | | |
| 500 mL | Incubator temperature (° C.) | 37.0 | 36.5-37.5 | 36.5-37.5 |
| shake | Incubator CO₂ level (%) | 5.0 | 4.0-6.0 | 4.0-6.0 |
| flask | Agitation rate (rpm)^{a} | 96 | 91-101 | 91-101 |
| | Culture duration (hrs) | 72 | 48-78 | 48-91 |
| | Performance parameters | | | |
| | Initial viable cell density | 0.30 | 0.20-0.40 | 0.10-0.40 |
| | (×10⁶ cells/mL) | | | |
| | Final viable cell density | N/A | ≥3.00 | ≥2.40 |
| | (×10⁶ cells/mL) | | | |
| | Final cell viability (%) | ≥90.0 | ≥86.0 | ≥86.0 |
| Stage 3: | Operational Parameters | | | |
| 3 L | Incubator temperature (° C.) | 37.0 | 36.5-37.5 | 36.5-37.5 |
| shake | Incubator CO₂ level (%) | 5.0 | 4.0-6.0 | 4.0-6.0 |
| flask | Agitation rate (rpm)^{a} | 96 | 91-101 | 91-101 |
| | Culture duration (hrs) | 72 | 48-78 | 48-94 |
| | Performance Parameters | | | |
| | Initial viable cell density | 0.30 | 0.20-0.40 | 0.18-0.40 |
| | (×10⁶ cells/mL) | | | |
| | Final viable cell density | N/A | ≥3.00 | ≥2.98 |
| | (×10⁶ cells/mL) | | | |
| | Final cell viability (%) | ≥90.0 | ≥86.0 | ≥86.0 |
| Stage 4: | Operational Parameters | | | |
| 3 × 3 L | Incubator temperature (° C.) | 37.0 | 36.5-37.5 | 36.5-37.5 |
| shake | Incubator CO₂ level (%) | 5.0 | 4.0-6.0 | 4.0-6.0 |
| flasks | Agitation rate (rpm)^{a} | 96 | 91-101 | 91-101 |
| | Culture duration (hrs) | 72 | 48-78 | 48-78 |
| | Performance Parameters | | | |
| | Initial viable cell density | 0.30 | 0.20-0.40 | 0.16-0.40 |
| | (×10⁶ cells/mL) | | | |
| | Final viable cell density | N/A | ≥3.00 | ≥3.00 |
| | (×10⁶ cells/mL) | | | |
| | Final cell viability (%) | ≥90.0 | ≥86.0 | ≥86.0 |
| Stage 5: | Operational Parameters | | | |
| 50 L | Temperature (° C.) | 37.0 | 36.0-38.0 | 36.0-38.0 |
| cellbag | CO₂ level (%) | 5.0 | 4.0-6.0 | 4.0-6.0 |
| | Agitation rate (rpm) | 25 | 23-27 | 23-27 |
| | Culture duration (hrs) | 72 | 48-78 | 48-78 |
| | Performance Parameters | | | |
| | Initial viable cell density | 0.30 | 0.20-0.60 | 0.20-0.60 |
| | (×10⁶ cells/mL) | | | |
| | Final viable cell density | N/A | ≥3.00 | ≥3.00 |
| | (×10⁶ cells/mL) | | | |
| | Final cell viability (%) | ≥90.0 | ≥86.0 | ≥86.0 |

^{a}Shaker platform throw radius dependent parameter. Shaker platform throw radius: 22 mm

### Seed Bioreactors

The seed bioreactor stages further increase the volume and cell culture biomass prior to inoculation of the production bioreactor. The medium used in these stages was inoculum expansion growth medium (CDM4Mab). The seed bioreactor stages were 120 L, 600 L, and 3000 L stainless steel bioreactors. The bioreactors were equilibrated after media addition. Dissolved oxygen and pH probes were calibrated prior to use. Initial bioreactor set points included temperature, pH, dissolved oxygen, and agitation rate. Each bioreactor was inoculated with cell culture from the preceding stage and the culture was grown for 2 to 3 days. Seed bioreactor operational and in-process controls are summarized in Table 20.

TABLE 20
Seed Bioreactor Controls

| Process | | Tar- | | |
|---|---|---|---|---|
| Step | Process Parameter | get | NOR | AR |
| Seed | Operational Parameters | | | |
| Biore- | Temperature (° C.) | 37.0 | 36.5-37.5 | 36.5-37.5 |
| actor 1: | Agitation (rpm)^{a} | 100 | 95-105 | 95-105 |
| 120 L | pH | 7.10 | 6.75-7.45 | 6.75-7.47 |
| | Dissolved oxygen (%) | 40.0 | 20.0-100.0 | 20.0-100.0 |
| | Culture duration (hrs) | 72 | 48-78 | 48-112 |
| | Performance Parameters | | | |
| | Initial viable cell density | 0.50 | 0.40-0.70 | 0.16-0.90 |
| | (×10⁶ cells/mL) | | | |
| | Final viable cell density | N/A | ≥3.00 | ≥1.50 |
| | (×10⁶ cells/mL) | | | |
| | Final cell viability (%) | ≥90.0 | ≥89.0 | ≥89.0 |
| Seed | Operational Parameters | | | |
| Biore- | Temperature (° C.) | 37.0 | 36.5-37.5 | 36.5-37.5 |
| actor 2: | Agitation (rpm)^{a} | 74 | 70-78 | 70-78 |
| 600 L | pH | 7.10 | 6.75-7.45 | 6.75-7.50 |
| | Dissolved oxygen (%) | 40.0 | 20.0-100.0 | 20.0-100.0 |
| | Culture duration (hrs) | 72 | 48-78 | 47-87 |
| | Performance Parameters | | | |
| | Initial viable cell density | 0.50 | 0.40-0.70 | 0.17-0.90 |
| | (×10⁶ cells/mL) | | | |
| | Final viable cell density | N/A | ≥3.00 | ≥3.00 |
| | (×10⁶ cells/mL) | | | |
| | Final cell viability (%) | ≥90.0 | ≥89.0 | ≥89.0 |
| Seed | Operational Parameters | | | |
| Biore- | Temperature (° C.) | 37.0 | 36.5-37.5 | 36.5-37.5 |
| actor 3: | Agitation (rpm)^{a} | 50 | 47-53 | 47-53 |
| 3000 L | pH | 7.10 | 6.75-7.45 | 6.75-7.45 |
| | Dissolved oxygen (%) | 40.0 | 20.0-100.0 | 20.0-100.0 |
| | Culture duration (hrs) | 72 | 48-78 | 46-78 |
| | Performance Parameters | | | |
| | Initial viable cell density | 0.50 | 0.40-0.70 | 0.40-0.90 |
| | (×10⁶ cells/mL) | | | |
| | Final viable cell density | N/A | ≥3.00 | ≥3.00 |
| | (×10⁶ cells/mL) | | | |
| | Final cell viability (%) | ≥90.0 | ≥89.0 | ≥89.0 |

^{a}Agitation is dependent on volumetric scale and bioreactor impeller. Target (NOR/AR) are equivalent to power/volume 20 (17-23) W/m³ at SBL.

### Production Bioreactor

The production bioreactor stage is the final cell culture process stage which further increases the volume and mass of the cell culture for expression of the TG-1101 antibody with acceptable product quality. The basal medium used in this stage was growth medium (CDM4Mab) and feed additions were performed at specified days or criteria during the process. The production bioreactor was a 15,000 L stainless steel bioreactor.

The production bioreactor was equilibrated after media addition. Inoculum expansion medium was added to an initial volume target. Dissolved oxygen and pH probes were calibrated prior to use. Initial bioreactor set points included temperature, PH, dissolved oxygen, and agitation rate. The bioreactor was inoculated with cell culture from the N-1 seed bioreactor at a viable cell density target of 0.5×10⁶ viable cells/mL. During the production bioreactor process, pH was controlled with base addition and CO₂ sparging on demand. Dissolved oxygen was controlled with oxygen and air sparging on demand. Antifoam was added as needed to mitigate foaming concerns. Offline PH, pCO₂, pO₂, osmolality, and metabolites were monitored daily as were viable cell density (VCD) and viability. The BalanCD CHO Feed4 was added on Days 3, 5, 7, 9 at specified volumes (4.0% of initial bioreactor working volume). A calculated volume of glucose solution was fed (to 4.00 g/L) when daily bioreactor sample measured values were <3.00 g/L on specified process days. A fixed volume of glutamine solution was bolus fed on Day 3 (3.0% of initial bioreactor working volume), and a calculated volume was fed (to 4.00 mM) when daily bioreactor sample measured values were <3.00 mM on specified process days. The production bioreactor was harvested based on either culture duration or cell viability criteria, whichever occurred first.

Unprocessed bulk samples were removed prior to the clarification unit operation for adventitious agent testing as described in Example 4. Production bioreactor operational and in-process controls are described below in Table 21.

TABLE 21
Production Bioreactor Controls

| Process | | Tar- | | |
|---|---|---|---|---|
| Step | Process Parameter | get | NOR | AR |
| Produc- | Operational Parameters | | | |
| tion | Initial temperature (° C.)^{a} | 37.0 | 36.5-37.5 | 36.5-37.5 |
| Biore- | Temperature after 1st | 35.0 | 34.5-35.5 | 34.5-35.5 |
| actor: | shift (° C.)^{a} | | | |
| 15000 L | 1st temperature shift | 24 | 18-30 | 17-33 |
| | timing (hrs) | | | |
| | Temperature after 2nd | 32.5 | 32.0-33.0 | 32.0-33.0 |
| | shift (° C.)^{a} | | | |
| | 2^{nd} temperature | 72 | 66-77 | 62-77 |
| | shift timing (hrs) | | | |
| | pH at pre-inoculation | 7.40 | 7.30-7.45 | 7.30-7.45 |
| | pH at post-inoculation | 7.10 | 6.77-7.43 | 6.77-7.51 |
| | pH shift timing (hrs) | 72 | 66-77 | 62-77 |
| | pH after shift 1 | 6.70 | 6.57-6.83 | 6.54-6.96 |
| | Agitation (rpm)^{b} | 54 | 51-57 | 51-65 |
| | Dissolved oxygen (%) | 40.0 | 20.0-100.0 | 20.0-100.0 |
| | BalanCD CHO Feed 4 | 4.0 | 1.8-4.1 | 1.8-4.1 |
| | addition amount (% of | | | |
| | initial working volume)^{c} | | | |
| | Glutamine feed amount | 3.0 | N/A | 3.0 |
| | on Day 3 (% of initial | | | |
| | working volume) | | | |
| | Glutamine concentration | <3.00 | N/A | <3.00 |
| | for initiating | | | |
| | feeding (mM)^{d} | | | |
| | Glutamine concentration | 4.00 | N/A | 4.00 |
| | after feeding (mM)^{e} | | | |
| | Glucose concentration | <3.00 | N/A | <3.00 |
| | for initiating | | | |
| | feeding (g/L)^{f} | | | |
| | Glucose concentration | 4.00 | N/A | 4.00 |
| | after feeding (g/L)^{e} | | | |
| | Antifoam addition | 400 | 380-420 | 380-420 |
| | amount (g)^{g} | | | |
| | Culture duration (hrs)^{h} | 312 | 304-320 | 275-329 |
| | Performance Parameters | | | |
| | Initial viable | 0.50 | 0.40-0.70 | 0.40-0.95 |
| | cell density | | | |
| | (×10⁶ cells/mL) | | | |
| | Initial cell viability (%) | ≥95.0 | ≥89.0 | ≥89.0 |
| | Final cell viability (%)^{h} | N/A | ≥20 | ≥20 |
| | pCO₂ (mmHg) | N/A | ≤300.0 | ≤300.0 |
| | Total cell age | N/A | N/A | ≤57 |
| | (cell doublings | | | |
| | from MCB thaw) | | | |

^{a}Temporary excursions are allowed.
^{b}Agitation is dependent on volumetric scale and bioreactor impeller. Target (NOR) are equivalent to power/volume 50 (42-59) W/m³ at SBL. AR is equivalent to power/volume 42-70 W/m³.
^{c}Fed on Days 3, 5, 7, 9.
^{d}Fed on Days 4-7 as needed.
^{e}Glucose/glutamine are not measured after glucose/glutamine feed addition; values refer to target concentrations used in the calculation for determining the glucose/glutamine feed amount required.
^{f}Fed on Days 3-12 as needed.
^{g}Antifoam amount is calculated based on initial working volume of production bioreactor. Target is addition amount at one time.
^{h}Harvest occurs when either culture duration reaches its target/NOR, or when final cell viability drops lower than its NOR, whichever occurs first. As a result, it is possible for harvest to occur when final cell viability is below its NOR, or culture duration is below its NOR.

### Downstream Process and Process Controls

A process flow diagram of the downstream steps including operational controls and in-process controls are provided in Table 22. As shown in Table 22, in-process controls were incorporated into the process. Among other controls, bioburden and endotoxin were measured during multiple stages of the downstream process.

TABLE 22
Downstream Operational and In-Process Controls

| Operational Controls, Target | Process Step | In-Process Controls |
|---|---|---|
| 4900 rpm bowl speed | Harvest Clarification | Harvest bioburden |
| 5000 L/h feed flow | Centrifuge | Harvest endotoxin |
| 99 m² POD filter area | A1HC POD Depth Filter | |
| | 1.2/0.5 μm, 0.45/0.2 μm Filters | |
| | ↓ | |
| 5-34 g/L resin load | Protein A Capture Column | Column equilibration |
| 140 × 19.5 cm column | MabSelect SuRe | bioburden/endotoxin |
| | | Eluate bioburden/endotoxin |
| | ↓ | |
| 120-180 min incubation | Viral Inactivation | Pool bioburden |
| 25° C. incubation | Solvent/Detergent (SDVI) | Pool endotoxin |
| | TnBP/Polysorbate 80 | |
| | ↓ | |
| 5-65 g/L resin load | Cation Exchange Column | Column equilibration |
| 140 × 17.7 cm column | (CEX) | bioburden/endotoxin |
| | SP Sepharose FF | Load bioburden/endotoxin |
| | ↓ | |
| 200-700 g/L load | Anion Exchange Membrane | Membrane equilibration |
| | Chromatography (AEX) | bioburden/endotoxin |
| | Mustang Q | Load bioburden/endotoxin |
| | ↓ | |
| ≤400 g/m² load | Viral Filtration (VF) | VF filter equilibration |
| | Viresolve ® | bioburden/endotoxin |
| | | Pre, post-use filter integrity |
| | | Load bioburden, endotoxin |
| | ↓ | |
| ≤200 g/m² membrane load | Ultrafiltration/Diafiltration | Membrane equilibration |
| 39 mg/mL concentration | (UFDF) | bioburden/endotoxin |
| | Pellicon 2 Biomax | Load bioburden/endotoxin |
| | ↓ | |
| Polysorbate 80 addition to | Formulation | Formulated bulk pool |
| 0.07% and ublituximab | (Polysorbate 80 addition), | bioburden |
| concentration at 25 mg/mL | 0.2 μm filtration | Filtered formulated DS |
| in final formulation | | bioburden^{a} |
| | | Post-use filter integrity |
| | ↓ | |
| 5.00-6.00 kg per bag | Fill, Freeze and Store | Freezing duration, storage |
| | ≤−35° C. | temperature, storage |
| | | duration |

^{a}Beginning, middle and end of filling operation

### Harvest Clarification

The cell culture supernatant was harvested and clarified from the 15,000 L bioreactor to remove cells and cell debris. The clarification was performed using a continuous centrifugation followed by depth filtration.

The harvest clarification step was operated in a room in which the controlled temperature range was 17-25° C.; the harvest pool vessel was a jacketed tank, which maintains the pool at 2-8° C. The centrifuge shot interval was set based on the packed cell volume percentage (PCV) and adjusted to allow for 80% bowl fill. The flow rate to the centrifuge was actively controlled; the filtration feed flow was the same as the centrifuge feed flow. Process parameters included centrate back pressure, the depth filtration operating pressure, inlet pressure, harvest weight, bioburden and endotoxin.

The centrate was clarified by use of a three-stage filtration process as required to pass through a 0.2 μm filter (Millistak A1HC POD depth filters, 1.2/0.5 μm filters and 0.45/0.22 μm sterilizing grade filters). Before use, the filters were flushed with WFI followed by equilibration. The centrate was pumped through the filters, which are monitored to ensure acceptable back pressure. Air was used to expel the contents in the filters followed by a buffer flush. The clarified harvest was stored at 2 to 8° C. for ≤11 days.

### Protein A Column Chromatography (ProA)

Protein A column chromatography was performed using MabSuRe Select resin (Cytiva) in bind/elute mode. This step provided capture and purification of TG-1101 with reduction of process impurities such as cell culture components, HCP, and residual DNA, as well as for the provision of viral safety.

The packed column was assessed for HETP performance using sodium acetate/benzyl alcohol buffer. During manufacturing, all column operations were performed at 13 to 25° C. Before loading, the column was sanitized with 0.5 M sodium hydroxide and flushed with WFI. The column was equilibrated with Equilibration Buffer (25 mM Tris, 25 mM NaCl, 5 mM EDTA, pH 7.1). The clarified harvest was mixed briefly and then loaded onto the column using a maximum of 36 g TG-1101/L resin load and the column was washed with Wash 1 Buffer (equilibration buffer) followed by a second wash with a high salt, Wash 2 Buffer (25 mM Tris, 1.2 M NaCl, 5 mM EDTA, pH 7.1), followed by an additional wash using Wash 3 Buffer (equilibration buffer). Bound TG-1101 was eluted 200-220 cm/h with elution buffer (25 mM sodium citrate, pH 3.6) using elution peak collection by A280, not to exceed 2.4 column volumes. The eluate was collected in a tank containing neutralization buffer (2.0M Tris, pH 7.5) and filtered via a 0.2 μm filter before transfer to a different tank. The Protein A column was sanitized with 0.5M sodium hydroxide. Up to three cycles per batch may be run; if multiple cycles were required for the Protein A process, the column was re-equilibrated for the next cycle with equilibration buffer. After sanitization, the Protein A column was neutralized with equilibration buffer and stored at 13 to 25° C. in 200 mM sodium acetate, 2% benzyl alcohol, pH 5.0. The pooled (if more than one cycle), neutralized eluate was diluted with 5 mM Sodium Phosphate, pH 7.2 to a concentration of ≤10 g/L and stored at 13-25° C. for ≤24 hours or at 2 to 8° C. for ≤11 days.

### Solvent Detergent Viral Inactivation (SDVI)

The Protein A capture chromatography step was followed by a solvent detergent viral inactivation (SDVI) step to inactivate potential viral agents. In the currently validated manufacturing process, the Protein A elution pool was diluted and treated with 3.5% (v/v) TnBP, 12% (w/v) polysorbate 80 and held at 24.0-26.0° C. for at least 120 minutes while mixing. The SDVI pool was filtered with a 0.2 μm filter before transfer to a different tank, where it was diluted with 5 mM sodium phosphate, pH 7.2 to 50 mOsm/kg, and the pH adjusted to 7.2 as needed. After pH adjustment, the pool was held at 13 to 25° C. for ≤30 hours before proceeding to the CEX column.

### Cation Exchange Chromatography (CEX)

Cation exchange column chromatography was performed using SP Sepharose Fast Flow (Cytiva) in bind/elute mode. This step provided further purification of TG-1101, removing residual process impurities (HCPs, DNA, residual polysorbate 80, and TnBP).

The packed column was assessed for HETP performance using a sodium acetate/benzyl alcohol-containing buffer. During manufacturing, all column operations were performed at 13 to 25° C. In the currently validated manufacturing process, before loading, the column was sanitized with 0.5 M sodium hydroxide and rinsed with WFI. The column was equilibrated using equilibration buffer (20 mM sodium phosphate, pH 7.2). The viral inactivated/diluted solution was loaded onto the column at a maximum of 65 g/L resin load. The column was washed with Wash 1 Buffer (equilibration buffer) followed by a second wash with Wash 2 Buffer (equilibration buffer in the reverse direction). Bound TG-1101 was eluted using 20 mM sodium phosphate, 150 mM NaCl, pH 7.2 and elution peak collection by A280 monitoring. The eluate was filtered (0.2 μm) and stored at 13 to 25° C. for ≤72 hours or at 2 to 8° C. for ≤11 days. After elution, the column was stripped with 2 M NaCl followed by sanitization with 0.5 M sodium hydroxide. One cycle per batch was allowed. After completion, the column was sanitized (0.5 M sodium hydroxide) and stored in storage buffer (200 mM sodium acetate, 2% benzyl alcohol, pH 5.0).

### Anion Exchange Membrane Chromatography (AEX)

Anion exchange membrane chromatography was performed using a Mustang Q (Pall Corporation) membrane absorber (MA) filter in flow-through mode. This step provided further purification of the TG-1101; the product flowed through the membrane and remaining impurities (DNA, HCP and viruses) were retained on the membrane. The membrane was single use (i.e., each individual membrane cannot be reused) and several membrane capsules may be used per batch at the appropriate loading level.

To perform the step, in the currently validated manufacturing process, eluate from the cation exchange column chromatography step was diluted with 20 mM sodium phosphate, pH 8.0, followed by the adjustment of the pH to 8.0. The concentration was determined and the number of cycles calculated based on protein concentration such that the loading was 200 to 700 g TG-1101/L membrane load. The membrane was sanitized with 0.5 M sodium hydroxide, flushed with 2 M NaCl then with WFI, before being equilibrated with Equilibration Buffer (20 mM sodium phosphate, 75 mM NaCl, pH 8.0) in preparation for the load. After loading, the membrane was chased with 20 mM sodium phosphate, 75 mM NaCl, pH 8.0, to maximize recovery. The collected flow-through containing product from all cycles was filtered (0.5/0.2 μm), diluted to ≤6 g/L with 75 mM sodium citrate, 312 mM NaCl, pH 6.0, and the pH is adjusted to 6.8. The adjusted AEX pool was stored at 13 to 25° C. for ≤72 hours or at 2 to 8° C. for ≤11 days.

### Viral Filtration (VF)

Viral filtration was performed by filtering through a Viresolve prefilter in series with a Viresolve Pro viral filter (Millipore Sigma), at an operating temperature Target Range of 13-25° C. The process used sufficient filters to meet the loading limit. The step was designed to remove potential viruses, including small viruses such as parvovirus.

To perform the filtration, the filters were set up in series and flushed with WFI, integrity tested, then sanitized with 0.5 M sodium hydroxide. This was followed by flushing with equilibration buffer (25 mM sodium citrate, 154 mM NaCl, pH 6.5). The filtered Mustang Q membrane flow-through was processed through the viral reduction filters.

Protein concentration was determined and used to confirm the membrane load ratio was ≤600 g/m². After loading, the membrane was chased with equilibration buffer and post-use integrity testing was performed. Viral filtrate was stored at 13 to 25° C. for ≤72 hours or at 2 to 8° C. for ≤11 days.

### Ultrafiltration/Diafiltration (UFDF) Controls

The UFDF step was used to concentrate the viral filtrate and buffer exchange into diafiltration buffer at an operating temperature Target Range of 13-25° C. The process used a tangential flow filter with a 30 kDa molecular weight cut-off. The setup included up to sufficient filters to meet the loading limit of ≤250 g/m². To perform the operation, the membranes were sanitized with 0.5M NaOH, flushed with WFI, and flushed/equilibrated with diafiltration buffer before the start of the unit operation.

The unit operation included an initial ultrafiltration (UF1) step in which the TG-1101 was first concentrated to a target of 39 mg/mL. This was followed by diafiltration (DF) into diafiltration buffer (25 mM sodium citrate 154 mM NaCl, pH 6.5) with 8 diavolumes with an upper limit of ≤9.2 diavolumes. The ultrafiltration system was flushed with diafiltration buffer to maximize recovery; the flush was transferred to the formulation vessel and combined with the UF/DF Pool. The membranes were flushed with WFI, cleaned with 0.5M NaOH, 250 ppm sodium hypochlorite, flushed with WFI, and stored in 0.1 M sodium hydroxide. The diluted UFDF pool was stored at 15 to 25° C. for ≤18 hours.

Formulation. Filtration, and Filling

The formulation step included the addition of concentrated polysorbate 80 in formulation buffer to achieve the final drug substance formulation, at an operating temperature Target Range of 17-25° C. The step was performed by adding the stabilization buffer (25 mM sodium citrate 154 mM NaCl, 10 g/L polysorbate 80, pH 6.5). After the addition of the stabilization buffer, the pool was diluted to a target of 23.5 to 26.5 mg/mL with 25 mM sodium citrate 154 mM NaCl, 700 mg/L polysorbate 80, pH 6.5, resulting in a ready-to-fill drug substance in the formulation buffer of 25 mM sodium citrate, 154 mM NaCl, 0.07% polysorbate 80, pH 6.5.

The formulated bulk drug substance was transferred and 0.2 μm filtered into 6 L Celsius® FFT bags in a closed, single use system to a target fill volume of 5.50 L. After filling, the formulated bulk drug substance was frozen at ≤−60 for >17 hours and stored at ≤−35° C.

### Example 4: Detection of Viruses or Adventitious Agents in 15,000 Commercial-Scale Production of TG-1101 in YB2/0 Rat Hybridoma Cells

Adventitious agent and viral safety of TG-1101 were assured through several strategies in a multi-pronged approach. The strategies included preventing the introduction of adventitious agents into the manufacturing process, clearing them with dedicated processing steps, and testing for them at appropriate points in the process. Additionally, testing for adventitious virus was performed to confirm the absence of adventitious virus during cell line/cell bank development and manufacturing. Viral clearance studies confirmed the capacity of the manufacturing process to clear virus.

During the manufacture of TG-1101, a comprehensive viral safety strategy consisted of the following approaches and activities:

### Raw Material Sourcing

There were no raw materials of animal or biological origin directly used in the TG-1101 upstream manufacturing process. For example, cell culture media and feed used during manufacturing, CDM4Mab cell growth media and BalanCD-CHO Feed 4, are proprietary, commercially available cell culture media. Both media were animal-component free, and no components are identified as being in contact with a raw material of animal origin in its manufacture. During the downstream manufacturing process, the protein A chromatography resin and was not derived from material of animal original and cationic chromatography resin did not contain any raw materials from animal origin.

### Testing of the Production Cell Line During Development and the Subsequently Derived Cell Banks (MCB, WCB, And EPCB)

The cell bank system for TG-1101 was a two-tiered system made of one master cell bank (MCB) and working cell banks (WCB). Cell bank (i.e., MCB and WCB) adventitious agent testing included mycoplasmal, microbial, and non-endogenous virus and adventitious virus testing. Additional adventitious agent testing (microbial and viral) was performed on the end of production cell bank (EPCB) derived from the WCB.

### Adventitious Agent Evaluation During TG-1101 Drug Substance Manufacturing

Specifically, routine mycoplasmal, microbial, and viral adventitious testing of the unprocessed bulk harvest (from the production bioreactor) was conducted, as well as in-process bioburden and endotoxin testing thoughout the manufacturing process.

### Viral Clearance Studies

Testing for viral clearance in representative scale down models demonstrated the capacity of the manufacturing process to inactivate and/or eliminate model viruses. Viral clearance studies were performed using four model viruses, including Xenotropic Murine Leukemia Virus (XMuLV), a specific model virus for the retroviral family. Three additional non-specific model viruses were chosen to provide a range of virus properties and sizes as well as resistance to chemical inactivation. Minute Virus of Mice (MVM), a model for parvoviruses, was chosen as a virus representing small size and high resistance to chemical inactivation and is also a potential virus for contamination of mammalian cell lines. Psudorabies virus (PRV) and Reovirus Type 3 (Reo 3) were also tested for viral clearance.

The TG-1101 manufacturing process included specific, orthogonal, dedicated viral inactivation/removal steps including a solvent/detergent inactivation step and a viral filtration step. The manufacturing process included several chromatography steps, which also contributed to virus inactivation/removal during the manufacturing process. Collectively, the adventitious agent controls in the manufacturing process provided a high degree of assurance that the TG-1101 drug substance has adequate safety in terms of adventitious agents.

### Example 5: Impact of Post-Shift pH on Process Performance and Product Quality of TG-1101 Upstream Manufacturing Process

### Background

Throughout the course of development of the upstream portion of the commercial TG-1101 manufacturing process (“Process C”), numerous experiments and data analysis were performed. Culture pH was identified as an important parameter capable of impacting process performance and product quality. pH control of the commercial manufacturing process run (15,000 L) at Samsung Biologics (SBL) (“Process C2”, commercial process) follows a control strategy as shown in Table 23.

TABLE 23
pH Control Strategy for Process C2

| Process Step | Process Parameter | Target | AR^{a} |
|---|---|---|---|
| Production | pH at pre-inoculation | 7.40 | 7.30-7.45 |
| Bioreactor | pH at post-inoculation | 7.10 | 6.77-7.51 |
| | (pH1) | | |
| | pH shift timing (hrs) | 72 | 62-77 |
| | pH after shift (pH2) | 6.70 | 6.54-6.96 |

^{a}AR = acceptable range

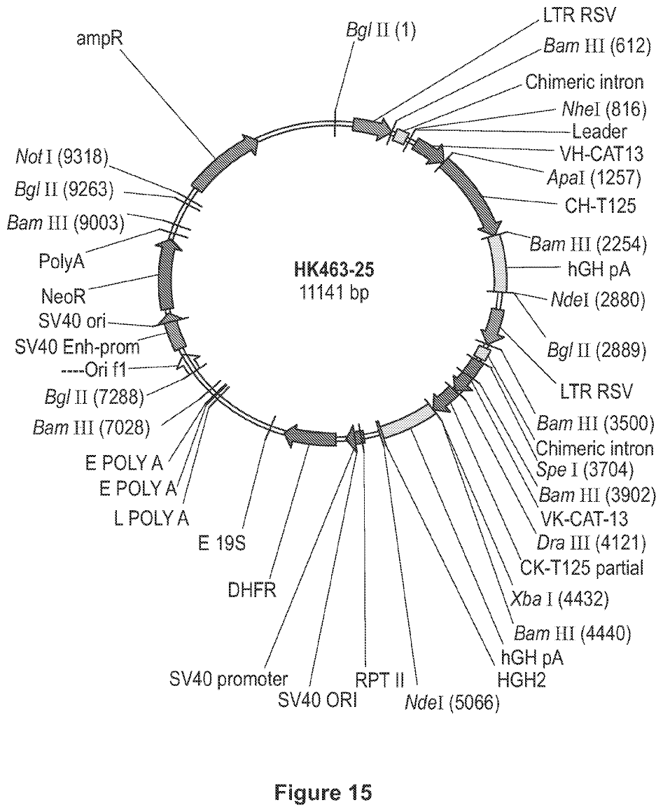

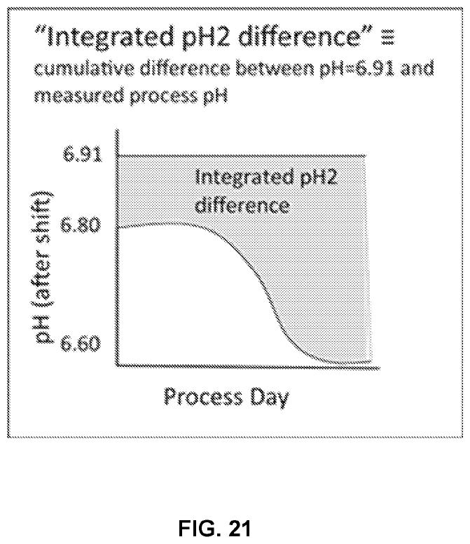

Through both 15 kL and 20 kL GMP manufacturing results, as well as scale-down model (10 L) results, the cumulative cell exposure to pH after the day 3 (72 hours) pH shift was identified as particularly important to process performance. This latter calculated parameter is defined as “integrated pH2 difference” and can be conceptually seen as the shaded section shown in FIG. 21.

### Materials and Methods

### Cell Culture

The mammalian cell expression system used was the TG-1101 YB2/0 rat hybridoma cell line expanded from the working cell bank lot #G140/R603/WCB001 or lot #127646, described above. Upon cryovial thaw of cells from either WCB, separate cell expansions were performed in shaker flask vessels and disposable cellbag bioreactors of increasing volume. Cells were expanded in CDM4Mab (Cytiva) cell culture medium supplemented to 1× cholesterol levels using 1000× cholesterol (Thermo) for multiple passages. After further cell expansion in seed bioreactor cultures, when enough cell inoculum was generated, production cultures were operated in fed-batch mode. During fed-batch production culture, BalanCD CHO Feed 4 (Fuji) was added at fixed volumes on process days 3, 5, 7, and 9. In addition, an obligatory glutamine feed addition was added on Day 3. Glutamine and glucose feeds were added according to pre-established criteria. In general, the cell culture process conditions were similar across each of the different cultures analyzed, with the exception of normal, or purposely-introduced drift in culture pH. Cell culture growth medium, feed medium, and feed solutions were prepared in a manner consistent with the spirit of GMP manufacturing.

In-process monitoring was performed throughout the duration of the production bioreactor cultures. Included amongst these samples were daily measurements for viable cell density (VCD) and cell viability by an automated cell counter (ViCell XR, Beckman Coulter). Nova Flex2 (Nova Biomedical) instrument was used for metabolite measurements (glucose, lactate, glutamine, ammonia). Nova pHOx (Nova Biomedical) and/or Siemens blood gas analyzers were used for measuring offline pH, pO₂, and pCO₂ as needed. Nova Flex 2 (Nova Biomedical), and an Advanced Instruments osmometer were used for measuring osmolality. For titer and product quality measurements, cells were removed from the culture, clarified through centrifugation, and kept in storage until ready to be analyzed.

### Protein Analytical and Functional Assays

### N-Glycans

N-linked glycans are cleaved from the product by enzymatic deglycosylation using PNGase F and labeled with 2-aminobenzamide (2-AB), a fluorescent compound. The labeled glycans are resolved using a hydrophilic interaction partition mode ultra-performance liquid chromatography (HILIC-UPLC) column equipped with a fluorescence detector (fluorescence excitation at 360 nm and emission at 428 nm). Blank, glycans from reference standard, and glycan standards (human IgG N-linked glycan library) are also injected to assess system suitability. Peak identification from the resulting test sample chromatograms are identified based on retention time and relative to peaks in the glycan standard which have been confirmed by mass spectrometry.

### Results and Conclusions

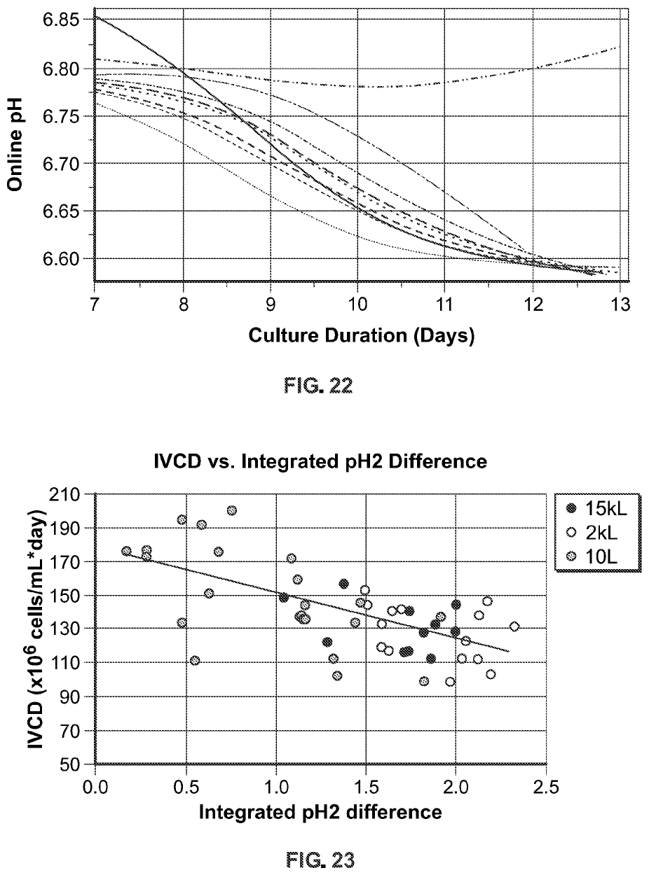

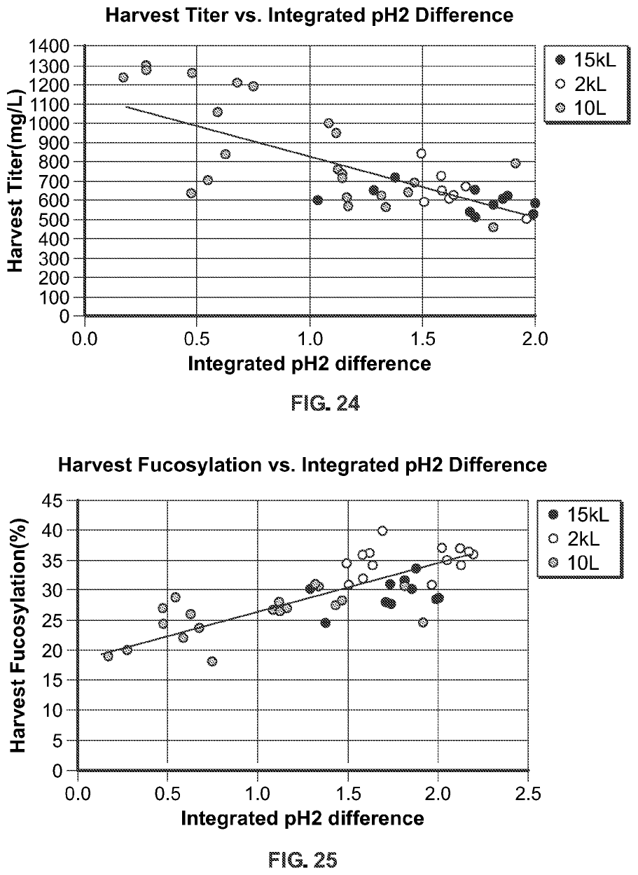

Post-shift pH (pH2) follows a trajectory that is generally consistent in terms of its trend. That is, on approximately Day 7-8 of the production bioreactor culture, lactate begins to increase to an appreciable amount whereby the culture pH drops towards the lower end of its control range. The speed and timing of this drop in pH varies between the Process C GMP manufacturing batches and 10 L scale-down model cultures (FIG. 22). As a result, the cumulative cell exposure towards pH across each of these cultures is somewhat different, and as a result, the integrated pH2 difference is also different. Integrated pH2 difference has an inverse relationship to the cumulative number of cells generated during the production bioreactor, in that the lower the integrated pH2 difference, the higher the integrated viable cell density (IVCD) (FIG. 23). As a result of the higher number of cells, integrated pH2 difference also has an inverse relationship to the titer results at harvest (FIG. 24). As a result of the higher amount of TG-1101 being constituitively secreted from the cells, the % fucosylation is generally lower in those cultures that demonstrate a lower integrated pH2 difference (FIG. 25). These results point out that if the objective is to reduce % fucosylation, such as what was done to transition the TG-1101 manufacturing process from Process A/B to Process C, this can be achieved by introducing process changes that increase TG-1101 productivity. Minimizing integrated pH2 difference is one means with which to do so.

Post-shift pH is directly controlled in the commercial TG-1101 manufacturing process using a pH control strategy that impedes % fucosylation from falling outside of the drug substance (DS) release specification. When post-shift pH is controlled within its control range at the commercial manufacturing scale, the % fucosylation results observed are relatively intransient in the 22-37% range.

### Example 6: Unique Glycosylation Signature of Anti-CD20 Antibodies Produced in YB2/0 Rat Hybridoma Cells at 15,000 L Commercial-Scale

Using the manufacturing methods disclosed herein (e.g., such as described in Example 3), anti-CD20 antibodies were produced at a 15,000 L scale from source antibody TG-1101. The anti-CD20 antibodies were found to contain a unique glycosylation profile. Without being bound by theory, the relative distribution of the various N-glycans, or individual sugar residues present in those N-glycans, can determine the biological and clinical properties of the anti-CD20 antibody composition provided herein. See, co-pending (and commonly owned) U.S. Provisional Appl. No. 63/347,852, entitled “ANTI-CD20 ANTIBODY COMPOSITIONS,” filed Jun. 1, 2022, U.S. Provisional Application Ser. No. 63/421,078, filed on Oct. 31, 2022, and U.S. Provisional Application Ser. No. 63/445,082, filed on Feb. 13, 2023, the entire contents of which are incorporated by reference herein. The anti-CD20 antibody compositions described, as well as those described herein, may be used to treat cancers (e.g., hematological cancers) and autoimmune disorders (e.g., RMS).

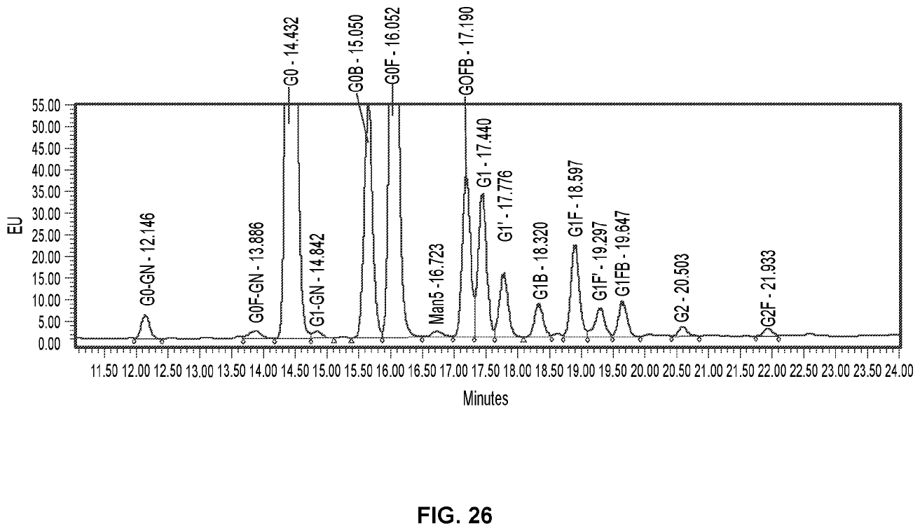

The glycosylation profile of a sample of anti-CD20 antibodies was determined by measuring fluorescently labeled N-glycans (fluorescent label is 2-aminobenzamide) that were enzymatically cleaved from the anti-CD20 antibody proteins using PNGase F. The labeled glycans were resolved using a hydrophilic interaction column equipped. the glycans flowed through a fluorescence detector after separation. Peak identification from the test sample chromatograms was identified based on retention time and relative to peaks in the glycan standard which have been confirmed by mass spectrometry. The relative percentage of each N-glycan was calculated based on the N-glycan peak area divided by the total peak area of all N-glycans. The glycosylation profile is shown in FIG. 26.

The glycosylation profile of anti-CD20 antibodies was assessed by intact mass analysis (LC-MS) under non-reducing conditions. The sample of anti-CD20 antibodies was first exchanged into MS appropriate buffers during the chromatography step using SEC and a mobile phase containing TFA, acetonitrile, and water. The sample was then introduced into an ESI-QTOF for intact mass analysis. The mass spectra are deconvoluted and the peaks are assigned based on mass. The relative abundance of each anti-CD20 antibody provided herein containing N-glycan was calculated by taking the abundance of an N-glycan and dividing by the total abundance of all identified peaks. Results are provided below in Table 24.

TABLE 24
Intact Molecular Weights for Sample of Anti-CD20 Antibodies by LC-MS

| | | Batch | | |
|---|---|---|---|---|
| | | GMP1/RM-105238 | GMP16/RS-117808 | CR |
| Mass Species | Process | B2 | C1 | C2 |
| G0/G0 | Observed MW | 146779.6 | 146781.3 | 146780.1 |
| (Theoretical MW, | Relative Abundance (%) | 14.1 | 16.9 | 18.9 |
| 146779.3) | | | | |
| G0/GF | Observed MW | 146927.5 | 146931.3 | 146929.8 |
| (Theoretical MW, | Relative Abundance (%) | 16.0 | 14.9 | 16.7 |
| 146925.4) | | | | |
| G0F/G0F | Observed MW | 147071.9 | 147073.5 | 147073.6 |
| (Theoretical MW, | Relative Abundance (%) | 18.3 | 12.3 | 13.8 |
| 147071.6) | | | | |
| G0F/G1F | Observed MW | 147235.7 | 147229.7 | 147233.7 |
| (Theoretical MW, | Relative Abundance (%) | 10.8 | 7.4 | 6.9 |
| 147233.7) | | | | |
| 1-G0/G1-GlcNAc | Observed MW | 146730.1 | 146736.4 | 146739.4 |
| (Theoretical MW, | Relative Abundance (%) | 2.1 | 2.7 | 3.1 |
| 146738.2) | | | | |
| 2-G0/G0B | Observed MW | 146980.6 | 146982.2 | 146981.3 |
| (Theoretical MW, | Relative Abundance (%) | 5.6 | 10.8 | 11.7 |
| 146982.3) | | | | |
| 3-G0F/G0B | Observed MW | ND | 147130.3 | 147128.3 |
| (Theoretical MW, | Relative Abundance (%) | | 10.5 | 9.7 |
| 147128.5) | | | | |
| 4-G0F/G0F + K | Observed MW | 147199.3 | ND | ND |
| (Theoretical MW, | Relative Abundance (%) | 7.1 | | |
| 147199.7) | | | | |
| 5-G0F/G0FB | Observed MW | ND | 147271.7 | 147273.6 |
| (Theoretical MW, | Relative Abundance (%) | | 8.3 | 7.1 |
| 147274.6) | | | | |
| 6-G0F/G0F + 2K | Observed MW | 147330.9 | ND | ND |
| (Theoretical MW, | Relative Abundance (%) | 4.0 | | |
| 147327.9) | | | | |
| 7-G0F/G1F + 1K | Observed MW | 147360.2 | ND | ND |
| (Theoretical MW, | Relative Abundance (%) | 5.1 | | |
| 147361.9) | | | | |
| 8-G1F/G1F + 1K | Observed MW | 147396.4 | 147396.3 | 147396.5 |
| (Theoretical MW, | Relative Abundance (%) | 5.8 | 4.0 | 4.2 |
| 147395.8) | | | | |
| 9-G0F/G1FB | Observed MW | 147432.0 | 147436.6 | 147436.2 |
| (Theoretical MW, | Relative Abundance (%) | 2.6 | 4.4 | 4.3 |
| 147436.7) | | | | |
| 10-G0F/G1F + 2K | Observed MW | 147488.0 | 147488.2 | ND |
| (Theoretical MW, | Relative Abundance (%) | 2.2 | 3.8 | |
| 147490.1) | | | | |
| 11-G1F/G1F + K | Observed MW | 147528.6 | 147524.9 | 147529.2 |
| (Theoretical MW, | Relative Abundance (%) | 2.4 | 2.2 | 1.9 |
| 147524.0) | | | | |
| 12-G1F/G1FB | Observed MW | 147596.2 | 147597.6 | 147600.3 |
| (Theoretical MW, | Relative Abundance (%) | 1.2 | 1.7 | 1.7 |
| 147598.7) | | | | |
| 13-G1F/G1F + 2K | Observed MW | 147653.3 | ND | ND |
| (Theoretical MW, | Relative Abundance (%) | 1.1 | | |
| 147652.2) | | | | |

Abbreviation: ND = not detected.

The anti-CD20 antibodies provided herein can be described by any one, two, three, four, five, six, seven, eight, nine, ten, eleven, twelve, thirteen, fourteen, fifteen, sixteen, seventeen, eighteen, nineteen, twenty or all of the N-glycans or individual sugar residues described in the following subsections.

In some aspects, the anti-CD20 antibodies provided herein comprise at least two, three, four, five, six, seven, eight, nine, ten, 11, 12, 13, 14, or 15 N-glycans within the following relative abundance range:
(a) 0.3% to 2% G0-GN;
(b) 0.1% to 2% G0F-GN;
(c) 0.1% to 1% G1-GN;
(d) 5% to 20% G0B;
(e) 5% to 30% G0F;
(f) 0.1% to 1.5% Man5;
(g) 1% to 15% G0FB;
(h) 1% to 13% G1;
(i) 0.5% to 10% G1′;
(j) 0.5% to 6% G1B;
(k) 0.5% to 12% G1F;
(l) 0.1% to 3% G1F;
(m) 0.1% to 3% G1FB;
(n) 0.1% to 2% G2; and
(o) 0.1% to 2% G2F.

In a more specific aspect, the anti-CD20 antibodies provided herein comprise at least two, three, four, five, six, seven, eight, nine, ten, 11, 12, 13, 14, or 15 N-glycans within the following relative abundance range:
(a) 0.8% to 1.1% G0-GN;
(b) 0.5% to 1.1% G0F-GN;
(c) 0.3% to 0.6% G1-GN;
(d) 9.5% to 14.1% G0B;
(e) 12.8% to 19.7% G0F;
(f) 0.4% to 0.7% Man5;
(g) 5.1% to 7.0% G0FB;
(h) 5.7% to 6.4% G1;
(i) 2.7% to 3.3% G1′;
(j) 1.4% to 2.0% G1B;
(k) 2.6% to 4.2% G1F;
(l) 1.1% to 1.6% G1F;
(m) 1.1% to 1.8% G1FB;
(n) 0.5% to 0.7% G2; and
(o) 0.3% to 0.5% G2F.

In an even more specific aspect, the anti-CD20 antibodies provided herein comprise at least two, three, four, five, six, seven, eight, nine, ten, 11, 12, 13, 14, or 15 N-glycans within the following relative abundance range:
(a) 0.9% G0-GN;
(b) 0.8% G0F-GN;
(c) 0.5% G1-GN;
(d) 10.9% G0B;
(e) 17.0% G0F;
(f) 0.6% Man5;
(g) 6.0% G0FB;
(h) 6.1% G1;
(i) 2.9% G1′;
(j) 1.6% G1B;
(k) 3.2% G1F;
(l) 1.3% G1F;
(m) 1.3 G1FB;
(n) 0.5% G2; and
(o) 0.3% G2F.

In some aspects, the anti-CD20 antibodies provided herein comprise an N-glycan profile comprising a relative abundance of from about 0.3% to about 2% G0-GN, from about 0.8% to about 1.1% G0-GN, or about 0.9% G0-GN. In certain embodiments, the anti-CD20 antibodies comprise an N-glycan profile comprising a relative abundance of from about 0.1% to about 2% G0F-GN, from about 0.5% to about 1.1% G0F-GN, or about 0.8% G0F-GN. In certain embodiments, the anti-CD20 antibodies comprise an N-glycan profile comprising a relative abundance of from about 0.1% to about 1% G1-GN, from about 0.3% to about 0.6% G1-GN, or about 0.5% G1-GN. In certain embodiments, the anti-CD20 antibodies comprise an N-glycan profile comprising a relative abundance of from about 5% to about 20% G0B, from about 5% to about 15% G0B, from about 9.5% to about 14.1% G0B, about 10.9% G0B, or about 10% G0B. In certain embodiments, the anti-CD20 antibodies comprise an N-glycan profile comprising a relative abundance of from about 5% to about 30% G0F, from about 12.8% to about 19.7% G0F, or about 17.0% G0F. In certain embodiments, the anti-CD20 antibodies comprise an N-glycan profile comprising a relative abundance of from about 0.1% to about 1.5% Man5, from about 0.4% to about 0.7% Man5, or about 0.6% Man5. In some embodiments, Man5 is the only high mannose N-glycan in the N-glycan profile. In certain embodiments, the anti-CD20 antibodies comprise an N-glycan profile comprising a relative abundance of from about 1% to about 15% G0FB, from about 5.1% to about 7.0% G0FB, or about 6.0% G0FB. In certain embodiments, the anti-CD20 antibodies comprise an N-glycan profile comprising a relative abundance of from about 1% to about 13% G1, from about 5.7% to about 6.4% G1, or about 6.1% G1. In certain embodiments, the anti-CD20 antibodies proteins comprise an N-glycan profile comprising a relative abundance of from about 0.5% to about 10% G1′, from about 2.7% to about 3.3% G1′, or about 2.9% G1′. In certain embodiments, the anti-CD20 antibodies comprise an N-glycan profile comprising a relative abundance of from about 0.5% to about 6% G1B, from about 1.4% to about 2.0% G1B, or about 1.6% G1B. In certain embodiments, the anti-CD20 antibodies comprise an N-glycan profile comprising a relative abundance of from about 0.5% to about 12% G1F, from about 2.6% to about 4.2% G1F, or about 3.2% G1F. In certain embodiments, the anti-CD20 antibodies comprise an N-glycan profile comprising a relative abundance of from about 0.1% to about 3% G1F, from about 1.1% to about 1.6% G1F, or about 1.3% G1F. In certain embodiments, the anti-CD20 antibodies comprise an N-glycan profile comprising a relative abundance of from about 0.1% to about 3% G1FB, from about 1.1% to about 1.8% G1FB, or about 1.3 G1FB. In certain embodiments, the population of anti-CD20 antibodies comprise an N-glycan profile comprising a relative abundance of from about 0.1% to about 2% G2, from about 0.5% to about 0.7% G2, or about 0.5% G2. In certain embodiments, the anti-CD20 antibodies comprise an N-glycan profile comprising a relative abundance of from about 0.1% to about 2% G2F, from about 0.3% to about 0.5% G2F, or about 0.3% G2F.

In some aspects, the anti-CD20 antibodies provided herein comprise an N-glycan profile comprising a relative abundance of from about 0.3% to about 2% G0-GN, from about 0.1% to about 2% G0F-GN, from about 0.1% to about 1% G1-GN, from about 5% to about 20% G0B, from about 5% to about 30% G0F, from about 0.1% to about 1.5% Man5, from about 1% to about 15% G0FB, from about 1% to about 13% G1, from about 0.5% to about 10% G1′, from about 0.5% to about 6% G1B, from about 0.5% to about 12% G1F, from about 0.1% to about 3% G1F, from about 0.1% to about 3% G1FB, from about 0.1% to about 2% G2, and from about 0.1% to about 2% G2F. In some embodiments, Man5 is the only high mannose N-glycan in the N-glycan profile.

In some aspects, the anti-CD20 antibodies provided herein comprise an N-glycan profile comprising a relative abundance of from about 0.8% to about 1.1% G0-GN, from about 0.5% to about 1.1% G0F-GN, from about 0.3% to about 0.6% G1-GN, from about 9.5% to about 14.1% G0B, from about 12.8% to about 19.7% G0F, from about 0.4% to about 0.7% Man5, from about 5.1% to about 7.0% G0FB, from about 5.7% to about 6.4% G1, from about 2.7% to about 3.3% G1′, from about 1.4% to about 2.0% G1B, from about 2.6% to about 4.2% G1F, from about 1.1% to about 1.6% G1F, from about 1.1% to about 1.8% G1FB, from about 0.5% to about 0.7% G2, and from about 0.3% to about 0.5% G2F. In some embodiments, Man5 is the only high mannose N-glycan in the N-glycan profile.

In some aspects, the anti-CD20 antibodies provided herein comprise an N-glycan profile comprising a relative abundance of about 0.9% G0-GN, about 0.8% G0F-GN, about 0.5% G1-GN, about 10.9% G0B, about 17.0% G0F, about 0.6% Man5, about 6.0% G0FB, about 6.1% G1, about 2.9% G1′, about 1.6% G1B, about 3.2% G1F, about 1.3% G1F′, about 1.3 G1FB, about 0.5% G2, and about 0.3% G2F. In some embodiments, Man5 is the only high mannose N-glycan in the N-glycan profile.

In some aspects, the anti-CD20 antibodies provided herein comprise an N-glycan profile comprising a relative abundance ratio of about 0.1 to about 0.15 G1 to G0 N-glycans. In some aspects, the anti-CD20 antibodies provided herein comprise an N-glycan profile comprising a relative abundance ratio of about 0.5 to about 0.9 G1F to G1 N-glycans.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

The contents of all cited references (including literature references, patents, patent applications, and websites) that can be cited throughout this application are hereby expressly incorporated by reference in their entirety for any purpose, as are the references cited therein.

What is claimed is:

1. A method of producing at least 10,000 L of an antibody protein in rat hybridoma cells, comprising culturing the rat hybridoma cells in a cell culture having a culture pH of about 6.5 to about 7.55, wherein the rat hybridoma cells comprise an expression vector comprising a polynucleotide encoding the antibody protein.

2. The method of claim 1, wherein the culture pH is about 6.5 to about 7.0.

3. The method of claim 2, wherein the culture pH of about 6.5 to about 7.0 is set on culture day 2 of the cell culture.

4. The method of claim 2, wherein the culture pH of about 6.5 to about 7.0 is set on culture day 3 of the cell culture.

5. The method of claim 1, wherein the culture pH is about 7.0 to about 7.55.

6. The method of claim 5, wherein the culture pH of about 7.0 to about 7.55 is set on culture days 0 to 3 of the cell culture.

7. The method of claim 5 or 6, wherein the culture pH is lowered on day 2 or day 3 of the cell culture to about 6.5 to about 7.0.

8. The method of claim 7, wherein the culture pH is lowered on day 3 of the cell culture.

9. The method of claim 7 or 8, wherein the culture pH of about 6.5 to about 7.0 is maintained from culture day 3 until harvest of the cell culture.

10. The method of claim 8 or 9, wherein the cumulative culture time that pH is allowed to drop below a fixed pH setpoint and the cumulative magnitude of the drop, after the pH is lowered on day 3 (integrated pH2 difference), is less, relative to a cell culture with an integrated pH2 difference that is more.

11. The method of claim 10, wherein the fixed pH setpoint is pH 6.91.

12. The method of claim 10 or 11, wherein lower integrated pH2 difference results in higher integrated viable cell density (IVCD) and higher titer at harvest.

13. The method of any one of claims 10 to 12, wherein lower integrated pH2 difference further results in lower percent fucosylation.

14. The method of any one of claims 1 to 13, wherein the rat hybridoma cells expressing the antibody protein are cultured in a chemically defined and animal-derived component free (ADCF) culture medium.

15. The method of any one of claims 1 to 14, wherein harvest titer of the antibody protein is increased and/or fucosylation of the antibody protein is decreased when the culture pH is 6.6 to 6.96 relative to a cell culture under the same culture conditions except that the culture pH is 6.60 to 6.80.

16. The method of any one of claims 1 to 15, further comprising controlling culture pCO₂ levels to less than about 300 mmHg.

17. The method of claim 16, wherein pCO₂levels less than about 300 mmHg are facilitated by supplementing the cell culture with additional buffers, increasing the air sparge rate, increasing the dissolved oxygen (DO) setpoint, and/or decreasing the agitation rate.

18. The method of any one of claims 1 to 17, further comprising an initial temperature set point of about 37° C., wherein said initial temperature set point is set on culture day 0 to culture day 1.

19. The method of claim 18, further comprising a second temperature set point of about 35° C., wherein said second temperature set point is set at the end of culture day 1 to culture day 3.

20. The method of claim 19, wherein the end of culture day one is 17 to 33 hours after the start of the cell culture.

21. The method of any one of claims 18 to 20, further comprising a third temperature set point of about 32° C. to about 33° C., wherein said third temperature set point is set on culture day 3 and is maintained though harvest.

22. The method of claim 21, wherein the third temperature set point is 32.5° C.

23. The method of any one of claims 1 to 22, wherein the cell culture comprises the following culture conditions:
i) an initial temperature set point of about 37° C., wherein said initial temperature set point is set on culture day 0 to culture day 1; a second temperature set point of about 35° C., wherein said second temperature set point is set at the end of culture day 1 to culture day 3; and a third temperature set point of about 32.5° C., wherein said third temperature set point is set on culture day 3 and is maintained though harvest;
ii) a culture pH between about 6.5 to about 7.55; and
iii) a culture pCO₂ less than about 300 mmHg.

24. The method of any one of claims 1 to 23, wherein yield of the antibody protein is increased by at least about 30%, at least about 40%, at least about 50%, at least about 60%, at least about 70%, at least about 80%, at least about 90%, at least about 100%, at least about 110%, at least about 120%, at least about 130%, at least about 140%, or at least about 150%, relative to an antibody protein produced by a culturing process that does not employ the culture conditions recited in claim 23.

25. The method of any one of claims 1 to 24, further comprising harvesting the antibody protein produced by the rat hybridoma cell.

26. The method of claim 25, further comprising purifying the antibody protein by affinity chromatography and/or ion exchange chromatography, optionally wherein the affinity chromatography comprises protein A purification.

27. The method of claim 26, wherein the purified antibody protein produced by the rat hybridoma cell is formulated into a pharmaceutically acceptable formulation.

28. The method of claim 26 or 27, wherein quality of the purified antibody protein is measured by SEC-HPLC, imaged capillary electrophoresis (ICIEF), and/or N-linked glycan analysis.

29. The method of any one of claims 1 to 28, wherein the antibody protein is a monoclonal antibody.

30. The method of claim 29, wherein the monoclonal antibody is an anti-CD20 antibody.

31. The method of claim 29 or 30, wherein the monoclonal antibody is subject to CD20, FcγRIIIa-158V, and/or C1q binding assays.

32. The method of claim 31, wherein the monoclonal antibody has a relative potency of 82% to 138% in a cell-based CD20 binding activity bioassay compared to that of a commercial reference standard.

33. The method of claim 32, wherein CD20 binding is determined by the binding of an anti-CD20 antibody to the CD20 expressing human mantle cell lymphoma cell line, Jeko-1.

34. The method of any one of claims 31-33, wherein the percentage of FcγRIIIa-158V binding is about 82% to about 130% with respect to a commercial reference standard for the binding assay.

35. The method of claim 34, wherein the percentage of FcγRIIIa-158V binding is determined by surface plasmon resonance (SPR).

36. The method of any one of claims 31-35, wherein the monoclonal antibody has a relative potency of 86% to 117% in a C1q binding assay as measured by ELISA compared to a commercial reference standard.

37. The method of claim 36, wherein the monoclonal antibody has a relative potency of 88% to 113% in a C1q binding assay as measured by ELISA compared to a commercial reference standard.

38. The method of any one of claims 29-37, wherein the monoclonal antibody has a relative potency of 74% to 127% in a cell-based complement dependent cytotoxicity (CDC) assay compared to that of a commercial reference standard.

39. The method of any one of claims 29-38, wherein the monoclonal antibody produced has higher percent antibody-dependent cellular cytotoxicity (ADCC) activity relative to a monoclonal antibody produced by a culturing process that does not employ the culture conditions recited in claim 23.

40. The method of claim 38, wherein the monoclonal antibody has a relative potency of 90% to 163% in a cell-based ADCC assay compared to a commercial reference standard.

41. The method of claim 40, wherein the monoclonal antibody has a relative potency of about 117% in a cell-based ADCC assay compared to a commercial reference standard.

42. The method of any one of claims 30-41, wherein the monoclonal antibody comprises:
a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and
a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

43. The method of claim 42, wherein the monoclonal antibody comprises a heavy chain having at least 95% identity to an amino acid sequence set forth in SEQ ID NO:7 and a light chain having at least 95% identity to an amino acid sequence set forth in SEQ ID NO:8.

44. The method of claim 42 or 43, wherein the monoclonal antibody comprises a heavy chain having an amino acid sequence set forth in SEQ ID NO:7 and a light chain having an amino acid sequence set forth in SEQ ID NO:8.

45. The method of claim 42 or 43, wherein the monoclonal antibody comprises a heavy chain having an amino acid sequence set forth in SEQ ID NO:7 and a light chain having an amino acid sequence set forth in SEQ ID NO:9.

46. The method of any one of claims 42-45, wherein the monoclonal antibody comprises a deletion of up to 5 N-terminal residues.

47. The method of any one of claims 42-45, wherein the monoclonal antibody comprises a deletion of up to 10 N-terminal sequences.

48. The method of any one of claims 1-47, wherein the cell culture is conducted in a bioreactor.

49. The method of claim 48, wherein the bioreactor is a commercial-scale bioreactor.

50. The method of claim 49, wherein the commercial-scale bioreactor is a 10,000 L, 15,000 L, 20,000 L, or 25,000 L bioreactor.

51. The method of claim 50, wherein the commercial-scale bioreactor is a 15,000 L bioreactor.

52. The method of any one of claims 1-51, wherein the rat hybridoma cell is a YB2/0 rat hybridoma cell.

53. A method of making an antibody protein in a culture of rat hybridoma cells at a commercial-scale comprising the steps of:
a) preparing and thawing a working rat hybridoma cell bank of the antibody protein of interest;
b) expanding a culture of the rat hybridoma cells from the cell bank by size and volume though a series of shake flasks (125 mL, 500 mL, 3 L, 3×3 L shake flasks, and 50 L cellbag) with a targeted seeding density of at least 0.30×10⁶ viable cells/mL;
c) processing the cell culture through a series of seed bioreactors (120 L, 600 L, and 3,000 L) to further increase the volume and cell culture mass;
d) inoculating the cell culture from the 3,000 L seed bioreactor into a commercial-scale production bioreactor;
e) harvesting the cell culture supernatant from the commercial scale production bioreactor;
f) clarifying the recovered cells by continuous centrifugation followed by depth filtration;
g) purifying the antibody protein by Protein A capture column chromatography; and
h) inactivating viral agents by solvent detergent viral inactivation (SDVI).

54. The method of claim 53, wherein the commercial-scale production bioreactor is operated in fed-batch mode.

55. The method of claim 53 or 54, wherein a drilled hole (10) gas sparger with 4.0 mm orifice diameter is used in the commercial-scale production bioreactor.

56. The method of any one of claims 53-55, further comprising purification by cation exchange chromatography (CEX) and anion exchange chromatography (AEX).

57. The method of any one of claims 53-56, further comprising viral filtration (VF) to remove potential viruses.

58. The method of any one of claims 53-57, further comprising ultrafiltration/diafiltration (UFDF).

59. The method of any one of claims 53-58, further comprising preparing a bulk drug substance formulation comprising the antibody protein, comprising adding polysorbate 80 in formulation buffer to prepare a bulk drug substance formulation.

60. The method of claim 59, further comprising subjecting the bulk drug substance formulation to 0.2 μm filtration.

61. The method of claim 59 or 60, further comprising filling a 6 L bag to a target fill volume of 5.50 L of the bulk drug substance formulation and storing the bulk drug substance formulation at ≤−35° C.

62. The method of any one of claims 59-61, wherein the antibody protein in the bulk drug substance formulation is formulated into a pharmaceutically acceptable formulation.

63. The method of any one of claims 59-62, further comprising testing unprocessed bulk harvest from the commercial-scale production bioreactor for microbial and viral adventitious agents.

64. The method of claim 63, further comprising removing and/or inactivating the microbial and viral adventitious agents from the commercial-scale production bioreactor.

65. The method of any one of claims 53-64, wherein the rat hybridoma cells are YB2/0 cells.

66. The method of any one of claims 53-65, wherein the antibody protein is a monoclonal antibody, optionally wherein the monoclonal antibody is an anti-CD20 antibody.

67. The method of claim 66, wherein the monoclonal antibody comprises:
a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and
b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

68. The method of claim 67, wherein the monoclonal antibody comprises a heavy chain having at least 95% identity to an amino acid sequence set forth in SEQ ID NO:7; and a light chain having at least 95% identity to an amino acid sequence set forth in SEQ ID NO: 8.

69. The method of claim 67 or 68, wherein the monoclonal antibody comprises a heavy chain having an amino acid sequence set forth in SEQ ID NO:7; and a light chain having an amino acid sequence set forth in SEQ ID NO:8.

70. The method of claim 67 or 68, wherein the monoclonal antibody comprises a heavy chain having an amino acid sequence set forth in SEQ ID NO:7; and a light chain having an amino acid sequence set forth in SEQ ID NO:9.

71. The method of any one of claims 1-70, wherein the antibody protein comprises an N-glycan profile comprising one or both of the following:
i) about 10 to 20% galactosylated glycans; and/or
ii) about 20 to 40% fucosylated glycans.

72. The method of claim 71, wherein the N-glycan profile comprises about 10 to 20% galactosylated glycans and about 23% to 36% fucosylated glycans.

73. The method of claim 71 or 72, wherein the N-glycan profile comprises about 23% to about 36% fucosylated glycans.

74. The method of any one of claims 71-73, wherein the N-glycan profile comprises about 16% to about 18% galactosylated glycans, optionally about 17% galactosylated glycans.

75. The method of any one of claims 1-74, wherein the antibody protein comprises an N-glycan profile comprising at least about 10% bisecting N-glycans.

76. The method of claim 75, wherein the N-glycan profile comprises about 12% to about 30% bisecting N-glycans.

77. The method of claim 76, wherein the N-glycan profile comprises about 18% bisecting N-glycans.

78. The method of any one of claims 1-77, wherein the antibody protein comprises an N-glycan profile comprising less than 5% sialylated glycans.

79. The method of claim 78, wherein the N-glycan profile comprises less than 4%, 3%, 2.5%, 2%, 1%, or 0.5% sialylated glycans.

80. The method of claim 78 or 79, wherein the N-glycan profile comprises no detectable amount of sialylated glycan.

81. The method of any one of claims 1-80, wherein the antibody protein comprises an N-glycan profile comprising 0.1% to 1.5% Man5 N-glycan.

82. The method of claim 81, wherein the N-glycan profile comprises 0.4% to 0.7% Man5 N-glycan.

83. The method of claim 82, wherein the N-glycan profile comprises about 0.6% Man5 N-glycan.

84. The method of any one of claims 81-83, wherein Man5 N-glycan is the only high mannose species in the N-glycan profile.

85. The method of any one of claims 1-84, wherein the antibody protein is produced at a commercial-scale of about 10,000 L to about 25,000 L.

86. The method of claim 85, wherein the commercial-scale is 15,000 L.

87. The method of any one of claims 1-86, wherein the method yields an antibody protein harvest titer of about 0.5 g/L to about 1.5 g/L.

88. The method of claim 87, wherein the harvest titer is about 1.0 g/L to about 1.5 g/L.

89. An antibody protein made according to the method of any one of claims 1-88.

90. The antibody protein of claim 89, wherein the antibody protein is a monoclonal antibody, optionally wherein the monoclonal antibody is an anti-CD20 antibody.

91. A rat hybridoma master cell bank (MCB) composition comprising an antibody protein having at least two of the following parameters:
i) peak viable cell density of about 11 to about 13×10⁶ cells/mL;
ii) harvest titer of about 650 to about 720 mg/L;
iii) percent fucosylation of about 30% to about 38%;
iv) about 97% to about 99% monomers as detected by size exclusion chromatography (SEC);
v) about 1.5% to about 2% dimers as detected by SEC;
vi) undetectable to about 3% level of aggregates as detected by SEC;
vii) undetectable to about 1% level of fragments as detected by SEC;
viii) about 25% to about 30% acidic isoforms as detected by imaged capillary isoelectric focusing (iCIEF);
ix) about 38% to about 49% main isoforms as detected by iCIEF; and/or
x) about 20% to about 36% basic isoforms as detected by iCIEF.

92. A rat hybridoma working cell bank (WCB) composition comprising an antibody protein having at least two of the following parameters:
i) peak viable cell density that is about 11 to about 28×10⁶ cells/mL;
ii) harvest titer that is about 420 to about 1280 mg/L;
iii) percent fucosylation of about 18% to about 40%;
iv) about 97% to about 99% monomers as detected by size exclusion chromatography (SEC);
v) about 1% to about 2% dimers as detected by SEC;
vi) undetectable to about 2% level of aggregates as detected by SEC;
vii) undetectable to about 1% level of fragments as detected by SEC;
viii) about 19% to about 31% acidic isoforms as detected by imaged capillary isoelectric focusing (iCIEF);
ix) about 34% to about 62% main isoforms as detected by iCIEF; and/or
x) about 14% to about 38% basic isoforms as detected by iCIEF.

93. The MCB composition of claim 91 or the WCB composition of claim 92, wherein the rat hybridoma cells in the cell bank are YB2/0 cells.

94. The MCB or WCB composition of claim 93, wherein the antibody protein is a monoclonal antibody, optionally wherein the monoclonal antibody is an anti-CD20 antibody.

95. The MCB or WCB composition of claim 94, wherein the anti-CD20 antibody comprises:
a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and
b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

96. The MCB or WCB composition of claim 95, wherein the anti-CD20 antibody comprises a heavy chain having at least 95% identity to an amino acid sequence set forth in SEQ ID NO:7; and a light chain having at least 95% identity to an amino acid sequence set forth in SEQ ID NO: 8.

97. The MCB or WCB composition of claim 95 or 96, wherein the anti-CD20 antibody comprises a heavy chain having an amino acid sequence set forth in SEQ ID NO:7; and a light chain having an amino acid sequence set forth in SEQ ID NO:8.

98. The MCB or WCB composition of claim 95 or 96, wherein the anti-CD20 antibody comprises a heavy chain having an amino acid sequence set forth in SEQ ID NO:7; and a light chain having an amino acid sequence set forth in SEQ ID NO:9.

99. A method of making an antibody protein by using the MCB or WCB composition of any one of claims 91-98.

100. The method of claim 99, wherein the antibody protein is a monoclonal antibody, optionally wherein the monoclonal antibody is an anti-CD20 antibody.

101. The method of claim 100, wherein the anti-CD20 antibody comprises:
a) a heavy chain CDR1 having an amino acid sequence set forth in SEQ ID NO: 1; a heavy chain CDR2 having an amino acid sequence set forth in SEQ ID NO: 2; and a heavy chain CDR3 having an amino acid sequence set forth in SEQ ID NO: 3; and
b) a light chain CDR1 having an amino acid sequence set forth in SEQ ID NO: 4; a light chain CDR2 having an amino acid sequence set forth in SEQ ID NO: 5; and a light chain CDR3 having an amino acid sequence set forth in SEQ ID NO: 6.

102. The method of claim 101, wherein the anti-CD20 antibody comprises a heavy chain having at least 95% identity to an amino acid sequence set forth in SEQ ID NO:7; and a light chain having at least 95% identity to an amino acid sequence set forth in SEQ ID NO: 8.

103. The method of claim 101 or 102, wherein the anti-CD20 antibody comprises a heavy chain having an amino acid sequence set forth in SEQ ID NO:7 and a light chain having an amino acid sequence set forth in SEQ ID NO:8.

104. The method of claim 101 or 102, wherein the anti-CD20 antibody comprises a heavy chain having an amino acid sequence set forth in SEQ ID NO:7; and a light chain having an amino acid sequence set forth in SEQ ID NO:9.


SEQUENCE LISTING (17 sequences)

SEQ ID NO: 1 - protein, 7 aa, synthetic construct
GYTFTSY

SEQ ID NO: 2 - protein, 6 aa, synthetic construct
YPGNGD

SEQ ID NO: 3 - protein, 9 aa, synthetic construct
YDYNYAMDY

SEQ ID NO: 4 - protein, 10 aa, synthetic construct
RASSSVSYMH

SEQ ID NO: 5 - protein, 7 aa, synthetic construct
ATSNLAS

SEQ ID NO: 6 - protein, 9 aa, synthetic construct
QQWTFNPPT

SEQ ID NO: 7 - protein, 448 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 213 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 118 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 11 - protein, 330 aa, synthetic construct [sequence omitted]

SEQ ID NO: 12 - protein, 107 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIKR

SEQ ID NO: 13 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1344 bp, synthetic construct
  source 1..1344: mol_type = other DNA
caggcttatctacagcagtctggggctgagctggtgaggcctggggcctcagtgaagatgtcctgcaaggcttctggctacacatttaccagttacaatatgcactgggtaaagcagacacctagacagggcctggaatggattggaggtatttatccaggaaatggtgatacttcctacaatcagaagttcaagggcaaggccacactgactgtaggcaaatcctccagcacagcctacatgcagctcagcagcctgacatctgaagactctgcggtctatttctgtgcaagatatgactacaactatgctatggactactggggtcaaggaacctcagtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 15 - DNA, 639 bp, synthetic construct
  source 1..639: mol_type = other DNA
caaattgttctctcccagtctccagcaatcctgtctgcatctccaggggagaaggtcacaatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttattctttcacaatcagcagagtggaggctgaagatgctgccacttattactgccagcagtggacttttaacccacccacgttcggaggggggaccaggctggaaataaaacggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - protein, 5 aa, Homo sapiens
IRAHT

SEQ ID NO: 17 - protein, 4 aa, Homo sapiens
EPAN